(19) United States
(12) Patent Application Publication
(10) Pub. No.: US 2022/0241369 A1
(43) Pub. Date: Aug. 4, 2022

(54) COMPOSITIONS AND METHODS OF MODULATING INFLAMMATION

(71) Applicant: La Jolla Institute for Allergy and Immunology, La Jolla, CA (US)

(72) Inventors: Toshiaki KAWAKAMI, Del Mar, CA (US); Yuko KAWAKAMI, Del Mar, CA (US)

(73) Assignee: La Jolla Institute for Allergy and Immunology, La Jolla, CA (US)

(21) Appl. No.: 17/292,639

(22) PCT Filed: Nov. 11, 2019
(86) PCT No.: PCT/US2019/060787

§ 371 Date: May 10, 2021

Related U.S. Application Data
(60) Provisional application No. 62/760,021, filed on Nov. 12, 2018.

(51) Int. Cl. A61K 38/16; A61P 37/08; A61P 29/00

(52) U.S. Cl. CPC A61K 38/16; A61P 29/00; A61P 37/08

(57) ABSTRACT

Methods are disclosed for identifying whether a subject is likely to respond to a therapy for treatment of an allergy (e.g., a food allergy or an allergic reaction), hypersensitivity, asthma, inflammatory response or inflammation by detecting the level of an HRF monomer, an HRF dimer, an HRF multimer, or an HRF-reactive Ig molecule in a sample isolated from the subject. Also provided are methods for diagnosing or determining the severity of a condition selected from an allergy (e.g., a food allergy or an allergic reaction), hypersensitivity, asthma, inflammatory response or inflammation/or severity of allergic reaction, or inflammation in a subject. Treatments for allergic conditions are further provided herein, as well as kits for the diagnosis, monitoring and treatment of the conditions.

### CROSS REFERENCE TO RELATED APPLICATIONS

This application is a U.S. National Phase Application under 35 U.S.C. § 371 of International Application No. PCT/US2019/060787, filed on Nov. 11, 2019, which claims the benefit under 35 U.S.C. § 119(e) of U.S. Provisional Application Ser. No. 62/760,021, filed Nov. 12, 2018, the contents of which are incorporated by reference herein in their entirety including all figures and tables.

### SEQUENCE LISTING

The instant application contains a Sequence Listing which has been submitted electronically in ASCII format and is hereby incorporated by reference in its entirety. Said ASCII copy, created on Jan. 24, 2022, is named “116639-0411_SL.txt” and is 18 kilobytes in size.

### BACKGROUND

The prevalence of food allergy has been dramatically increasing for the last few decades (1, 2). Six to eight percent of children under the age of three have food allergies and nearly four percent of adults have them. Symptoms of food allergy range from itching, hives, and diarrhea to life-threatening anaphylaxis. Currently, there is no cure for this disease, although allergen-specific immunotherapy can successfully treat some patients (3-6). A need exists in the art for safe and effective treatment of all allergic reactions, include food allergies, as well as diagnosing and monitoring treatment. This disclosure satisfies this need and provides related advantages as well.

### SUMMARY OF THE DISCLOSURE

Provided herein is a method for identifying a subject suffering from an allergy likely to respond to an allergen immunotherapy, comprising, or alternatively consisting essentially of, or yet further consisting of, contacting a sample isolated from the subject with an agent that detects an HRF-reactive Ig molecule, and detecting the amount of HRF-reactive Ig molecule in the sample. In one aspect, this disclosure provides a method for monitoring allergen immunotherapy in a subject, the method comprising, or alternatively consisting essentially of, or yet further consisting of, contacting a sample isolated from the subject with an agent that detects an HRF-reactive Ig molecule, and detecting the amount of HRF-reactive Ig molecule in the sample. In another aspect, it also discloses a method to monitor therapy for treatment of allergic reaction, hypersensitivity, asthma, inflammatory response or inflammation in a subject, comprising detecting the level of HRF monomer, HRF dimer, HRF multimer, or HRF-reactive Ig molecule in a sample isolated from the subject, the method comprising, or alternatively consisting essentially of, or yet further consisting of, contacting the sample with an agent that detects HRF monomer, HRF dimer, HRF multimer, or HRF-reactive Ig molecule. In a further aspect, provided herein is a method of identifying a subject that will or is likely to respond to therapy for treatment of allergic reaction, hypersensitivity, asthma, inflammatory response or inflammation, comprising, or alternatively consisting essentially of, or yet further consisting of, detecting the level of HRF monomer, HRF dimer, HRF multimer, or HRF-reactive Ig molecule in a sample isolated from the subject, the method comprising, or alternatively consisting essentially of, or yet further consisting of, contacting the sample with an agent that detects HRF monomer, HRF dimer, HRF multimer, or HRF-reactive Ig molecule. In yet another aspect, this disclosure provides a method for diagnosing or determining the severity of a condition selected from the group of: allergic reaction, hypersensitivity, asthma, inflammatory response or inflammation/or severity of allergic reaction, or inflammation in a subject, comprising, or alternatively consisting essentially of, or yet further consisting of, detecting the level of HRF monomer, HRF dimer, HRF multimer, or HRF-reactive Ig molecule in a sample isolated from the subject, the method comprising contacting the sample with an agent that detects HRF monomer, HRF dimer, HRF multimer, or HRF-reactive Ig molecule. In a further aspect, it discloses a method for the treatment of a condition from the group of: allergic reaction, hypersensitivity, asthma, inflammatory response or inflammation in a subject in need thereof, comprising, or alternatively consisting essentially of, or yet further consisting of, administering an effective amount of an agent that interferes with the formation of HRF-Ig complexes to the subject, thereby treating the condition.

In additional aspects, provided herein is a method for identifying or assessing whether a subject is likely to respond to a therapy for treatment of an allergy, hypersensitivity, asthma, inflammatory response or inflammation, comprising: contacting a sample isolated from the subject with an agent that detects an HRF monomer, an HRF dimer, an HRF multimer, or an HRF-reactive immunoglobulin (Ig) molecule; and detecting an amount of the HRF monomer, the HRF dimer, the HRF multimer, or the HRF-reactive Ig molecule in the sample.

In another aspect, disclosed herein is a method for diagnosing or determining the severity of a condition selected from an allergy, hypersensitivity, asthma, inflammatory response or inflammation in a subject, comprising contacting a sample isolated from the subject with an agent that detects an HRF monomer, an HRF dimer, an HRF multimer, or an HRF-reactive immunoglobulin (Ig) molecule; and detecting an amount of the HRF monomer, the HRF dimer, the HRF multimer, or the HRF-reactive Ig molecule in the sample. In a further aspect, disclosed herein is a method of monitoring a therapy for treatment of an allergy, hypersensitivity, asthma, inflammatory response or inflammation in a subject in need thereof, comprising contacting a sample isolated from the subject with an agent that detects an HRF monomer, an HRF dimer, an HRF multimer, or an HRF-reactive immunoglobulin (Ig) molecule to determine the presence and amount of the HRF monomer, the HRF dimer, the HRF multimer, or the HRF-reactive Ig molecule in the sample.

### BRIEF DESCRIPTION OF THE FIGURES

FIGS. 1A-1I show GST-N19 inhibits the development of diarrhea and intestinal mastocytosis in murine food allergy. Mice were i.p. sensitized with OVA (50 mg/mouse) plus alum on days 0 and 14. From day 28, mice were i.g. challenged with OVA (50 mg) three times a week. Sensitized mice were either non-pretreated (−) or i.g. pretreated with 100 μg of GST or GST-N19 (N19). (FIG. 1A) Procedure scheme. (FIG. 1B) The development of diarrhea was monitored for 90 min after each OVA challenge. (n=5 for non-sensitized group, n=3 for inhibitor non-pretreated group, n=4 for GST group, n=6 for N19 group, data are representative of at least 3 independent experiments) Log-rank test: p=0.028 (GST vs. GST-N19), p=0.036 (control vs. GST-N19). Non-sensitized mice did not exhibit diarrhea after OVA gavages (not shown). (FIG. 1C-FIG. 1E) Total IgE, IgG1, HRF, and HRF-reactive IgE and IgG in sera were measured by ELISA. (FIG. 1F) Serum mMCP-1 was also measured by ELISA. (FIG. 1G) Sections of jejunum were stained with chloroacetate esterase to identify mast cells. Sections indicated by rectangles are enlarged in lower rows. Bar=100 μm in large pictures and 50 μm in insets. (FIG. 1H) Flow cytometry was performed to enumerate MMC9s (Lin⁻c-Kit⁺ST2⁺IL-17RB⁻Integrin β7^{lo}) in jejunum. (n=14 for non-sensitized group, n=15 for inhibitor non-pretreated group, n=9 for GST group, n=9 for GST-N19 group, pooled data of 3 independent experiments) (FIG. 1I) Cytokine mRNAs in jejunum were quantified by qRT-PCR. *, **: p<0.05, p<0.01 by ANOVA with Tukey's multiple comparison.

FIGS. 2A-2C show mucosal mast cells can be activated ex vivo by antigen. Cells were released with EDTA from small intestines of non-sensitized/OVA challenged (Cont) or OVA-sensitized/OVA-challenged (OVA) mice, and mononuclear cells were selected. The cells were incubated for 15 min at 37° C. with OVA or anti-IgE antibody, and activation of Kit⁺ mast cells was measured by flow cytometry (FIG. 2A, FIG. 2B). **, ***: p<0.01, p<0.001 by Student's t-test. (n=2; a representative of 2 independent experiments) (FIG. 2C) Mucosal cells derived from OVA-sensitized/OVA-challenged mice were stimulated with PBS (n=4), OVA (10 ng/ml, n=4) or HRF dimer (100 μg/ml, n=2) in the presence or absence of HRF-2CA (300 μg/ml).

FIGS. 3A-3E show HRF dimer is increased in allergic mice and GST-N19 preferentially targets mast cells in jejunum. HRF multimers in the jejunum of non-sensitized/OVA challenged (Cont, n=6) or OVA-sensitized/OVA-challenged (OVA, n=5) mice were quantified by a newly developed ELISA (FIG. 3A, FIG. 3C). HRF was also measured by ELISA (FIG. 3B). HRF dimer and monomer were quantified by western blotting in control (Cont) and food allergic mice treated without (OVA) or with GST (GST) or GST-N19 (N19) (n=5 for each group) (FIG. 3D). Allergic diarrhea was induced by 6 challenges. On the day of 7th challenge, the mice were gavaged with 0.1 mg of GST or GST-N19 (FIG. 3E). One hour later, mice were sacrificed without OVA challenge. Jejunum tissues were stained for GST, IgE, and mMCP-1 before confocal microscopy (n=5 for GST and n=6 for GST-N19). Arrows indicate cells positive for GST or GST-N19 as well as IgE and mMCP-1. The right graph shows quantitation. *, **, ***: p<0.05, p<0.01, p<0.001 by Student's t-test. Bars=50 μm.

FIGS. 4A-4C show HRF is secreted by various cells. CMT-93 murine intestinal epithelial cells (FIG. 4A) and NIH/3T3 murine fibroblasts (FIG. 4B) and Raw264.7 murine macrophages (FIG. 4C) were stimulated overnight with low (20 ng/ml) and high (100 ng/mL) concentrations of various cytokines. HRF secreted into culture supernatants was detected by western blotting. Med and US denote medium only and supernatant from unstimulated cells, respectively. −DTT and +DTT indicate SDS gels were run under non-reducing and reducing conditions, respectively. Gel portions of containing the 23 kDa HRF monomer are shown except that a larger portion of the non-reducing gel is shown in panel C. Arrow indicates the position of HRF dimer, which disappeared under reducing conditions.

FIGS. 5A-5F show therapeutic treatment with HRF inhibitor ameliorates the severity of food allergy. Mice were sensitized and i.g. challenged with OVA. By the seventh OVA challenge, all the mice suffered diarrhea. These mice were pretreated i.v. with 30 μg of HRF-2CA (2CA) or PBS (OVA) before challenged with OVA two more times. (FIG. 5A) Procedure scheme. (FIG. 5B) Diarrhea severity (n=11 for OVA, n=13 for HRF-2CA and n=4 for non-sensitized control (not shown); pooled data of two independent experiments). ***: p<0.001 by two-way ANOVA. (FIG. 5C) Total and OVA-specific IgE and IgG1 concentration in sera were measured by ELISA. (FIG. 5D) mMCP-1 in sera was measured by ELISA. (FIG. 5E) Sections of jejunum were stained with chloroacetate esterase. (FIG. 5C—FIG. 5E) *, **, ***: p<0.05, p<0.01, p<0.001 by Student's t-test. (FIG. 5F) Similar i.v. therapeutic treatment of allergic mice was conducted with 30 μg of synthetic N19 peptide (n=12 for OVA and n=12 for pepN19; pooled data of two independent experiments). **: p<0.001 by two-way ANOVA.

FIGS. 6A-6J show reduced HRF-reactive IgE levels correlate with effective OIT outcomes in food allergy. (FIG. 6A) Scheme of a rush OIT. (FIG. 6B—FIG. 6G) HRF-reactive IgE (FIG. 6B—FIG. 6D) and IgG (FIG. 6E-FIG. 6G) levels were measured by ELISA before, one week after, and 12 months after OIT initiation in normal subjects (NS) and food allergy patients (FA). (FIG. 6B, FIG. 6E) Data represent all the patients (n=9 for NS and n=42 for FA; samples from one patient was not available due to sample shortage). (FIG. 6C, FIG. 6F) Data represent patients who underwent the full OIT regimen (n=37). (FIG. 6D, FIG. 6G) Data represent patients showing considerable and severe reductions in threshold (n=7). Analyzed by Student's t-test (FIG. 6B, FIG. 6E) and Paired t-test (FIG. 6C, FIG. 6D, FIG. 6F, FIG. 6G). (FIG. 6H) HRF-reactive IgE increase indices (Supplemental Procedure) were compared among the four cohorts by ANOVA with Holm-Sidak's multiple comparison (n=17 for stable desensitization, n=13, 4 and 3 for patients with reduced threshold to 1000, 300 and 100 mg of egg-white powder, respectively). (FIG. 6I) Distribution of HRF-reactive IgE and egg white-specific IgE levels before OIT in egg allergy patients (n=37). (FIG. 6J) Comparison of receiver operating characteristic (ROC) curves showing the performances of initial HRF-reactive IgE (solid line), initial egg-white specific IgE (broken line), and a combination of both factors (bold line) in predicting prolonged desensitization at 12 months after OIT. AUC, area under the curve.

FIGS. 7A-7E show HRF-reactive IgE levels are low in desensitized mice. (FIG. 7A) Mice were unsensitized (Cont) or i.p. sensitized as described in the FIG. 1 legend, and i.g. challenged with OVA with non-pretreated (OVA) or i.g. pretreated with HRF-2CA (2CA). HRF-reactive IgE and IgG and OVA-specific IgE and IgG after sensitization (before OVA gavages) and after OVA gavages were measured (n=4 for non-sensitized group, n=14 for OVA group and n=14 for 2CA group; pooled data of two independent experiments). **, p<0.01; ***, p<0.001; ****, p<0.0001 by paired t test. (FIG. 7B, FIG. 7C) Five daily oral administrations of 1 mg OVA from day −11 to day −7 were followed by sensitization with OVA and then by OVA challenges in mice. Without oral pretreatment, which prevented diarrhea, all OVA-sensitized mice developed allergic diarrhea. At the end of experiment, HRF-reactive IgE and total IgE were quantified (n=5 for non-sensitized group, n=10 for OVA group, and n=9 for OIT/OVA group; pooled data of two independent experiments). (FIG. 7D, FIG. 7E) Mice were intradermally sensitized with OVA and followed by oral OVA challenges. All mice showed diarrhea. Then some mice received hourly increasing amounts of OVA for 3 consecutive days (OIT). Levels of HRF-reactive IgE and IgG as well as OVA-specific IgE and IgG were quantified by ELISA before and after OIT (n=4 for no OIT group and n=5 for OIT group). AU, arbitrary unit. Statistics by paired t test.

FIGS. 8A-8F show HRF-2CA inhibits the development of diarrhea and intestinal mucosal mastocytosis. Mice were immunized and i.g. challenged with OVA as described in the FIG. 1 legend. Mice were pretreated with 100 μg of HRF-2CA (2CA) or PBS (OVA) before OVA gavages. (FIG. 8A) Procedure scheme. (FIG. 8B) The occurrence of diarrhea. Log-rank test: p=0.0499 (PBS n=10 vs. HRF-2CA n=10; pooled data of two independent experiments). (FIG. 8C) Total IgE and IgG1, and (FIG. 8D) mMCP-1 concentrations in sera were measured by ELISA. (FIG. 8E, FIG. 8F) Sections of jejunum were stained with chloroacetate esterase to quantify mucosal (FIG. 8E) and submucosal (FIG. 8F) mast cells. *, **, ***: p<0.05, p<0.01, p<0.001 by ANOVA with Tukey's multiple comparison.

FIGS. 9A-9D show HRF inhibitors do not affect the sensitization phase of murine food allergy. Mice were i.g. pretreated with 100 μg of GST, GST-N19, HRF-2CA or PBS (OVA) one day before i.p. immunization with OVA plus alum on days 0 and 14. From day 28, mice were i.g. challenged with OVA three times a week. (FIG. 9A) Procedure scheme. (FIG. 9B) The development of diarrhea was monitored after OVA challenge. (n=5 for OVA, n=4 for GST, n=5 for N19, n=5 for 2CA, and n=3 each for unsensitized pretreated groups (not shown)). (FIG. 9C, FIG. 9D) Total IgE and IgG1 were measured by ELISA.

FIGS. 10A-10C show HRF-2CA suppresses allergic diarrhea via FcεRI. WT and mutant mice were subjected to food allergy experiments as described in the FIG. 1 legend. FceRIa^{-/-} (n=4-5 each for group with sensitization and n=3 each for unsensitized group (Cont; no diarrhea was observed))(FIG. 10A), FcgRIIB^{-/-} (n=9-10 each for groups with sensitization and n=4-5 each for unsensitized groups (Cont; no diarrhea was observed); pooled data of two independent experiments) (FIG. 10B), and Enpp3^{-/-} (n=9-10 each for groups with sensitization and n=7 each for unsensitized groups (Cont; no diarrhea was observed); pooled data of two independent experiments) (FIG. 10C) mice along with WT mice were used. *, ***: p<0.05, p<0.001 by Student's t-test.

FIGS. 11A-11C show HRF dimer, but not monomer can activate mast cells. (FIG. 11A) Purified dimeric and monomeric HRF expressed in E. coli. Recombinant HRF-Hi₅₆ was purified first by Histrap HP and then Sephacryl S-200 HR16/60. Two peaks were observed by Sephacryl size fractionation. The first (Fr1) and second (Fr3) peaks contained dimer and monomer, respectively, as shown by SDS-PAGE. Fr1 and Fr3 were used for mast cell stimulation. (FIG. 11B) Cells were released with 10 mM EDTA from small intestines of OVA-sensitized/OVA-challenged mice, and mononuclear cells were selected. The cells were incubated for 15 min at 37° C. with mHRF monomer or dimer (100 μg/ml), or PBS, and Kit⁺ mast cell activation (LAMP-1⁺) was measured by flow cytometry (n=3). (FIG. 11C) Control experiments indicate an excellent specificity of the assay for HRF multimers. HRF-2CA (2CA) was used as a monomer control (n=2 for each concentration). Note that total concentrations of the 1:9 mixture of dimer:monomer gave almost the same luminescence values as did ten-fold less concentrations of dimer.

FIGS. 12A-12E show localization of HRF in the jejunum. (FIG. 12A) Jejunum from diarrheal mice was stained with indicated antibodies preincubated with or without recombinant HRF or BSA. Bound antibody was detected by Alexa Fluor 647-conjugated anti-rabbit antibody. Fluorescence was observed by confocal laser microscopy. (FIG. 12B-FIG. 12E) Jejunum from diarrheal mice were co-stained with anti-HRF and anti-CD45 (FIG. 12B), anti-IgE (FIG. 12C), anti-Siglec F (FIG. 12D), or anti-CD63 (FIG. 12E) antibodies. Fluorescence signal was detected and separated from autofluorescence by Nuance Multispectral Imaging System (PerkinElmer).

FIGS. 13A-13C show HRF is secreted from various cells. (FIG. 13A) Various cells were incubated overnight except for the NIH/3T3 cells, which were cultured for 4 or 12 h after confluency was reached. Culture supernatants were treated with DTT or not before run on an SDS gel. Western blot analysis was done with anti-HRF mAb to detect HRF monomer except for panel B, where both HRF dimers and monomers are shown in a non-reducing gel. (FIG. 13B) Bone marrow-derived eosinophils (>95% pure) were kept unstimulated (US) or stimulated overnight with the indicated cytokines (20 or 100 ng/ml). (FIG. 13C) Splenic T and B cells were stimulated overnight by the indicated cytokines (ng/ml). rHRF, recombinant HRF; Med, medium alone. Arrow indicates the position of HRF dimer.

FIG. 14 show HRF amplifies intestinal allergic inflammation. Epithelial damage or inflammation in the gut promotes increased entry of food allergens and secretion of the epithelial-derived cytokines TSLP, IL-25 and IL-33. These cytokines induce a Th2-skewed immune response. TSLP can enhance OX40L expression in dendritic cells, which induce Th2 cell differentiation of naïve CD4⁺ T cells. IL-25 secreted by tuft cells may help the expansion of type 2 innate lymphoid cells (ILC2). Th2 cells along with ILC2 cells promote the Th2 cell-mediated immune response, which includes IgE class switch recombination in B cells, eosinophil accumulation, and mastocytosis. IL-9 promotes the expansion of IL-9-producing mucosal mast cells (MMC9) as an important component of food allergy-associated inflammation. In this study, HRF dimer/multimers secreted from several types of cell amplify intestinal inflammation by activating IgE-bound mast cells synergistically with antigen via the FcεRI. This is likely due to increased HRF secretion by several types of cell in response to Th2, proinflammatory and even epithelial-derived cytokines.

FIG. 15 illustrates that using the objective parameter, temperature drop, the efficacy of HRF inhibitors was clearly shown in this and other experiments of food allergy.

FIG. 16 shows hypothermia and immobility can be used as important indicators of food allergy as compared to diarrhea, which has been used as a major indicator of food allergy. Hypothermia and activity can be exploited as the major means of severity judgement. Thus, in one aspect, hypothermia and activity can be used as an indication of treatment.

FIG. 17 shows comparisons between moderate and severe asthmatics and normal controls. Higher blood levels of HRF-reactive IgE were found in asthmatics, particularly patients with severe asthma, compared to normal controls. By contrast, HRF-reactive IgG levels were lower in asthmatics. Interestingly, HRF-reactive IgE (high) asthmatic group had a tendency to release more tryptase and more PGD2 upon anti-IgE stimulation of BAL cells. By contrast HRF-reactive IgG (high) group showed the opposite tendency.

FIG. 18 shows the results of a study wherein pediatric patients with asthma were compared to hospitalized patients without asthma. Asthmatics had higher blood levels of HRF-reactive IgE. Some of the control patients had mostly infectious diseases.

FIG. 19 shows the presence of 3 high-molecular-weight HRF species, most likely HRF multimers, in nasal washes in almost all individuals. When patients with mild asthma were infected with rhinovirus (RV), these high-molecular weight HRFs were dramatically increased at day 4, at the peak of asthma exacerbation, and then their amounts were decreased at day 21. Interestingly, 10 out of 10 asthmatics with RV-induced exacerbation showed high levels of HRF-reactive IgE, which declined after resolution of the exacerbation. By contrast, patients who showed asthma exacerbation without RV infection did not show higher HRF-reactive IgE at the exacerbation or their decline at the resolution. Thus, without being bound by theory, RV-induced asthma exacerbation may involve HRF-reactive IgE-mediated mast cell activation.

FIG. 20 shows the results of an HRF secretion study. As lung epithelial cells are exposed to the environment, HRF secretion was studied using BEAS-2B human bronchial epithelial cells. A small amount of HRF was secreted constitutively, but house dust mite allergens induced the secretion of high-molecular-weight HRF species (HMW-HRFs). Various cytokines including epithelial derived cytokines as well as Th2 cytokines and proinflammatory cytokines further enhanced HDM-induced secretion of HMW-HRFs.

FIG. 21 shows the results of a mechanical stress study. Cultured cells were scraped and treated with adenosine and ATP, which should be abundantly present in inflammatory or injured tissues. These conditions induced secretion of HRF monomer, dimer and HMW-HRFs like stimulation with HDM plus cytokines.

FIG. 22 shows that similar to nasal washes, saliva and tears also have HMW-HRFs, which are reduced to HRF monomer by DTT. (Left panel) Nasal washes were analyzed by SDS-PAGE under nonreducing (−DTT) and reducing (+DTT) conditions, followed by western blotting for probing with anti-HRF mAb. (Right panel) Similar to nasal washes, saliva and tears also have high-molecular-weight HRF species (HMW-HRFs), which are reduced to HRF monomer by DTT. HMW-HRFs include the 150 kDa hexamer, 240 kDa and >250 kDa species of HRF. Arrowhead indicates HRF monomer.

FIG. 23 shows the results of a study wherein BEAS-2B human bronchial epithelial cells were incubated for the indicated periods of time. Culture supernatants and cell lysates were analyzed by SDS-PAGE under nonreducing (−DTT) and reducing (+DTT) conditions, followed by western blotting for HRF probing. Arrowhead indicates HRF monomer and double arrowhead indicates HRF dimer. Recombinant HRF (rHRF) composed mostly of HRF dimer. Medium, culture medium alone.

FIG. 24 shows the results of a study wherein BEAS-2B human bronchial epithelial cells were incubated with allergens, cytokines or combinations of allergen and cytokine for 24 h. Culture supernatants were analyzed by SDS-PAGE under nonreducing (−DTT) and reducing (+DTT) conditions, followed by western blotting for HRF probing. HDM-induced HRF secretion was enhanced not only by Th2 cytokines (IL-4, IL-5, and IL-13), but also by epithelial derived cytokines (IL-25, IL-33, and TSLP) and proinflammatory cytokines (IL-1beta, IL-6 and TNF). med, medium alone; US, unstimulated; HDM, house dust mite; CR, cockroach; RW, ragweed; Af, Aspergillus fumigatus. Arrowhead, HRF monomer.

FIGS. 25A-25B show that HRF can be present as a monomer and homodimer. HRF dimer is linked via disulfide bonding at its C-terminal cysteine residue C172. The 3D structure of HRF dimer was determined and it was found that IgE-binding sites appear on the molecular surface of HRF dimer, supporting their role in interactions with IgE.

FIGS. 26A-26B: As shown in FIG. 25, HRF dimer, which can activate mast cells in an IgE-dependent manner, is formed by disulfide bonding between Cys172-Cys172 of two HRF monomers. Without being bound by theory, intracellular functions of HRF are carried out by the monomer, because the cytoplasm is a highly reducing milieu. Thus, it is presumed that the HRF locus with C172A mutation should encode mostly HRF monomer, which is bioactive as an intracellular protein. Indeed, HRF-C172A mice are healthy and breed well.

FIG. 27 shows that HRF-C172A mice are highly resistant to food allergy induction in an OVA-induced model. OVA-induced food allergy experiments were performed as described in FIG. 1 Body temperature and animal activity were monitored for 60 minutes after each OVA challenge.

FIG. 28 shows that resistance to food allergy induction in HRF-C172A mice strongly suggests that HRF dimer/HMW-HRFs play a crucial role in food allergy.

FIGS. 29A-29F show BALB/c mice were i.p. immunized with OVA plus alum and i.g. challenged with OVA (25 mg) three times a week. Immunized mice were either non-pretreated (OVA) or i.p. pretreated with 50 micrograms of Ba103 mAb or rat IgG (isotype, rIgG). Another control (Control) was non-immunized mice. (FIG. 29A) Procedure scheme. (FIG. 29B) The occurrence of diarrhea. p=0.0046 for isotype vs. Ba103; p=0.0024 for OVA vs. Ba103 by Log-rank test. (FIG. 29C) Basophil numbers in blood at day 29 were monitored by flow cytometry. ***, p<0.001 by Student's t-test. WT and Bas-TRECK BALB/c mice were i.p. immunized with OVA plus alum and i.g. challenged with OVA (25 mg) three times a week. Immunized mice were either i.p. pretreated with diphtheria toxin at day 28, 32, 36, 40 and 44 (1st treatment at 500 ng/mouse, other treatments at 250 ng/mouse). (FIG. 29D) Procedure scheme. (FIG. 29E) The occurrence of diarrhea. p=0.0017 for WT vs. Bas-TRECK by Log-rank test. (FIG. 29F) Basophil numbers in blood at day 29 (before 1st OVA challenge) were monitored by flow cytometry. Data shown as mean±SEM. **, p<0.01 by Mann-Whitney test.

FIG. 30: (Left) shows the crystal structure of recombinant human HRF-His6 dimer (Dore et al., Mol. Immunol., 93:216-222, 2018). The right panel shows heparinized blood from asthmatic, who was sensitized with house dust mite allergens, was incubated with PBS (no HRF), HRF dimer or monomer (10 micrograms/ml) in the presence of FITC-labeled anti-CD63 and APC-labeled anti-CCR3 at 37° C. for 15 min. After hemolysis, cells were analyzed by flow cytometry.

FIGS. 31A-31B illustrates passive cutaneous anaphylaxis (PCA) experiments showing significant differences between wild-type (WT) and HRF-C172A knock-in (KI) mice when stimulated with DNP₂-BSA, but not DNP₂₂-BSA. Mice were intradermally injected with anti-DNP IgE (right ear) or PBS (left ear). One day later, mice were challenged with intravenous injection of DNP₂₂-BSA (FIG. 31A) or DNP₂-BSA (FIG. 31B) (both at 0.1 mg/ml) in 1% Evans' blue dye. After 30 min, mice were sacrificed, and ears were cut and weighed, then digested overnight. Evans' blue dye was measured by spectrophotometer.

FIGS. 32A-32B shows passive systemic anaphylaxis (PSA) is less pronounced in HRF-C172A KI mice when antigen valency is low. Mice were intraperitoneally injected with anti-DNP IgE. One day later, mice were challenged with intravenous injection of DNP₂₂-BSA (FIG. 32A) or DNP₂-BSA (FIG. 32B) (0.1 mg/ml). Body temperature was monitored by an infrared thermometer for 60 min.

FIGS. 33A-33B show HRF dimers enhance IgE/antigen-induced activation of mast cells and basophils. FIG. 33A shows that mouse bone marrow-derived mast cells (BMMCs) were sensitized overnight with anti-TNP IgE C38-2, and then stimulated with indicated concentrations of TNP3-BSA together with mouse HRF (mHRF) dimer for 6 h. IL-13 in culture supernatants was quantified by ELISA. 50% effective concentrations (EC₅₀) of antigen were reduced by HRF dimer: 156 ng/ml for 0 μg/ml mHRF dimer; 124 ng/ml for 25 μg/ml mHRF dimer; 92 ng/ml for 50 μg/ml mHRF dimer. FIG. 33B shows heparinized blood from a house dust mite (HDM)-sensitized asthmatic was incubated with human HRF (hHRF) dimer (10 μg/ml in c) at 37° C. for 30 min. Then, HDM allergen (Dermatophagoides farinae at 10 ng/ml) was added to the cells together with FITC-anti-CD63 and APC-anti-CCR3 for 15 min. Expression of CD63 (activation marker) and CCR3 (basophil marker) was analyzed by flow cytometry. HRF monomers did not affect IgE/antigen-induced activation of mast cells or basophils (not shown).

### DETAILED DESCRIPTION

### Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. Thus, for example, reference to “an HRF sequence” or an “HRF binding antibody” includes a plurality of such HRF sequences or antibodies or subsequences thereof, and reference to “an HRF activity or function” can include reference to one or more HRF activities or functions, and so forth.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

A “subject” of diagnosis or treatment is a eukaryotic cell, a tissue culture, a tissue or an animal, e.g., a mammal, including a human and a juvenile. Non-human animals subject to diagnosis or treatment include, for example, a simian, a murine, a canine, a leporid, such as a rabbit, livestock, sport animals, and pets.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

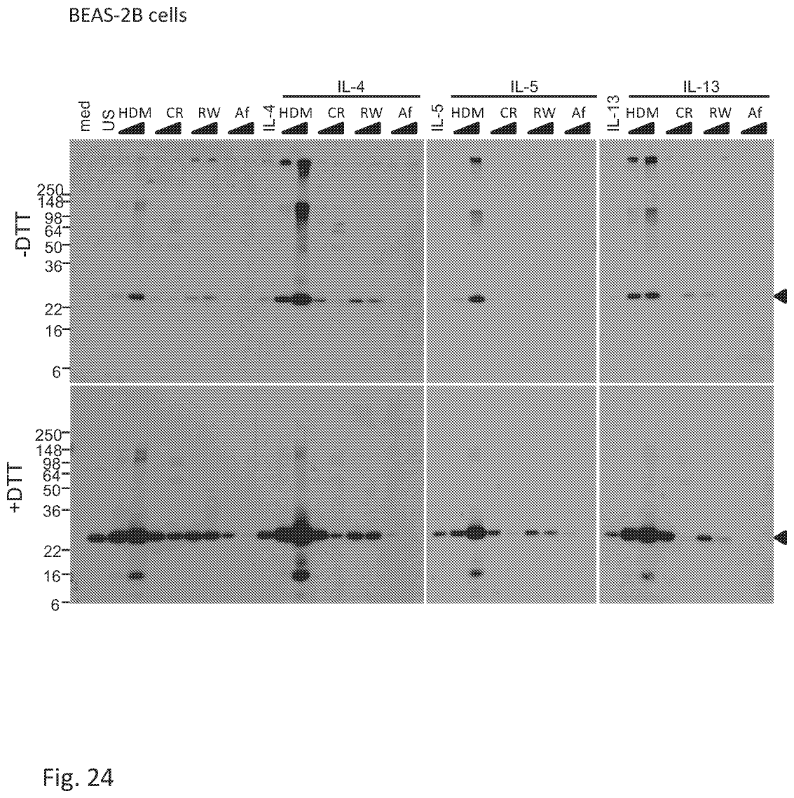

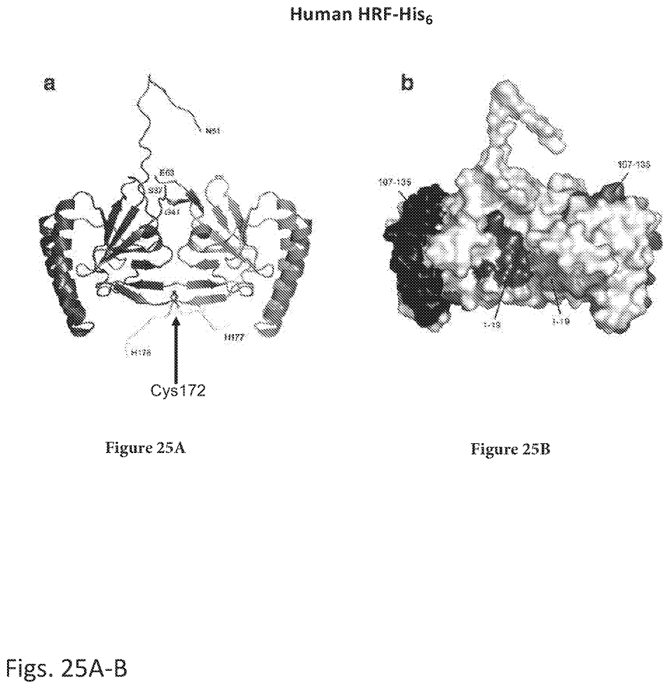

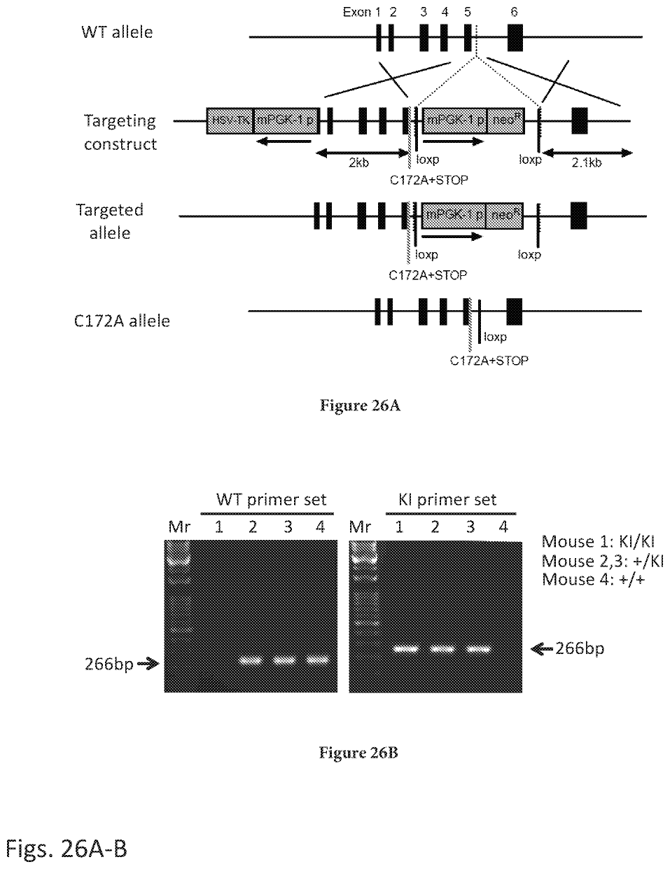

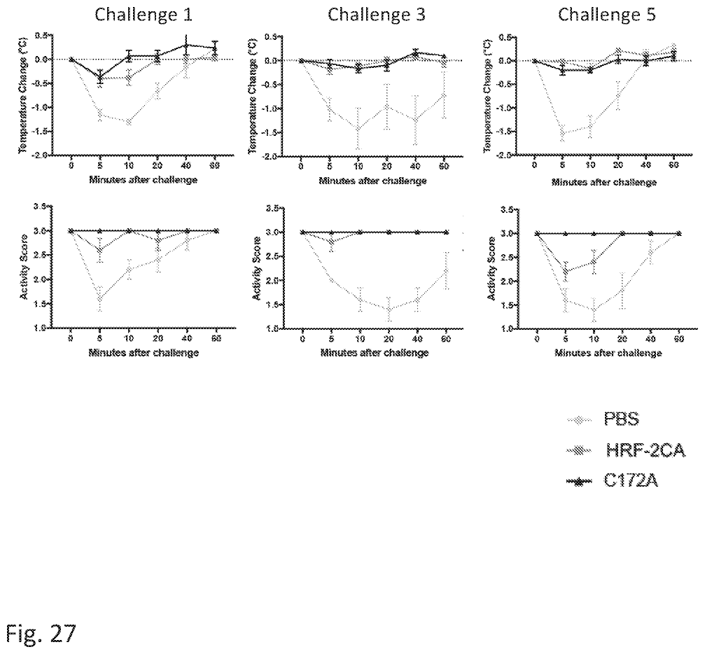

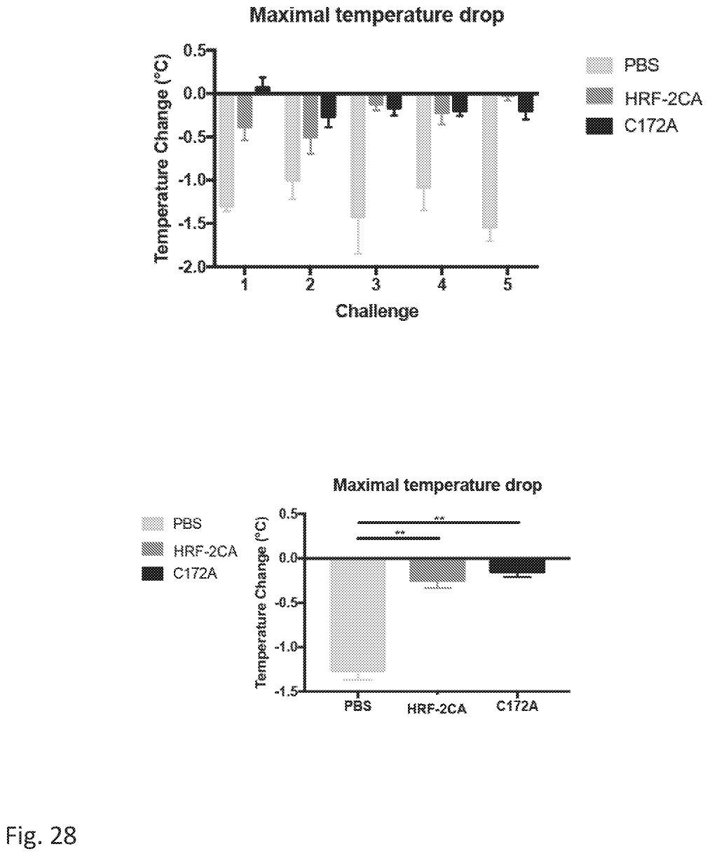

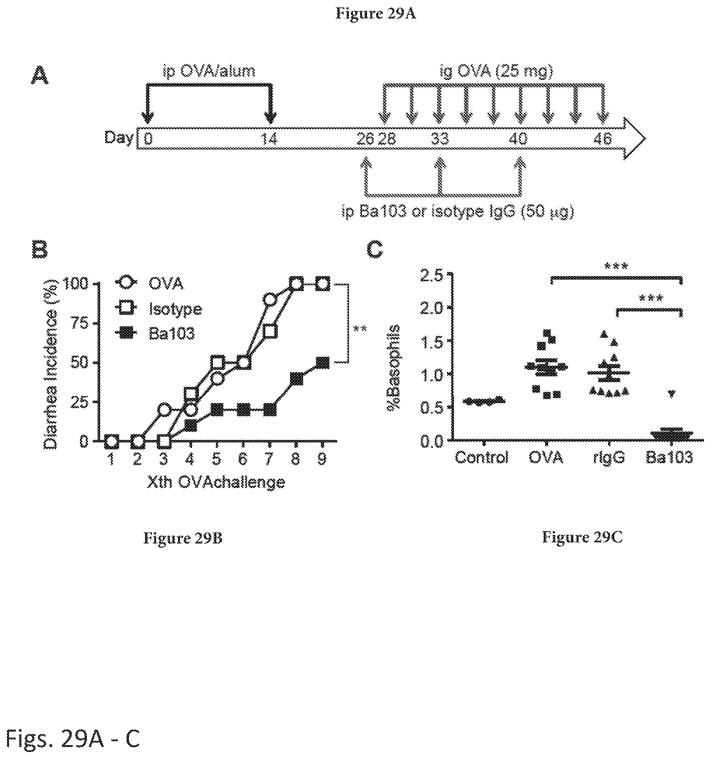

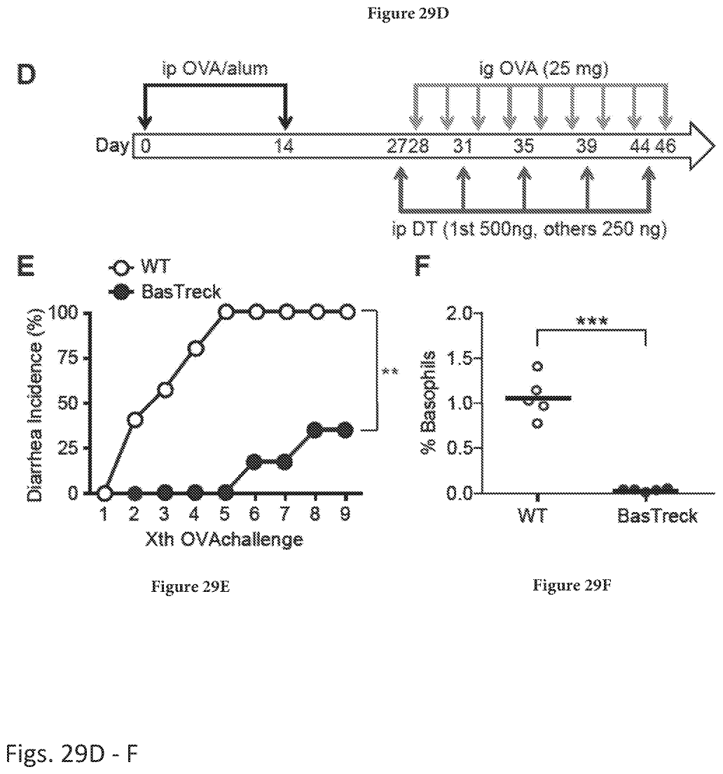

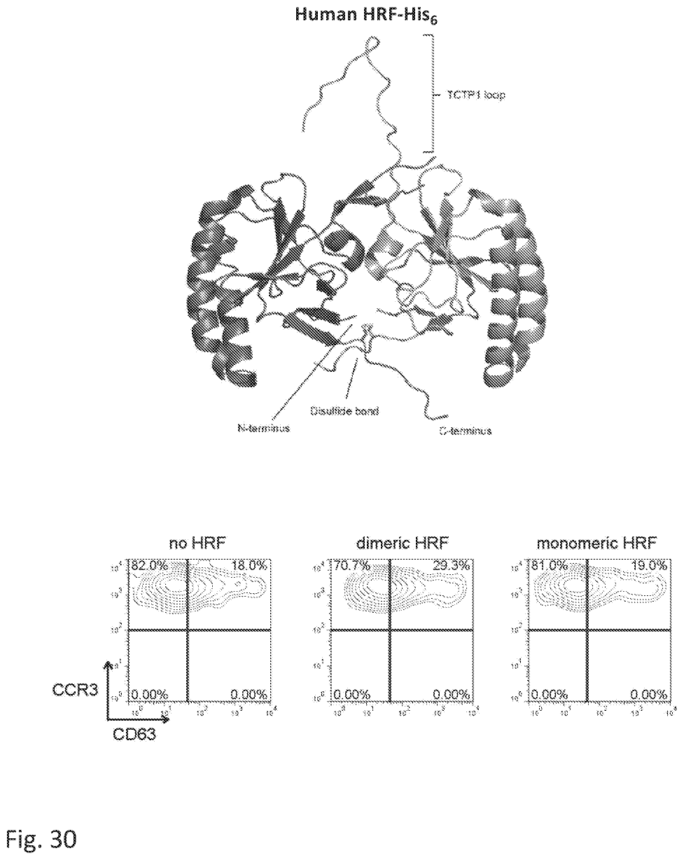

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. Clinical indications of treatment can include in some aspect, a reduction in diarrhea is an indication of success of treatment of a food allergy. Hypothermia and reduced physical activity (or mobility) are typical signs of anaphylaxis (which can be seen in food allergy and other allergic diseases) and can serve as a marker of allergic severity or a treatment. Thus, indication of normal temperature of increased physical activity are indications of successful treatment. Sub-clinical evidence of cytokines (see, e.g., FIG. 24) IL-4, IL-5, and IL-13, IL-25, IL-33, TSLP, IL-1beta, IL-6 and TNF, can be measured as an indication of disease severity and treatment.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

As used herein, the term “anti-histamine-releasing factor therapy” (anti-HRF therapy) comprises a peptide or polypeptide that that does one or more of inhibiting the binding of an HRF monomer, an HRF dimer, or an HRF multimer with an HRF-reactive immunoglobulin, or inhibiting HRF dimerization or HRF multimerization, or inhibiting HRF secretion or inhibiting cytokines that increase HRF secretion, or inhibiting FcεRI.

The anti-histamine-releasing factor therapy (anti-HRF therapy) of the present disclosure can be administered by any suitable route and may be practiced via systemic, regional or local administration, by any route. For example, an HRF sequence or an antibody that binds to HRF may be administered systemically, regionally or locally, via ingestion, via inhalation, topically, intravenously, orally (e.g., ingestion or inhalation), intramuscularly, intraperitoneally, intradermally, subcutaneously, intracavity, intracranially, transdermally (topical), parenterally, e.g. transmucosally or rectally. Compositions and methods of the disclosure including pharmaceutical formulations can be administered via a (micro) encapsulated delivery system or packaged into an implant for administration. It will also be appreciated that the preferred route will vary with the condition and age of the recipient, and the disease being treated. Methods of determining the most effective means and dosage of administration are known to those of skill in the art and will vary with the composition used for therapy, the purpose of the therapy, and the subject being treated. Single or multiple administrations can be carried out with the dose level and pattern being selected by the treating physician. Suitable dosage and methods of administering the agents are known in the art.

The anti-HRF therapy of the present disclosure can be used in the manufacture of medicaments and for the treatment of humans and other animals by administration in accordance with conventional procedures.

As used herein, the term “histamine-releasing factor-reactive immunoglobulin” (HRF-reactive Ig) refers to the subset of immunoglobulins that bind to HRF. In one aspect, HRF-reactive Ig refers to one or more of IgM, IgG, IgE, IgA, or IgD that bind to HRF.

As used herein, the term “sample” refers to a biological material isolated from a subject. In one aspect, sample refers to a fluid or lavage sample from a subject. In another aspect, sample comprises blood or plasma, body fluids, nasal fluids, tears or saliva. In a further aspect, sample comprises a biopsy of cells, tissue or organ.

As used herein, the term “initial antigen-specific Ig” refers to the subset of immunoglobulins that bind to the initial antigen or allergen. In one aspect, initial antigen-specific Ig refers to one or more of IgM, IgG, IgE, IgA, or IgD that bind to the initial antigen or allergen.

As used herein, the term “high levels of HRF-reactive IgE” refers to at least about 200 ng/ml, or alternatively at least about 225, or at least about 250, or at least about 300, or at least about 325, or at least about 350, or at least about 375, or at least about 400, or at least about 425, or at least about 450, all measured in ng per ml HE-1 IgE. RE-1 IgE is one of the two known human HRF-reactive IgEs.

As used herein, the term “low levels of HRF-reactive IgG” refers to no more than about 200 HG Unit mg/ml HRF-reactive IgG or an equivalent thereof as measured prior to the onset of allergic inflammation.

As used herein, the term “low levels of IgE” refers to no more than about 150 ng/ml, or alternative no more than about 125, or alternative no more than about 100, or alternative no more than about 75, or alternatively no more than about 50 ng per ml RE-1 IgE. RE-1 IgE is one of the two known human HRF-reactive IgEs.

As used herein, the term “high levels of HRF-dimerization or HRF-oligomerization” refers to at least about 500 AU HRF-dimers or at least about 300 ng/g tissue HRF multimers or an equivalent of each thereof as measured prior to the onset of allergic inflammation.

As used herein, the term “low levels of or initial antigen-specific IgE” refers to no more than about 20 kU_{A}/L initial antigen-specific IgE or an equivalent thereof as measured prior to the onset of allergic inflammation.

As used herein, the term “high levels of or initial antigen-specific IgE” refers to at least about 21 kU_{A}/L initial antigen-specific IgE or an equivalent thereof as measured prior to the onset of allergic inflammation.

As used herein, the term “high levels of HRF-reactive IgG” refers to at least about 201 HG Unit mg/ml HRF-reactive IgG or an equivalent thereof as measured prior to the onset of allergic inflammation.

As used herein, the term “low levels of HRF-dimerization or HRF-oligomerization” refers to no more than about 501 AU HRF-dimers or no more than about 301 ng/g tissue HRF multimers or an equivalent of each thereof as measured prior to the onset of allergic inflammation.

As used herein, the term “cytokines that increase HRF secretion” comprises Th2 cytokines (IL-4, IL-5 and IL-13), IL-1β, IL-6 and TNF, epithelial-derived cytokines such as TSLP, IL-25, and IL-33 that induce HRF secretion or an equivalent of each thereof.

As used herein, the term a “peptide or polypeptide that inhibits HRF dimerization or HRF multimerization” comprises peptides or polypeptides that can inhibit HRF from forming either dimers, oligomers, or multimers such as GST-tagged N-terminal 19 amino acids (GST-N19), or monomeric HRF (HRF-2CA) or an equivalent of each thereof.

As used herein, the term a “peptide or polypeptide that inhibits FcεRI” comprises a peptide or polypeptide that inhibits the activity of FcεRI such as Ectonucleotide pyrophosphatase-phosphodiesterase 3 (E-NPP3, also known as CD203c) or an equivalent thereof.

As used herein, the term an “oral immunotherapy” or “OTT” comprises, or alternatively consists essentially of, or yet further consists of at least two phases of ingestion of the allergen: (1) Phase 1 comprising upscaling patient exposure to allergen until desensitization is reached. In some cases, OTT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OTT comprises, or alternatively consists essentially of, or yet further consists of at least three phases of ingestion of the allergen: (1) phase 1 comprising an initial escalation phase with at least more than one dose of allergen within a single day, beginning with a small dose that is rapidly increased, non-limiting examples of this method include rush immunotherapy or cluster immunotherapy; (2) phase 2 comprises build-up dosing gradually under observation until a target dose is reached; and (3) phase 3 comprises daily maintenance dosing.

As used herein, the term “sublingual immunotherapy” (SLIT), comprises immunotherapy in which patients take gradually increased doses of allergen extract that are placed under the tongue and then spit or swallowed. In one aspect, SLIT protocols comprises two phases 1) phase 1 escalation phase comprising building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

As used herein, the term “epicutaneous immunotherapy” (EPIT) comprises immunotherapy with delivery of allergen to the skin through application of an allergen-containing patch designed to activate skin Langerhans cells, with subsequent migration to lymph nodes and downregulation of effector cell responses. Research has shown that mice sensitized to ovalbumin, peanut, or aeroallergens after 8 weeks of weekly EPIT treatment, showed decreased airway hyperreactivity with EPIT when subjected to allergen challenge (49). Only a few EPIT trials in humans have been conducted (50-52). A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

As used herein, the term “subcutaneous immunotherapy” (SCIT) comprises treating a subject with small doses of allergens administered subcutaneously or by injection. In one aspect, the allergen dose comprises about 3 μg to about 23 μg allergen per injection (54).

The disclosure is based, at least in part, on histamine releasing factor (HRF)/translationally controlled tumor protein (TCTP), and the identification of the HRF receptor (HRF-R) and inhibitors of HRF/HRF-R interactions. The disclosure is also based, at least in part, on identifying the role of HRF in food allergies, airway inflammation and skin or eye hypersensitivity.

In accordance with the disclosure, polypeptide (e.g., HRF, antibodies) sequences, such as substantially isolated, purified, and recombinant polypeptides, e.g., that bind to an immunoglobulin (Ig), are provided. In one embodiment, a polypeptide sequence is characterized as including or consisting of a subsequence of HRF (e.g., mammalian HRF) which binds to an immunoglobulin. In another embodiment, a polypeptide sequence is characterized as including or consisting of HRF amino acids 1-19 (e.g., MIIYRDLISHDEMFSDIYK (SEQ ID NO:1)) or HRF amino acids 79-142 (e.g., QETSFTKEAYKKYIKDYMKSIKGKLEEQRPERVKPFMTGAAEQIKHILANFKNYQFFIGENMNP (SEQ ID NO:2)), or an equivalent of each thereof, or a subsequence of HRF amino acids 1-19 or HRF amino acids 79-142 and equivalents of each thereof, and which subsequence binds to an immunoglobulin. In another embodiment, a polypeptide sequence is characterized as including or consisting of a subsequence of mammalian HRF/TCTP HRF amino acids MIIYRDLISHDEMFSDIYKIREIADGLCLEVEGKMVSRTEGNIDDSLIGGNASAEGPEGEGTE STVITGVDIVMNHHLQETSFTKEAYKKYIKDYMKSIKGKLEEQRPERVKPFMTGAAEQIKH ILANFKNYQFFIGENMNPDGMVALLDYREDGVTPYMIFFKDGLEMEKC (SEQ ID NO:3), or an equivalent thereof, where the sequence is not full length HRF, and has between about 5-171 HRF amino acid residues in length, and which sequence binds to an immunoglobulin. Non-limiting exemplary sequences less than full length HRF sequence include, for example, 5-10, 10-20, 20-30, 30-40, 40-50, 50-75, 75-100, 100-150, and 150-171 amino acid residues of HRF sequence.

Exemplary mammalian HRF sequences include human and non-human HRF sequences. Exemplary Human (NM_003295), Mouse (NM_009429), Rat (NM_053867), Rabbit (NM_001082129), Guinea Pig (NM_001173082), Chimpanzee (NM_001098546), Monkey (NM_001095869), Dog (NM_851473), Pig (NM_214373), Bovine (NM_001014388), respectively, are set forth, in order, in Table 1 (SEQ ID NOs: 4-13, listed below).

TABLE 1

| | |
|---|---|
| 1:MITYRDLISHDEMFSDIYKIREIADGLCLEVEGKMVSRTEGNIDDSLIGGNASAEGPEGE | 60 |
| 1:MITYRDLISHDELFSDIYKIREIADGLCLEVEGKMVSRTEGAIDDSLIGGNASAEGPEGE | 60 |
| 1:MITYRDLISHDELFSDIYKIREIADGLCLEVEGKMVSRTEGAIDDSLIGGNASAEGPEGE | 60 |
| 1:MITYRDLISHDEMFSDIYKIREIAGGLCLEVEGKMVSRTEGNIDDSLIGGNASAEGPEGE | 60 |
| 1:MITYRDLISHDEMFSDIYKIREIADGLCLEVEGKMVSRTEGNIDDSLIGGNASAEGPEGE | 60 |
| 1:MITYRDLISHDEMFSDIYKIREIADGLCLEVEGKMVSRTEGNIDDSLIGGNASAEGPEGE | 60 |
| 1:MITYRDLISHDEMFSDIYKIREIADGLCLEVEGK-VSRTEGNIDDSLIGGNASAEGPEGE | 60 |
| 1:MITYRDLISHDEMFSDIYKIREIADGLCLEVEGKMVSRTEGNIDDSLIGGNASAEGPEGE | 60 |
| 1:MITYRDLISHDEMFSDIYKIREIADGLCLEVEGKMVSRTEGNIDDSLIGGNASAEGPEGE | 60 |
| 1:MITYRDLISHDEMFSDIYKIREVADGLCLEVEGKMVSRTEGNIDDSLIGGNASAEGPEGE | 60 |
| 61:GTESTVITGVDIVMNHHLQETSFTKEAYKKYIKDYMKSIKGKLEEQRPERVKPFMTGAAE | 120 |
| 61:GTESTVVTGVDIVMNHHLQETSFTKEAYKKYIKDYMKSLKGKLEEQKPERVKPFMTGAAE | 120 |
| 61:GTESTVVTGVDIVMNHHLQETSFTKEAYKKYIKDYMKSLKGKLEEQKPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 61:GTESTVITGVDIVMNHHLQETSFTKEAYKKYIKDYMKSIKGKLEEQRPERVKPFMTGAAE | 120 |
| 121:QIKHILANFKNYQFFIGENMNPDGMVALLDYREDGVTPYMIFFKDGLEMEKC | 172 |
| (SEQ ID NO. 4) | |
| 121:QIKHILANFNNYQFFIGENMNPDGMVALLDYREDGVTPFMIFFKDGLEMEKC | 172 |
| (SEQ ID NO. 5) | |
| 121:QIKHILANFNNYQFFIGENMNPDGMVALLDYREDGVTPFMIFFKDGLEMEKC | 172 |
| (SEQ ID NO. 6) | |
| 121:QIKHILANFKNYQFYIGENMNPDGMVALLDYREDGVTPFMIFFKDGLEMEKC | 172 |
| (SEQ ID NO. 7) | |
| 121:QIKHILANFKNYQFFIGANMNPDGMVALLDYREDGVTPFMIFFKDGLEMEKC | 172 |
| (SEQ ID NO. 8) | |
| 121:QIKHILANFKNYQFFIGENMNPDGMVALLDYREDGVTPYMIFFKDGLEMEKC | 172 |
| (SEQ ID NO. 9) | |
| 121:QIKHILANFKNYQFFIGENMNPDGMVALLDYREDGVTPYMIFFKDGLEMEKC | 172 |
| (SEQ ID NO. 10) | |
| 121:QIKHILANFKNYQFFIGENMNPDGMVALLDYREDGVTPYMIFFKDGLEMEKC | 172 |
| (SEQ ID NO. 11) | |
| 121:QIKHILANFKNYQFFIGENMNPDGMVALLDYREDGVTPYMIFFKDGLEMEKC | 172 |
| (SEQ ID NO. 12) | |
| 121:QIKHILANFKNYQFFIGENMNPDGMVALLDYREDGVTPYMIFFKDGLEMEKC | 172 |
| (SEQ ID NO. 13) | |

Exemplary HRF sequences that bind to an immunoglobulin (Ig) include HRF that binds to one or more of IgM, IgG, IgE, IgA, or IgD. Particular IgE to which HRF binds are associated with immune disorders and diseases such as those associated with allergies (food or other antigens), asthma, hypersensitivity reactions and inflammation.

As used herein, the terms “peptide,” “polypeptide” and “protein” are used interchangeably and refer to two or more amino acids covalently linked by an amide bond or equivalent. The polypeptides of the disclosure are of any length and include L- and D-isomers, and combinations of L- and D-isomers. The polypeptides can include modifications typically associated with post-translational processing of proteins, for example, cyclization (e.g., disulfide bond), phosphorylation, glycosylation, carboxylation, ubiquitination, myristylation, acetylation (N-terminal), amidation (C-terminal), or lipidation. Polypeptides described herein further include compounds having amino acid structural and functional analogues, for example, peptidomimetics having synthetic or non-natural amino acids or amino acid analogues, so long as the mimetic has one or more functions or activities of a native polypeptide set forth herein. Non-natural and non-amide chemical bonds, and other coupling means can also be included, for example, glutaraldehyde, N-hydoxysuccinimide esters, bifunctional maleimides, or N,N′-dicyclohexylcarbodiimide (DCC). Non-amide bonds can include, for example, ketomethylene aminomethylene, olefin, ether, thioether and the like (see, e.g., Spatola (1983) in Chemistry and Biochemistry of Amino Acids, Peptides and Proteins, Vol. 7, pp 267-357, “Peptide and Backbone Modifications,” Marcel Decker, N.Y.).

As used herein, the term “equivalent” in the context of describing a peptide, a polypeptide, or a protein encompass the wild-type sequence, sequences of variants, and sequences comprising one or more modifications. For example, an equivalent of a HRF monomer of SEQ ID NO: 4 can encompass a HRF variant such as those listed in Table 1. An equivalent of a HRF monomer of SEQ ID NO: 4 can also encompass at least 50%, 60%, 70%, 80%, 90%, 91%, 92%, 93%, 94%, 95%, 96%, 97%, 98%, or 99% sequence identity to SEQ ID NO: 4. An equivalent of a HRF monomer of SEQ ID NO: 4 can further encompass at least 50%, 60%, 70%, 80%, 90%, 91%, 92%, 93%, 94%, 95%, 96%, 97%, 98%, or 99% sequence identity to SEQ ID NO: 4 while retaining one or more modifications, optionally amino acid substitution(s) at C28 and/or C172 according to SEQ ID NO: 4.

The term “isolated,” when used as a modifier of a composition (e.g., HRF sequences, antibodies, subsequences, modified forms, nucleic acids encoding same, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., an HRF sequence or antibody) can also be “substantially pure” or “purified” when free of most or all of the materials with which it typically associates with in nature. Thus, an isolated sequence that also is substantially pure or purified does not include polypeptides or polynucleotides present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of HRF sequences or antibodies, subsequences, and other antibodies, agents, drugs or therapies.

As used herein, the term “recombinant,” when used as a modifier of polypeptides, polynucleotides and antibodies, means that the compositions have been manipulated (i.e., engineered) in a fashion that generally does not occur in nature (e.g., in vitro). A particular example of a recombinant polypeptide would be where an HRF polypeptide or antibody is expressed by a cell transfected with a polynucleotide encoding the HRF polypeptide or antibody sequence. A particular example of a recombinant polynucleotide would be where a nucleic acid (e.g., genomic or cDNA) encoding HRF cloned into a plasmid, with or without 5′, 3′ or intron regions that the gene is normally contiguous with in the genome of the organism. Another example of a recombinant polynucleotide or polypeptide is a hybrid or fusion sequence, such as a chimeric HRF or antibody sequence comprising and a second sequence, such as a heterologous functional domain.

The disclosure also provides antibodies and subsequences thereof which are useful to bind to or that modulate an HRF activity or function, or HRF expression. The term “antibody” refers to a protein that binds to other molecules (antigens) via heavy and light chain variable domains, V_{H} and V_{L}, respectively. Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

In accordance with the disclosure, there are provided antibodies and subsequences thereof that bind to a HRF/TCTP sequence that includes or consists of a region of HRF that binds to an Ig, such as an IgE. In a particular embodiment, a sequence of HRF to which antibodies or subsequences thereof bind include or consist of amino acids 1-19 (MIIYRDLISHDEMFSDIYK (SEQ ID NO:1)) or amino acids 79-142 (QETSFTKEAYKKYIKDYMKSIKGKLEEQRPERVKPFMTGAAEQIKHILANFKNYQFFIGENMNP (SEQ ID NO:2)) of mammalian HRF, or an equivalent of each thereof. Such antibodies can also bind to any subsequence of the HRF/TCTP sequence that includes or consists of a region of HRF that binds to an Ig, such as an IgE. In a particular embodiment, a subsequence is a portion of amino acids 1-19 (MIIYRDLISHDEMFSDIYK (SEQ ID NO:1)) or a portion of amino acids 79-142 (QETSFTKEAYKKYIKDYMKSIKGKLEEQRPERVKPFMTGAAEQIKHILANFKNYQFFIGENMNP (SEQ ID NO:2)) of mammalian HRF, or a portion of MIIYRDLISHDEMFSDIYKIREIADGLCLEVEGKMVSRTEGNIDDSLIGGNASAEGPEGEGTE STVITGVDIVMNHHRLQETSFTKEAYKKYIKDYMKSIKGKLEEQRPERVKPFMTGAAEQIKH ILANFKNYQFFIGENMNPDGMVALLDYREDGVTPYMIFFKDGLEMEKC (SEQ ID NO:3), wherein the subsequence is between 5-171 amino acid residues in length, e.g., 5-10, 10-20, 20-50, 100-150, or 150-171 amino acid residues in length, or an equivalent of each thereof.

The term “bind,” or “binding,” when used in reference to an HRF sequence or antibody, means that the HRF sequence, antibody or subsequence thereof interacts at the molecular level with an Ig, such as an IgE, or a corresponding epitope (antigenic determinant) present on HRF, respectively. Thus, an HRF binds to all or a part of an Ig sequence, and an antibody specifically binds to all or a part of sequence or an antigenic epitope on HRF (e.g., an HRF region that confers binding to an Ig, such as an IgE). Specific binding is that which is selective for the Ig or HRF. Antibodies and subsequences thereof include specific or selective binding to HRF, particularly a region or an epitope within HRF amino acids 1-19 (MIIYRDLISHDEMFSDIYK (SEQ ID NO. 1)) or HRF amino acids 79-142 (QETSFTKEAYKKYIKDYMKSIKGKLEEQRPERVKPFMTGAAEQIKHILANFKNYQFFIGENMNP (SEQ ID NO. 2)), or an equivalent of each thereof. Specific and selective binding can be distinguished from non-specific binding using assays known in the art (e.g., competition binding, immunoprecipitation, ELISA, flow cytometry, Western blotting).

Antibodies of the disclosure and disclosure methods employing antibodies include polyclonal and monoclonal antibodies. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies of the disclosure and disclosure methods employing antibodies can belong to any antibody class, IgM, IgG, IgE, IgA, IgD, or subclass. Exemplary subclasses for IgG are IgG₁, IgG₂, IgG₃ and IgG₄.

Antibodies of the disclosure and disclosure methods employing antibodies include antibody subsequences and fragments. Exemplary antibody subsequences and fragments include Fab, Fab′, F(ab′)₂, Fv, Fd, single-chain Fv (scFv), disulfide-linked Fvs (sdFv), light chain variable region V_{L}, heavy chain variable region V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, (scFv)₂-Fc and IgG4PE. Such subsequences and fragments can have the binding affinity as the full length antibody, the binding specificity as the full length antibody, or one or more activities or functions of as a full length antibody, e.g., a function or activity of HRF binding antibody.

Antibody subsequences and fragments can be combined. For example, a V_{L} or V_{H} subsequences can be joined by a linker sequence thereby forming a V_{L}-V_{H} chimera. A combination of single-chain Fvs (scFv) subsequences can be joined by a linker sequence thereby forming a scFv-scFv chimera. Antibody subsequences and fragments include single-chain antibodies or variable region(s) alone or in combination with all or a portion of other subsequences.

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)2. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enymol. 1:422 (1967)). Other methods of cleaving antibodies, such as separation of heavy chains to form monovalent light-heavy chain fragments, further cleavage of fragments, or other enzymatic or chemical may also be used.

Epitopes typically are short amino acid sequences, e.g. about five to 15 amino acids in length. Epitopes can be contiguous or non-contiguous. A non-contiguous amino acid sequence epitope forms due to protein folding. For example, an epitope can include a non-contiguous amino acid sequence, such as a 5 amino acid sequence and an 8 amino acid sequence, which are not contiguous with each other, but form an epitope due to protein folding. Techniques for identifying epitopes are known to the skilled artisan and include screening overlapping oligopeptides for binding to antibody (for example, U.S. Pat. No. 4,708,871), phage display peptide library kits, which are commercially available for epitope mapping (New England BioLabs). Epitopes may also be identified by inference when epitope length peptide sequences are used to immunize animals from which antibodies that bind to the peptide sequence are obtained and can be predicted using computer programs, such as BEPITOPE (Odorico et al., J. Mol. Recognit. 16:20 (2003)).

Methods of producing polyclonal and monoclonal antibodies are known in the art. For example, HRF, or a subsequence thereof, or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using conventional hybridoma technology, splenocytes from immunized animals that respond to HRF can be isolated and fused with myeloma cells. Monoclonal antibodies produced by the hybridomas can be screened for reactivity with HRF or an immunogenic fragment thereof.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of HRF, or a subsequence thereof, preparation, and may be at regular or irregular intervals.

Human antibodies can be produced by immunizing human transchromosomic KM Mice™ (WO 02/43478) or HAC mice (WO 02/092812). KM Mice™ and HAC mice express human immunoglobulin genes. Using conventional hybridoma technology, splenocytes from immunized mice that were high responders to the antigen can be isolated and fused with myeloma cells. A monoclonal antibody can be obtained that binds to the antigen. An overview of the technology for producing human antibodies is described in Lonberg and Huszar (Int. Rev. Immunol. 13:65 (1995)). Transgenic animals with one or more human immunoglobulin genes (kappa or lambda) that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Antibodies of the disclosure and disclosure methods employing antibodies include mammalian, primatized, humanized, fully human antibodies and chimeras. A mammalian antibody is an antibody produced by a mammal, transgenic or non-transgenic, or a non-mammalian organism engineered to produce a mammalian antibody, such as a non-mammalian cell (bacteria, yeast, insect cell), animal or plant.

The term “human” when used in reference to an antibody, means that the amino acid sequence of the antibody is fully human, i.e., human heavy and human light chain variable and human constant regions. Thus, all of the amino acids are human or exist in a human antibody. An antibody that is non-human may be made fully human by substituting the non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk (1987). A consensus sequence of human V_{H} subgroup III, based on a survey of 22 known human V_{H} III sequences, and a consensus sequence of human V_{L} kappa-chain subgroup I, based on a survey of 30 known human kappa I sequences is described in Padlan Mol. Immunol. 31:169 (1994); and Padlan Mol. Immunol. 28:489 (1991). Human antibodies therefore include antibodies in which one or more amino acid residues have been substituted with one or more amino acids present in any other human antibody.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Such antibodies typically have reduced immunogenicity and therefore a longer half-life in humans as compared to the non-human parent antibody from which one or more CDRs were obtained or are based upon.

Antibodies of the disclosure and disclosure methods employing antibodies include those to as “primatized” antibodies, which are “humanized” except that the acceptor human immunoglobulin molecule and framework region amino acid residues may be any primate amino acid residue (e.g., ape, gibbon, gorilla, chimpanzees orangutan, macaque), in addition to any human residue. Human FR residues of the immunoglobulin can be replaced with corresponding non-human residues. Residues in the CDR or human framework regions can therefore be substituted with a corresponding residue from the non-human CDR or framework region donor antibody to alter, generally to improve, antigen affinity or specificity, for example. A humanized antibody may include residues, which are found neither in the human antibody nor in the donor CDR or framework sequences. For example, a FR substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., Nature 332:323 (1988)).

The term “chimeric” and grammatical variations thereof, when used in reference to an antibody, means that the amino acid sequence of the antibody contains one or more portions that are derived from, obtained or isolated from, or based upon two or more different species. For example, a portion of the antibody may be human (e.g., a constant region) and another portion of the antibody may be non-human (e.g., a murine heavy or murine light chain variable region). Thus, an example of a chimeric antibody is an antibody in which different portions of the antibody are of different species origins. Unlike a humanized or primatized antibody, a chimeric antibody can have the different species sequences in any region of the antibody.

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Suitable techniques that additionally may be employed in antibody methods include affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

HRF or a subsequence thereof, suitable for generating antibodies can be produced by any of a variety of standard protein purification or recombinant expression techniques known in the art. For example, HRF or a subsequence thereof can be recombinantly produced or obtained from cells.

Forms of protein suitable for generating an immune response include peptide subsequences of full length protein, such as an immunogenic fragment. Additional forms of protein include preparations or cell extracts or fractions, partially purified HRF or a subsequence thereof, as well as whole cells that express HRF, or a subsequence thereof, or preparations of HRF or a subsequence thereof, expressing cells.

Proteins and antibodies, as well as subsequences and fragments thereof, can be produced by genetic methodology. Such techniques include expression of all or a part of the gene encoding the protein or antibody into a host cell such as Cos cells or E. coli. The recombinant host cells synthesize full length or a subsequence, for example, an scFv (see, e.g., Whitlow et al., In: Methods: A Companion to Methods in Enzymology 2:97 (1991), Bird et al., Science 242:423 (1988); and U.S. Pat. No. 4,946,778). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988).

In accordance with the disclosure, also provided are modified forms of proteins, antibodies, nucleic acids, and other compositions, provided that the modified form retains, at least a part of, a function or activity of the unmodified or reference protein, nucleic acid, or antibody. For example, a modified HRF (e.g., a subsequence or fragment) can retain at least partial binding to an Ig, such as an IgE. In another non-limiting example, a modified HRF (e.g., a subsequence or fragment) can be used as an immunogen to produce antibodies that specifically bind to HRF. In yet another non-limiting example, a modified HRF or HRF binding antibody (e.g., a subsequence or fragment) can be used in any disclosure method.

The disclosure therefore includes modified forms of proteins, antibodies, nucleic acids, and other compositions. Such modified forms typically retain, at least a part of, one or more functions or activities of an unmodified or reference protein, nucleic acid, or antibody. Such activities include, for example, for HRF, binding to a receptor, such as an Ig, such as an IgE, or modulating HRF activity, function or expression, etc., and for an HRF antibody, binding to HRF and inhibiting interactions between HRF and Igs, such as IgE.

As used herein, the term “modify” and grammatical variations thereof, means that the composition deviates from a reference composition. Such modified proteins, nucleic acids and other compositions may have greater or less activity or function, or have a distinct function or activity compared with a reference unmodified protein, nucleic acid, or composition.

A “functional polypeptide” or “active polypeptide” refers to a modified polypeptide that possesses at least one partial function or biological activity characteristic of a native wild type or full length counterpart polypeptide as described herein, which can be identified through an assay. As described herein, a particular example of a biological activity of HRF is to bind to an Ig, such as an IgE. Another example of a biological activity is the ability of an antibody to bind to HRF sequences, such as antibody fragments that can bind to HRF, such as the sequence region of HRF that confers binding to an Ig, such as an IgE. Yet another example is an HRF subsequence that modulates (e.g., decrease, reduce, inhibit, suppress, limit or control) native (endogenous) HRF activity, function or expression in vitro, ex vivo or in vivo, presumably by binding to HRF-R which in turn limits activity or function or downstream signaling that occurs between native HRF and native HRF-R.

Modifications include, for example, substitutions, additions, insertions and deletions to the amino acid sequences set forth herein, which can be referred to as “variants.” Exemplary sequence substitutions, additions, and insertions include a full length or a portion of a sequence with one or more amino acids substituted (or mutated), added, or inserted, for example of an HRF sequence, wherein the modified HRF binds to an Ig, such as an IgE, or of an antibody that binds to HRF, such as the sequence region of HRF that confers binding to an Ig, such as an IgE.

Modified polypeptides include, for example, non-conservative and conservative substitutions of the HRF or antibody amino acid sequences. In particular embodiments, a modified protein has one or a few (e.g., 10-20% of the residues of total protein length, or 2-10 residues, substituted) conservative or non-conservative substitutions.

As used herein, the term “conservative substitution” denotes the replacement of an amino acid residue by another, chemically or biologically similar residue. Biologically similar means that the substitution does not destroy a biological activity or function, e.g., HRF binding activity to an Ig, or antibody binding to HRF. Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

Modified forms of protein (e.g., HRF, HRF fragment or antibody), nucleic acid, and other compositions include additions and insertions, such as of heterologous domains. For example, an addition (e.g., heterologous domain) can be the covalent or non-covalent attachment of any type of molecule to a protein (e.g., HRF, HRF fragment or HRF antibody), nucleic acid or other composition. Typical additions and insertions (e.g., a heterologous domain) confer a complementary or a distinct function or activity.

In some embodiments, modified forms of a HRF molecule comprises a modified HRF monomer, a modified HRF dimer, or a modified HRF multimer. In some instances, a modified HRF molecule comprises one or more substitutions (or mutations). In some cases, the modified HRF molecule comprises a full-length HRF protein or a fragment thereof (e.g., a functional fragment that is capapble of binding to a HRF-reactive Ig). In some cases, the parent sequence from which the modified HRF molecule is derived from is a wild-type HRF protein (optionally comprising SEQ ID NO: 4 as disclosed in WO 2020/102108, published May 22, 2020), or a variant thereof (optionally, HRF isoform 1 (NP_001273201.1), HRF isoform 2 (NP_003286.1), or HRF isoform 3 (NP_001273202.1)). As utilized herein, the term “HRF” and “HRF molecule” are used interchangeably and refer to a HRF monomer, a HRF dimer, or a HRF multimer.

In some instances, a modified HRF molecule comprises a substitution (or mutation) at amino acid residue C28, in which the position corresponds to position 28 of SEQ ID NO: 4 as disclosed in WO 2020/102108, published May 22, 2020. The mutation can be a conservative substitution. The mutation can also be a non-conservative mutation. The mutation can be a substitution to a positively charged amino acid (e.g., Arg, His, or Lys) or to a negatively charged amino acid (e.g., Asp or Glu). The mutation can be a substitution to a polar amino acid (e.g., Ser, Thr, Asn, or Gln) or a hydrophobic amino acid (e.g., Ala, Val, Ile, Leu, Met, Phe, Tyr, Trp, or Pro). The mutation can be a substitution to Gly. The mutation can be a substitution to Ala. The modified HRF molecule can be a modified HRF monomer, a modified HRF dimer, or a modified HRF multimer. The modified HRF molecule can comprise at least one HRF monomer those sequence is an equivalent thereof of SEQ ID NO: 4 as disclosed in WO 2020/102108, published May 22, 2020, in which the equivalent comprises at least 70% (optionally, 80%, 85%, 90%, 91%, 92%, 93%, 94%, 95%, 96%, 97%, 98%, 99%, or 99.5%) sequence identity to SEQ ID NO: 4, while retaining the amino acid substitution at C28 according to SEQ ID NO: 4 as disclosed in WO 2020/102108, published May 22, 2020.

In some instances, a modified HRF molecule comprises a substitution (or mutation) at amino acid residue C172, in which the position corresponds to position 172 of SEQ ID NO: 4 as disclosed in WO 2020/102108, published May 22, 2020. The mutation can be a conservative substitution. The mutation can also be a non-conservative mutation. The mutation can be a substitution to a positively charged amino acid (e.g., Arg, His, or Lys) or to a negatively charged amino acid (e.g., Asp or Glu). The mutation can be a substitution to a polar amino acid (e.g., Ser, Thr, Asn, or Gln) or a hydrophobic amino acid (e.g., Ala, Val, Ile, Leu, Met, Phe, Tyr, Trp, or Pro). The mutation can be a substitution to Gly. The mutation can be a substitution to Ala. The modified HRF molecule can be a modified HRF monomer, a modified HRF dimer, or a modified HRF multimer. The modified HRF molecule can comprise at least one HRF monomer those sequence is an equivalent thereof of SEQ ID NO: 4 as disclosed in WO 2020/102108, published May 22, 2020, in which the equivalent comprises at least 70% (optionally, 80%, 85%, 90%, 91%, 92%, 93%, 94%, 95%, 96%, 97%, 98%, 99%, or 99.5%) sequence identity to SEQ ID NO: 4 as disclosed in WO 2020/102108, published May 22, 2020, while retaining the amino acid substitution at C172 according to SEQ ID NO: 4 as disclosed in WO 2020/102108, published May 22, 2020.

In some cases, a modified HRF molecule comprises two or more modifications. For example, the modified HRF molecule can be a modified HRF monomer comprising two or more modifications. The modified HRF molecule can be a modified HRF dimer comprising a modification in each monomer, or one or more modifications in each monomer. The modified HRF molecule can further be a modified multimer comprising a modification in each monomer unit, or one or more modifications in each monomer unit. In some cases, the modification comprises a substitution at amino acid residue C28, or a combination thereof, in which the position corresponds to position 28 of SEQ ID NO: 4 as disclosed in WO 2020/102108, published May 22, 2020. Optionally, the modification at C28 is a substitution to Ala. The modified HRF molecule can comprise at least one HRF monomer those sequence is an equivalent thereof of SEQ ID NO: 4, in which the equivalent comprises at least 70% (optionally, 80%, 85%, 90%, 91%, 92%, 93%, 94%, 95%, 96%, 97%, 98%, 99%, or 99.5%) sequence identity to SEQ ID NO: 4 as disclosed in WO 2020/102108, published May 22, 2020, while retaining the amino acid substitution at C28 according to SEQ ID NO: 4 as disclosed in WO 2020/102108, published May 22, 2020.

In some cases, a modified HRF molecule comprises two or more modifications. For example, the modified HRF molecule can be a modified HRF monomer comprising two or more modifications. The modified HRF molecule can be a modified HRF dimer comprising a modification in each monomer, or one or more modifications in each monomer. The modified HRF molecule can further be a modified multimer comprising a modification in each monomer unit, or one or more modifications in each monomer unit. In some cases, the modification comprises a substitution at amino acid residue C172, or a combination thereof, in which the position corresponds to position 172 of SEQ ID NO: 4 as disclosed in WO 2020/102108, published May 22, 2020. Optionally, the modification at C172 is a substitution to Ala. The modified HRF molecule can comprise at least one HRF monomer those sequence is an equivalent thereof of SEQ ID NO: 4, in which the equivalent comprises at least 70% (optionally, 80%, 85%, 90%, 91%, 92%, 93%, 94%, 95%, 96%, 97%, 98%, 99%, or 99.5%) sequence identity to SEQ ID NO: 4 as disclosed in WO 2020/102108, published May 22, 2020, while retaining the amino acid substitution at C172 according to SEQ ID NO: 4 as disclosed in WO 2020/102108, published May 22, 2020.

In some cases, a modified HRF molecule comprises two or more modifications. For example, the modified HRF molecule can be a modified HRF monomer comprising two or more modifications. The modified HRF molecule can be a modified HRF dimer comprising a modification in each monomer, or one or more modifications in each monomer. The modified HRF molecule can further be a modified multimer comprising a modification in each monomer unit, or one or more modifications in each monomer unit. In some cases, the modification comprises a substitution at amino acid residue C28 and C172, optionally in combination with one or more additional substitutions, in which the positions correspond to positions 28 and 172 of SEQ ID NO: 4 as disclosed in WO 2020/102108, published May 22, 2020. Optionally, the modification at C28 and/or C172 is a substitution to Ala. The modified HRF molecule can comprise at least one HRF monomer those sequence is an equivalent thereof of SEQ ID NO: 4 as disclosed in WO 2020/102108, published May 22, 2020, in which the equivalent comprises at least 70% (optionally, 80%, 85%, 90%, 91%, 92%, 93%, 94%, 95%, 96%, 97%, 98%, 99%, or 99.5%) sequence identity to SEQ ID NO: 4, while retaining the amino acid substitutions at C28 and C172 according to SEQ ID NO: 4 as disclosed in WO 2020/102108, published May 22, 2020.

In some instances, the modified HRF molecule comprises a HRF monomer that comprises at least 70% sequence identity to SEQ ID NO: 4 as disclosed in WO 2020/102108, published May 22, 2020 while still comprising the amino acid substitution(s) at C28 and/or C172 according to SEQ ID NO: 4 as disclosed in WO 2020/102108, published May 22, 2020. In some cases, the HRF monomer comprises at least 80%, or alternatively 85%, or alternatively at least 90%, or alternatively at least 96%, or alternatively at least 97%, or alternatively at least 98%, or alternatively at least 99%, or alternatively at least 99.5%, sequence identity to SEQ ID NO: 4 as disclosed in WO 2020/102108, published May 22, 2020, each while still comprising the amino acid substitution(s) at C28 and/or C172 according to SEQ ID NO: 4 as disclosed in WO 2020/102108, published May 22, 2020, that optionally comprise the modification at C28 and/or C172 to Ala.

In some embodiments, one or more of the HRF molecule is a bioactive (or biologically active) molecule. In some instances, the HRF dimer is bioactive or biologically active.

In some embodiments, one or more of the HRF molecule is not a bioactive (or biologically active) molecule. In some instances, the HRF monomer is not bioactive or biologically active.

In some cases, the two HRF monomers within a HRF dimer interact with each other through non-covalent interactions (e.g., ionic interaction such as a salt-bridge interaction, hydrogen bonding, Van der Waals interaction, hydrophobic interaction, or a combination thereof), a covalent interaction (e.g., a disulfide bond), or a combination thereof. In some instances, the HRF dimer comprises a disulfide bond formed between the HRF monomers. In some cases, the disulfide bond is formed by the C172 residue within each of the HRF monomer. In some cases, the dimeric interface comprises Met1, Ile17, residues 34-39, Met74, Asn75 from a first HRF monomer and Met1, Ile3, Glu12, residues 33-37, Asn75, and His76 from a second HRF monomer. In some cases, a HRF dimer is as described in Dore, et al., “Crystal structures of murine and human Histamine-Releasing Factor (HRF/TCTP) and a model for HRF dimerization in mast cell activation,” Mol Immunol. 93: 216-222 (January 2018).

In some cases, a HRF multimer comprises three or more HRF monomers. In some cases, the HRF multimer comprises four or more, five or more, six or more, seven or more, eight or more, nine or more, ten or more, eleven or more, or twelve or more HRF monomers. In some cases, the HRF monomers within the HRF multimer interact with each other through non-covalent interactions (e.g., ionic interaction such as a salt-bridge interaction, hydrogen bonding, Van der Waals interaction, hydrophobic interaction, or a combination thereof), a covalent interaction (e.g., a disulfide bond), or a combination thereof. In some cases, the HRF multimer comprises two or more HRF dimers further forming a higher-ordered multimer structure, while retaining the interactions within a HRF dimer. In some cases, the HRF multimer comprises a molecular weight that is greater than 145 kD. In some cases, the HRF multimer comprises a molecular weight that is greater than 150 kD. In some cases, the HRF multimer comprises a molecular weight that is greater than 175 kD. In some cases, the HRF multimer comprises a molecular weight that is greater than 200 kD. In some cases, the HRF multimer comprises a molecular weight that is greater than 250 kD.

In some embodiments, an HRF-reactive Ig molecule comprises a HRF-reactive IgE molecule. In additional embodiments, an HRF-reactive Ig molecule comprises a HRF-reactive IgG molecule.

Additions and insertions include chimeric and fusion polypeptide or nucleic acid sequences, which is a sequence having one or more molecules not normally present in a reference native (wild type) sequence covalently attached to the sequence. The terms “fusion” or “chimeric” and grammatical variations thereof, when used in reference to a molecule, such as a HRF, means that a portions or part of the molecule contains a different entity distinct (heterologous) from the molecule (e.g., HRF, HRF fragment or antibody) as they do not typically exist together in nature. That is, for example, one portion of the fusion or chimera, such as HRF, includes or consists of a portion that does not exist together in nature, and is structurally distinct. A particular example is a molecule, such as an amino acid sequence of another protein (e.g., immunoglobulin such as an Fc domain, or antibody) attached to HRF to produce a chimera, or a chimeric polypeptide, to impart a distinct function (e.g., increased solubility, in vivo half-life, etc.). Another particular example is an amino acid sequence of another protein to produce a multifunctional protein (e.g., multifunctional HRF or multispecific antibody).

Additions and insertions also include label or a tag, which can be used to provide an agent that is detectable or that is useful for isolating the tagged entity (e.g., HRF, HRF fragment or HRF antibody). A detectable label can be attached, for example, to (e.g., linked conjugated) HRF, HRF fragment or HRF antibody, or be within or be one or more atoms that comprise the molecule.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the molecule (e.g., HRF, HRF fragment or antibody). In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Additional non-limiting examples of amino acid modifications include protein subsequences and fragments. Exemplary HRF subsequences and fragments include a portion of the HRF sequence that binds to an Ig, such as an IgE. Exemplary HRF subsequences and fragments also include an immunogenic portion of HRF.

As used herein, the term “subsequence” or “fragment” means a portion of the full length molecule. A subsequence of a polypeptide sequence, such as HRF or an antibody sequence, has one or more less amino acids than a full length HRF (e.g. one or more internal or terminal amino acid deletions from either amino or carboxy-termini). A subsequence of an antibody has one or less amino acids than a full length antibody heavy or light chain or constant region. A nucleic acid subsequence has at least one less nucleotide than a full length comparison nucleic acid sequence. Subsequences therefore can be any length up to the full length native molecule.

Functional subsequences can vary in size from a polypeptide as small as an epitope capable of binding an antibody molecule (i.e., about five amino acids) up to the entire length of a reference polypeptide. Functional HRF subsequences are at least 5-10, 10-20, 20-30, 30-40, 40-50, 50-75, 75-100, 100-150, or 150-171 amino acid residues. Functional antibody subsequences are at least 5-10, 10-20, 20-30, 30-40, 40-50, 50-75, 75-100, 100-125 amino acid residues.

Thus, in another embodiment, the disclosure provides functional polypeptides or functional subsequences thereof. In particular embodiments, a functional polypeptide or functional subsequence shares at least 50% identity with a reference sequence, for example, an HRF polypeptide sequence and that binds to an Ig, such as an IgE, or is capable of modulating HRF activity, function or expression, or an antibody that binds to the HRF sequence region that mediates HRF binding to an Ig, such as an IgE. In other embodiments, the polypeptides have at least 60%, 70%, 75% or more identity (e.g., 80%, 85% 90%, 95%, 96%, 97%, 98%, 99% or more identity) to a reference sequence, such as an HRF polypeptide sequence that binds to an Ig, such as an IgE, or is capable of modulating HRF activity, function or expression. The functional polypeptides or functional subsequences thereof including modified forms of the disclosure, such as HRF and antibodies that bind to HRF, may have one or more of the functions or biological activities described herein.

The term “identity” and grammatical variations thereof, mean that two or more referenced entities are the same. Thus, where two polypeptides (e.g., HRF or antibody) sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region.

The percent identity can extend over the entire sequence length of the polypeptide (e.g., HRF). In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology. An HRF sequence or an antibody or subsequence with substantial homology has or is predicted to have at least partial activity or function as the reference HRF sequence or antibody.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Modifications can be produced using methods known in the art (e.g., PCR based site-directed, deletion and insertion mutagenesis, chemical modification and mutagenesis, cross-linking, etc.), or may be spontaneous or naturally occurring (e.g. random mutagenesis). For example, naturally occurring allelic variants can occur by alternative RNA splicing, polymorphisms, or spontaneous mutations of a nucleic acid encoding HRF polypeptide. Further, deletion of one or more amino acids can also result in a modification of the structure of the resultant polypeptide without significantly altering a biological function or activity. Deletion of amino acids can lead to a smaller active molecule. For example, as set forth herein, removal of HRF amino or carboxy terminal or internal amino acids does not destroy Ig binding activity.

Modified HRF sequences, antibodies and subsequences fragment of the disclosure may have an affinity greater or less than 2-5, 5-10, 10-100, 100-1000 or 1000-10,000-fold affinity, or any numerical value or range within or encompassing such values, than a comparison HRF sequence or antibody. In one embodiment, an HRF sequence has a binding affinity for an Ig, such as an IgE, within about 1-5000 fold of the binding affinity of HRF amino acids 1-19 (MIIYRDLISHDEMFSDIYK (SEQ ID NO:1)) or HRF amino acids 79-142 (QETSFTKEAYKKYIKDYMKSIKGKLEEQRPERVKPFMTGAAEQIKHILANFKNYQFFIGENMNP (SEQ ID NO:2)) or an equivalent of each thereof, for binding to an Ig, such as an IgE.

The term “substantially the same” when used in reference to an HRF sequence or subsequence thereof that binds to an Ig, or an antibody or subsequence thereof that binds to HRF, means that the relative binding affinity or avidity for binding to an Ig, such as an IgE, means that the binding is within 100 fold (greater or less than) of the binding affinity of a reference HRF sequence or antibody (e.g., an Ig, such as an IgE, or HRF). Binding affinity can be determined by association (Kₐ) and dissociation (K_{D} or K_{d}) rate. Equilibrium affinity constant, K, is the ratio of Kₐ/K_{d}. Association (Kₐ) and dissociation (K_{D} or K_{d}) rates can be measured using surface plasmon resonance (SPR) (Rich and Myszka, Curr. Opin. Biotechnol. 11:54 (2000); Englebienne, Analyst. 123:1599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)). Thus, for example, if binding of a reference HRF antibody to HRF has a K_{D} of 10⁻⁹M, then an antibody which has substantially the same binding affinity as the reference antibody will have a K_{D} within the range of 10⁻⁷ M to K_{D} 10⁻¹¹ M for binding to HRF.

The disclosure also provides polynucleotides encoding HRF polypeptides and antibodies that bind to HRF. In one embodiment, a polynucleotide sequence has about 65% or more identity (e.g., 70%, 75%, 80%, 85%, 90%, 95%, 96%, 97%, 98%, 99% or more) to a sequence encoding an HRF subsequence that binds to an Ig, such as an IgE. In particular embodiments, a nucleic acid encodes all or a portion of amino acids 1-19 (MIIYRDLISHDEMFSDIYK (SEQ ID NO:1)) or amino acids 79-142 (QETSFTKEAYKKYIKDYMKSIKGKLEEQRPERVKPFMTGAAEQIKHILANFKNYQFFIGENMNP (SEQ ID NO:2)) of mammalian HRF, or an equivalent of each thereof. Such polynucleotides can therefore encode any subsequence of the HRF/TCTP sequence that includes or consists of a region of HRF that binds to an Ig, such as an IgE. Such encoded subsequences can be between 5-171 amino acid residues in length, e.g., 5-10, 10-20, 20-50, 100-150, or 150-171 amino acid residues in length.

As used herein, the terms “polynucleotide” and “nucleic acid” are used interchangeably to refer to all forms of nucleic acid, oligonucleotides, primers, and probes, including deoxyribonucleic acid (DNA) and ribonucleic acid (RNA). Polynucleotides include genomic DNA, cDNA and antisense DNA, and spliced or unspliced mRNA, rRNA tRNA and antisense RNA (e.g., RNAi). Polynucleotides include naturally occurring, synthetic, and intentionally altered or modified polynucleotides as well as analogues and derivatives. Alterations can result in increased stability due to resistance to nuclease digestion, for example. Polynucleotides can be double, single or triplex, linear or circular, and can be of any length.

Polynucleotides of the disclosure include sequences that are degenerate as a result of the genetic code. There are 20 natural amino acids, most of which are specified by more than one codon. Degenerate sequences may not selectively hybridize to other disclosure nucleic acids; however, they are nonetheless included as they encode disclosure HRF polypeptides and modified forms including subsequences thereof. Thus, in another embodiment, degenerate nucleotide sequences that encode HRF polypeptides and modified forms including subsequences thereof as set forth herein, are provided.

Polynucleotide sequences include sequences having 15-20, 20-30, 30-40, 50-50, or more contiguous nucleotides. In additional aspects, the polynucleotide sequence includes a sequence having 60 or more, 70 or more, 80 or more, 100 or more, 120 or more, 140 or more, 160 or more contiguous nucleotides, up to the full length coding sequence.

Polynucleotide sequences for HRF include complementary sequences (e.g., antisense to all or a part of HRF). Antisense polynucleotides, to decrease activity, function or expression of HRF, for example, do not require expression control elements to function in vivo. However, antisense may be encoded by a nucleic acid and such a nucleic acid may be operatively linked to an expression control element for sustained or increased expression of the encoded antisense in cells or in vivo. Sequences encoding HRF subsequences that bind to an Ig, such as an IgE also are included. Such HRF forms may decrease, reduce, inhibit, suppress, limit or control binding or interaction of the native endogenous HRF with HRF-R thereby modulating signaling.

Further included are double stranded RNA sequences from an HRF coding region. The use of double stranded RNA sequences (known as “RNAi”) for inhibiting gene expression, for example, in insects and in other organisms is known in the art (Kennerdell et al., Cell 95:1017 (1998); Fire et al., Nature, 391:806 (1998)).

Such complementary, antisense and RNAi sequences can interfere with HRF activity, function or expression and be useful for modulating HRF. An effective amount of complementary, antisense or RNAi sequences from the coding region of HRF can inhibit HRF activity, function or expression and are therefore useful in the therapeutic and other methods of treatment as described herein. Such disclosure polynucleotides can be further contained within carriers or vectors suitable for passing through a cell membrane for cytoplasmic delivery, and can be modified so as to be nuclease resistant in order to enhance their stability or efficacy in the disclosure methods and compositions, for example.

Thus, in another embodiment, polynucleotides encoding HRF including the nucleotide sequence encoding full length or a subsequence of: MIIYRDLISHDEMFSDIYKIREIADGLCLEVEGKMVSRTEGNIDDSLIGGNASAEGPEGEGTE STVITGVDIVMNHHILQETSFTKEAYKKYIKDYMKSIKGKLEEQRPERVKPFMTGAAEQIKH ILANFKNYQFFIGENMNPDGMVALLDYREDGVTPYMIFFKDGLEMEKC, (SEQ ID NO:3) or an equivalent thereof, as well as nucleic acid sequences complementary to the sequence or subsequence (e.g., complementary, antisense polynucleotides), are provided.

Polynucleotides encoding full length or subsequences of HRF polypeptide are included herein. Particular examples are nucleic acid sequences that encode HRF functional subsequences. As used herein, the term “functional polynucleotide” denotes a polynucleotide that encodes a functional polypeptide as described herein. Thus, the disclosure includes polynucleotides encoding a polypeptide having a function or activity of an amino acid sequence set forth in HRF, e.g., MIIYRDLISHDEMFSDIYKIREIADGLCLEVEGKMVSRTEGNIDDSLIGGNASAEGPEGEGTE STVITGVDIVMNHHLQETSFTKEAYKKYIKDYMKSIKGKLEEQRPERVKPFMTGAAEQIKH ILANFKNYQFFIGENMNPDGMVALLDYREDGVTPYMIFFKDGLEMEKC (SEQ ID NO: 3), or an equivalent thereof.

Additional polynucleotides include fragments of the above-described nucleic acid sequences that are at least 15 bases in length, which is of sufficient length to permit a selective hybridization to an HRF nucleic acid. Polynucleotide fragments of at least 15 bases in length can be used to screen for HRF related genes in other organisms, such as mammals or insects, and are referred to herein as “probes.”

Disclosure probes and agents additionally can have a “tag” or “label” or “detectable moiety” linked thereto that provides a means of isolation or identification, or a detection signal (e.g., radionuclides, fluorescent, chemi- or other luminescent moieties). If necessary, additional reagents can be used in combination with the detectable moieties to provide or enhance the detection signal. Such labels and detectable moieties also can be linked to disclosure HRF polypeptides, nucleic acids, antibodies, and modified forms disclosed herein.

Thus, in accordance with the disclosure there are provided isolated polynucleotides that selectively hybridize to the polynucleotides described herein. In one embodiment, an isolated polynucleotide sequence hybridizes under stringent conditions to a polynucleotide encoding full length or a subsequence of HRF, e.g., encoding all or a subsequence of: MIIYRDLISHDEMFSDIYKIREIADGLCLEVEGKMVSRTEGNIDDSLIGGNASAEGPEGEGTE STVITGVDIVMNHHLQETSFTKEAYKKYIKDYMKSIKGKLEEQRPERVKPFMTGAAEQIKH ILANFKNYQFFIGENMNPDGMVALLDYREDGVTPYMIFFKDGLEMEKC (SEQ ID NO:3) or its complement or an equivalent of each thereof. In another embodiment, an isolated polynucleotide sequence hybridizes under stringent conditions to a polynucleotide encoding full length or a subsequence of HRF sequence set forth herein or its complement.

Hybridization refers to binding between complementary nucleic acid sequences (e.g., sense/antisense). As used herein, the term “selective hybridization” refers to hybridization under moderately stringent or highly stringent conditions, which can distinguish HRF related nucleotide sequences from unrelated sequences. Screening procedures which rely on hybridization allow isolation of related nucleic acid sequences, from any organism.

In nucleic acid hybridization reactions, conditions used in order to achieve a particular level of stringency will vary, depending on the nature of the nucleic acids being hybridized. For example, the length, degree of sequence complementarity, sequence composition (e.g., the GC v. AT content), and type (e.g., RNA v. DNA) of the hybridizing regions can be considered in selecting particular hybridization conditions. An additional consideration is whether one of the nucleic acids is immobilized, for example, on a filter. As is understood by those skilled in the art, the Tm (melting temperature) refers to the temperature at which the binding between two sequences is no longer stable. For two sequences to form a stable hybrid, the temperature of the reaction must be less than the Tm for the particular hybridization conditions. In general, the stability of a nucleic acid hybrid decreases as the sodium ion decreases and the temperature of the hybridization reaction increases.

Typically, wash conditions are adjusted so as to attain the desired degree of stringency. Thus, hybridization stringency can be determined, for example, by washing at a particular condition, e.g., at low stringency conditions or high stringency conditions, or by using each of the conditions, e.g., for 10-15 minutes each, in the order listed below, repeating any or all of the steps listed. Optimal conditions for selective hybridization will vary depending on the particular hybridization reaction involved.

A moderately stringent hybridization refers to a condition in which hybridization of an oligonucleotide to a target sequence comprises one or more mismatches. An example of a moderately stringent hybridization condition is as follows: 2×SSC/0.1% SDS at about 37° C. or 42° C. (hybridization conditions); 0.5×SSC/0.1% SDS at about room temperature (low stringency wash); 0.5×SSC/0.1% SDS at about 42° C. (moderate stringency wash). An example of a moderately-high stringent hybridization condition is as follows: 2×SSC/0.1% SDS at about 37° C. or 42° C. (hybridization conditions); 0.5×SSC/0.1% SDS at about room temperature (low stringency wash); 0.5×SSC/0.1% SDS at about 42° C. (moderate stringency wash); and 0.1×SSC/0.1% SDS at about 52° C. (moderately-high stringency wash). A high stringency hybridization refers to a condition in which hybridization of an oligonucleotide to a target sequence comprises no mismatches (or perfect complementarity). An example of high stringency hybridization conditions is as follows: 2×SSC/0.1% SDS at about room temperature (hybridization conditions); 0.5×SSC/0.1% SDS at about room temperature (low stringency wash); 0.5×SSC/0.1% SDS at about 42° C. (moderate stringency wash); and 0.1×SSC/0.1% SDS at about 65° C. (high stringency wash).

Polynucleotides of the disclosure can be obtained using various standard cloning and chemical synthesis techniques. Purity of polynucleotides can be determined through sequencing, gel electrophoresis and the like. For example, nucleic acids can be isolated using hybridization as set forth herein or computer-based database screening techniques known in the art. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

The disclosure HRF polynucleotides can include an expression control element distinct from the endogenous HRF gene (e.g., a non-native element), or exclude a control element from the native HRF gene to control expression of an operatively linked HRF nucleic acid. Such polynucleotides containing an expression control element controlling expression of a nucleic acid can be modified or altered as set forth herein, so long as the modified or altered polynucleotide has one or more functions or activities.

For expression in cells, disclosure polynucleotides, if desired, may be inserted into a vector. Accordingly, disclosure compositions and methods further include polynucleotide sequences inserted into a vector. The term “vector” refers to a plasmid, virus or other vehicle known in the art that can be manipulated by insertion or incorporation of a polynucleotide. Such vectors can be used for genetic manipulation (i.e., “cloning vectors”) or can be used to transcribe or translate the inserted polynucleotide (i.e., “expression vectors”). A vector generally contains at least an origin of replication for propagation in a cell and a promoter. Control elements, including expression control elements as set forth herein, present within a vector are included to facilitate proper transcription and translation (e.g., splicing signal for introns, maintenance of the correct reading frame of the gene to permit in-frame translation of mRNA and, stop codons etc.).

Compositions and methods of the disclosure are applicable to treating numerous disorders. Disorders treatable in accordance with the disclosure include disorders in which decreasing, reducing, inhibiting, suppressing, limiting or controlling a response mediated or associated with HRF activity, function or expression can provide a subject with a benefit. Disorders include undesirable or aberrant immune responses, immune disorders and immune diseases including, for example, food allergy, allergic reaction, hypersensitivity, inflammatory response, inflammation, and airway constriction.

As used herein, an “undesirable immune response” or “aberrant immune response” refers to any immune response, activity or function that is greater or less than desired or physiologically normal. An undesirable immune response, function or activity can be a normal response, function or activity. Thus, normal immune responses so long as they are undesirable, even if not considered aberrant, are included within the meaning of these terms. An undesirable immune response, function or activity can also be an abnormal response, function or activity. An abnormal (aberrant) immune response, function or activity deviates from normal. Undesirable and aberrant immune responses can be humoral, cell-mediated or a combination thereof, either chronic or acute.

One non-limiting example of an undesirable or aberrant immune response is where the immune response is hyper-responsive, such as in the case of an autoimmune disorder or disease. Another example of an undesirable or aberrant immune response is where an immune response leads to acute or chronic inflammatory response or inflammation in any tissue or organ, such as an allergy (e.g., food allergy or asthma).

The terms “immune disorder” and “immune disease” mean, an immune function or activity, that is greater than (e.g., autoimmunity) or less than (e.g., immunodeficiency) desired, and which is characterized by different physiological symptoms or abnormalities, depending upon the disorder or disease. Particular non-limiting examples of immune disorders and diseases to which the disclosure applies include, for example, food allergy, allergic reaction, hypersensitivity, inflammatory response, inflammation, and airway constriction. Additional disorders are generally characterized as an undesirable or aberrant increased or inappropriate response, activity or function of the immune system. Disorders and diseases that can be treated in accordance with the disclosure include, but are not limited to, disorders and disease that cause cell or tissue/organ damage in the subject.

In accordance with the disclosure, there are provided methods of treating a food allergy. In one embodiment, a method includes contacting histamine releasing factor (HRF)/translationally controlled tumor protein (TCTP) with a compound that inhibits or reduces binding of HRF/TCTP to an immunoglobulin thereby treating the food allergy.

As used herein, the term “food allergy” refers to an adverse immune response to food proteins, which is unlike lactose intolerance, food poisoning or food aversions. A particular type of food allergy is a “rapid” type of food allergy, often called as “IgE-mediated” food hypersensitivity. Symptoms of food allergy may include systemic anaphylaxis (e.g., hypotension, loss of consciousness, and death), skin (e.g., flushing, urticaria, angioedema, and worsening eczema), eye (e.g., allergic conjunctivitis), gut (e.g., nausea, cramping, vomiting, diarrhea, and abdominal pain), and respiratory tract reactions (e.g., rhinitis and asthma) (Sicherer et al., Pediatrics 102:e6 (1998); Atkins et al., J. Allergy Clin. Immunol. 75:356 (1985); Lack, N. Engl. J. Med. 359:1252 (2008)). Examples of known food allergens include eggs, peanuts, tree nuts, fish, and shellfish are common allergens in both children and adults, while children also often react to wheat, and soy.

Food-induced allergic reactions result from immunologic pathways that include activation of effector cells through food specific IgE antibodies, cell-mediated (non-IgE-mediated) reactions resulting in subacute or chronic inflammation, or the combination of these pathways. The significance of IgE-mediated arm of reactions in human was demonstrated by anti-IgE therapy in patients with peanut allergy, which significantly and substantially increased the threshold of sensitivity to peanut on oral food challenge (Leung et al., N. Engl. J. Med. 348:986 (2003)). Furthermore, histamine has been reported to increase in food allergy patients after allergen challenge (Sampson and Jolie, N. Engl. J. Med. 311:372 (1984)), suggesting involvement of mast cell or basophil activation downstream of IgE-mediated pathways. On the other hand, celiac disease, which is a representative of the cell-mediated arm of food hypersensitivity, is mediated by gluten-reactive T cells, and the symptoms are confined to gut, often mild and chronic (Sollid and Lundin, Mucosal Immunol. 2:3 (2009)).

Consequently, methods of the disclosure include modulating (e.g., decrease, reduce, inhibit, suppress, limit or control) immunologic pathways that include activation of effector cells through food specific IgE antibodies, cell-mediated (non-IgE-mediated) reactions resulting in subacute or chronic inflammation, or a combination of these pathways. Methods of the disclosure also include modulating (e.g., decrease, reduce, inhibit, suppress, limit or control) IgE-mediated reactions thereby treating, inhibiting, reducing or decreasing sensitivity to food allergies. Methods of the disclosure further include modulating (e.g., decrease, reduce, inhibit, suppress, limit or control) histamine release or activation of downstream IgE-mediated pathways, such as by, but not limited to, mast cells or basophils.

As disclosed herein, animal models indicate that food allergy may be elicited by classic or alternative pathways, both of which appear to depend on immunoglobulins (Igs). Methods of the disclosure therefore also include modulating (e.g., decrease, reduce, inhibit, suppress, limit or control) either or both pathways that appear to contribute to food allergy. Such methods include, for example, modulating (e.g., decrease, reduce, inhibit, suppress, limit or control) B cells or mast cells, or release of histamine and platelet activating factor (PAF), which is believed to cause anaphylactic symptoms. Such methods also include, for example, modulating (e.g., decrease, reduce, inhibit, suppress, limit or control) hypothermia.

As also disclosed herein, animal models indicate that the Ig/mast cell axis plays a role in food allergy. The food-induced anaphylaxis and diarrhea models reveal the role of B cells and mast cells, and a contributory role of FcεRI, suggesting a central role of Ig/mast cell interaction in the effecter phase of food allergy. As disclosed herein, a subset of IgE and IgG molecules have been identified as HRF receptors, and the HRF reactivity of Igs could modulate the effecter phase of food allergy.

Methods of the disclosure therefore also include modulating numbers or activity of mast cells or B cells in the small intestine, such as in the jejunum, or in the colon (large intestine). Such methods include, for example, decreasing, reducing, inhibiting, suppressing, limiting or controlling numbers or activity of mast cells or B cells in the small intestine, such as in the jejunum. Such methods also include, for example, inhibiting, reducing or decreasing numbers or activity of mast cells or B cells in the colon (large intestine). Such methods further include treating diarrhea.

As further disclosed herein, food allergy is prevalent in patients with eosinophilic esophagitis (EoE), and HRF appears to play a role in food allergy or the underlying eosinophilic esophagitis (EoE). HRF therefore plays a role in food allergy and EoE.

Methods of the disclosure are therefore applicable to treatment of food allergy in subjects with eosinophilic esophagitis (EoE). Methods of the disclosure therefore include treatment (e.g., decrease, reduce, inhibit, suppress, limit or control) of one or more symptoms, such as vomiting, abdominal pain, or failure to thrive, for example, in young children, or, for example, dysphagia in adolescents or adults. Methods of the disclosure also include treatment (e.g., decrease, reduce, inhibit, suppress, limit or control) of esophageal stricture formation and tissue remodeling. Methods of the disclosure further include treatment of (e.g., decrease, reduce, inhibit, suppress, limit or control) esophageal dysmotility in a subject, such as in an adult or pediatric EoE subject. Methods of the disclosure additionally include treatment of (e.g., decrease, reduce, inhibit, suppress, limit or control) local IgE production and systemic sensitization that occur in EoE. Methods of the disclosure moreover include treatment (e.g., decrease, reduce, inhibit, suppress, limit or control) of delayed type hypersensitivity and esophageal mastocytosis, such as in patients suffering from EoE (Kirsh et al., J. Pediatric Gastroenterol. Nutrition 44:20 (2007)).

The term “contacting” means direct or indirect binding or interaction between two or more entities (e.g., between an HRF sequence and native endogenous HRF, or between an antibody and endogenous HRF). A particular example of direct interaction is binding. A particular example of an indirect interaction is where one entity acts upon an intermediary molecule, which in turn acts upon the second referenced entity. Thus, for example, contacting HRF with an antibody includes allowing the antibody to bind to HRF, or allowing the antibody to act upon an intermediary that in turn binds to HRF. Contacting as used herein includes in solution, in solid phase, in vitro, ex vivo, in a cell and in vivo. Contacting in vivo can be referred to as administering, or administration.

In accordance with the disclosure, there are provided methods of treating an allergic reaction, hypersensitivity, an inflammatory response or inflammation. In one embodiment, a method includes contacting histamine releasing factor (HRF)/translationally controlled tumor protein (TCTP) with a compound that inhibits or reduces binding of HRF/TCTP to an immunoglobulin thereby treating the allergic reaction, hypersensitivity, inflammatory response or inflammation.

In accordance with the disclosure, there are also provided methods for decreasing, reducing, inhibiting, suppressing, limiting or controlling the probability, severity, frequency, duration or preventing a subject from having an acute or chronic food allergy, allergic reaction, hypersensitivity, an inflammatory response or inflammation. In one embodiment, a method includes administering to a subject a compound that decreases, reduces, inhibits, suppresses, limits or controls binding of HRF/TCTP to an immunoglobulin thereby decreasing, reducing, inhibiting, suppressing, limiting or controlling the probability, severity, frequency, duration or preventing the subject from having an acute or chronic food allergy, allergic reaction, hypersensitivity, inflammatory response or inflammation.

Methods of the disclosure include modulating (e.g., decrease, reduce, inhibit, suppress, limit or control) one or more functions, activities or expression of HRF, in vitro, ex vivo or in vivo. As used herein, the term “modulate,” means an alteration or effect of the term modified. For example, the term modulate can be used in various contexts to refer to an alteration or effect of an activity, a function, or expression of a polypeptide, gene or signaling pathway, or a physiological condition or response of an organism. Thus, where the term “modulate” is used to modify the term “HRF” this means that an HRF activity, function, or expression is altered or affected (e.g., decreased, reduced, inhibited, suppressed, limited, controlled or prevented, etc.) Detecting an alteration or an effect on HRF activity, function or expression can be determined as set forth herein using in vitro assays or an animal model.

Compounds useful in practicing the methods of the disclosure include peptides and polypeptides, such as HRF sequences, HRF subsequences or fragments (e.g., a sequence that binds to an Ig, such as an IgE), antibodies and antibody subsequences (e.g., polyclonal or monoclonal and any of IgM, IgG, IgA, IgD or IgE isotypes) known to the skilled artisan and as set forth herein. Such sequences can be mammalian, humanized, human or chimeric.

Particular examples include a fragment of HRF/TCTP polypeptide that binds to an immunoglobulin, such as an IgE. An exemplary HRF sequence includes or consists of amino acids 1-19 or amino acids 79-142 of a mammalian HRH/TCTP sequence, for example, all or a portion of a MIIYRDLISHDEMFSDIYK (SEQ ID NO:1) sequence, or all or a portion of a QETSFTKEAYKKYIKDYMKSIKGKLEEQRPERVKPFMTGAAEQIKHILANFKNYQFFIGENMNP (SEQ ID NO:2) sequence, or an equivalent of each thereof.

Particular non-limiting examples of HRF binding antibodies include commercial antibodies from Santa Cruz Biotechnology, Inc. (Santa Cruz, Calif.), as set forth in Table 2 below:

TABLE 2

| | | |
|---|---|---|
| HRF (B-3) Antibody sc-133131 | mouse IgG₁ | 1-172 (h) |
| HRF (FL-172) Antibody sc-30124 | rabbit IgG | FL (h) |
| HRF (L-20) Antibody sc-20427 | goat IgG | internal (h) |
| HRF (N-20) Antibody sc-20426 | goat IgG | N-terminus (h) |
| HRF (23-Y) Antibody sc-100763 | mouse IgG₁ | FL (h) |
| HRF (20) Antibody sc-135940 | mouse IgG₁ | N/A |

Additional particular non-limiting examples of HRF binding antibodies include commercial antibodies from Assay Designs/Stressgen (Ann Arbor, Mich.), Proteintech Group (Chicago, Ill.), R and D Systems, Inc. (Minneapolis, Minn.), Sigma-Aldrich Corp. (St. Louis, Mo.), AbDSerotec/MorphoSys UK Ltd. (Oxford, UK), Strategic Diagnostics (SDIX) (Newark, Del.), Abcam (Cambridge, Mass.) and Novus Biologicals, LLC (Littleton, Colo.) as set forth in Table 3 below:

TABLE 3
Tumor protein (TPT1) Monoclonal Antibody (3C7), Assay Designs/
Stressgen, reactivity: human; clonality: monoclonal; host: mouse
TPT1, Proteintech Group, reactivity: human; clonality: polyclonal;
host: rabbit
Human/Mouse/Rat TPT1/TCTP MAb (Clone 488411), R& D
Systems, reactivity: human; clonality: monoclonal; host: mouse
Monoclonal Anti-TPT1 antibody produced in mouse, Sigma-
Aldrich, reactivity: human; clonality: monoclonal; host: mouse
Mouse anti-human TPT1: Azide Free, AbDSerotec, reactivity:
human; clonality: monoclonal; host: mouse
TPT1 antibody, Strategic Diagnostics, reactivity: human; clonality:
polyclonal; host: rabbit
TCTP antibody, Abcam, reactivity: human, rat, mouse; clonality:
polyclonal; host: rabbit
TCTP antibody, Abcam, reactivity: human; clonality: monoclonal;
host: mouse
TCTP antibody, Abcam, reactivity: human; clonality: monoclonal;
host: mouse
TCTP Antibody, Novus Biologicals, reactivity: human, rat; clonality:
polyclonal; host: rabbit
TCTP Antibody (3C7), Novus Biologicals, reactivity: human; clonality:
monoclonal; host: mouse
TCTP Antibody (2C4), Novus Biologicals, reactivity: human; clonality:
monoclonal; host: mouse

The condition treated in accordance with the methods can be chronic or acute. For example, food allergy, allergic reaction, hypersensitivity, inflammatory response, inflammation, or airway constriction can be chronic or acute.

Methods (e.g., treatment) according to the disclosure can result in a reduction in occurrence, frequency, severity, progression, or duration of a symptom of the condition. For example, methods of the disclosure can protect against or decrease, reduce, inhibit, suppress, limit or control progression, severity, frequency, duration or probability of an adverse symptom of the food allergy, allergic reaction, hypersensitivity, inflammatory response or inflammation.

In particular embodiments, treatment according to a method of the disclosure is sufficient to protect against or decrease, reduce, inhibit, suppress, limit or control the food allergy, allergic reaction, hypersensitivity, inflammatory response or inflammation, decrease, reduce, inhibit, suppress, limit or control susceptibility to the food allergy, allergic reaction or hypersensitivity, or decrease, reduce, inhibit, suppress, limit or control a food allergy, allergic reaction, hypersensitivity, inflammatory response or inflammation.

In additional particular embodiments, treatment according to a method of the disclosure is sufficient to decrease, reduce, inhibit, suppress, limit, control or improve the probability, severity, frequency, or duration of one or more adverse symptoms, disorders, illnesses, pathologies, diseases, or complications caused by or associated with the food allergy, allergic reaction, hypersensitivity, inflammatory response or inflammation.

Exemplary symptoms include one or more of diarrhea, bloat, swelling, pain, rash, headache, fever, nausea, lethargy, airway constriction, skeletal joint stiffness, or tissue or cell damage. Exemplary symptoms also include tissue, organ or cellular damage or remodeling. Exemplary tissues and organs that can exhibit damage include epidermal or mucosal tissue, gut, bowel, pancreas, thymus, liver, kidney, spleen, skin, eye, or a skeletal joint (e.g., knee, ankle, hip, shoulder, wrist, finger, toe, or elbow), and airway. Treatment can result in decreasing, reducing, inhibiting, suppressing, limiting, controlling or preventing progression or worsening of tissue, organ or cellular damage or remodeling.

Methods of the disclosure that include treatment include protecting against or decreasing, reducing, inhibiting, suppressing, limiting or controlling occurrence, progression, severity, frequency or duration of a symptom or characteristic of the condition treated. At the whole body, regional or local level, a symptom of an inflammatory response or inflammation is generally characterized by swelling, pain, headache, fever, nausea, skeletal joint stiffness or lack of mobility, rash, redness or other discoloration. At the whole body level, an adverse symptom can also include shortness of breath (dyspnea), wheezing, stridor, coughing, airway remodeling, rapid breathing (tachypnea), prolonged expiration, runny nose, rapid or increased heart rate (tachycardia), rhonchous lung, lung or airway constriction, over-inflation of the chest or chest-tightness, decreased lung capacity, an acute asthmatic episode, lung, airway or respiratory mucosum inflammation, or lung, airway or respiratory mucosum tissue damage.

At the cellular level, a symptom of an inflammatory response or inflammation is characterized by one or more of cell infiltration of the region, production of antibodies, production of cytokines, lymphokines, chemokines, interferons and interleukins, cell growth and maturation factors (e.g., differentiation factors), cell proliferation, cell differentiation, cell accumulation or migration and cell, tissue or organ damage or remodeling. Thus, treatment according to a method of the disclosure can protect against or decrease, reduce, inhibit, suppress, limit or control occurrence, progression, severity, frequency or duration of any one or more of such symptoms or characteristics of the condition.

Allergic reactions in which treatment according to a method of the disclosure can protect against or decrease, reduce, inhibit, suppress, limit or control include bronchial asthma (extrinsic or intrinsic); Allergic rhinitis; Onchocercal dermatitis; Atopic dermatitis; allergic conjunctivitis; Drug reactions; Nodules, eosinophilia, rheumatism, dermatitis, and swelling (NERDS); Esophageal and a gastrointestinal allergy (e.g., a food allergy).

Conditions in which treatment according to a method of the disclosure can protect against or decrease, reduce, inhibit, suppress, limit or control include hypersensitivity, inflammatory response or inflammation of a respiratory disease or disorder. Such disorders can affect the skin, or upper or lower respiratory tract, and include, for example, asthma, allergic asthma, bronchiolitis and pleuritis, as well as Airway Obstruction, Apnea, Asbestosis, Atelectasis, Berylliosis, Bronchiectasis, Bronchiolitis, Bronchiolitis Obliterans Organizing Pneumonia, Bronchitis, Bronchopulmonary Dysplasia, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Rhinoscleroma, Scimitar Syndrome, Severe Acute Respiratory Syndrome, Silicosis, Tracheal Stenosis, eosinophilic pleural effusions, Histiocytosis; chronic eosinophilic pneumonia; hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease (COPD); Interstitial Lung Disease; and Topical eosinophilia.

In some instances, exemplary allergic asthma subtypes include, but are not limited to, an IgE-mediated allergic response, an allergen-driven Th2-mediated inflammation, and allergic rhinitis. Exemplary allergens include, but are not limited to, environmental allergens such as polluants, dust, pollen, dust mite, pet hair, or mold; and pathogens such as viruses (e.g., Rhinovirus) and bacteria (e.g., Staphylococcus aureus). In some cases, an allergic asthma subtype is rhinovirus-induced asthma exacerbation. In some cases, an allergic asthma is as described in Froidure, et al., “Asthma phenotypes and IgE responses,” Eur Respir J 47: 304-319, 2016.

In accordance with the disclosure, there are also provided methods for increasing, enhancing or stimulating airway-dilation, and for reducing or inhibiting airway-constriction. In one embodiment, a method includes administering to a subject in need of increasing airway-dilation an amount of a compound that inhibits or reduces binding of HRF/TCTP to an immunoglobulin sufficient to increase, enhance or stimulate airway-dilation in the subject. In another embodiment, a method includes administering to a subject in need thereof an amount of a compound that inhibits or reduces binding of HRF/TCTP to an immunoglobulin sufficient to reduce or inhibit airway-constriction in the subject.

In methods of the disclosure, a compound can be administered prior to, substantially contemporaneously with or following one or more adverse symptoms, disorders, illnesses, pathologies, diseases, or complications caused by or associated with development of or manifestation of an acute or chronic symptom, for example, a food allergy, allergic reaction, hypersensitivity, inflammatory response or inflammation. In methods of the disclosure, a compound can be administered prior to, substantially contemporaneously with or following administering a second drug or treatment.

Non-limiting examples of classes of second drugs or treatments include an anti-food allergy, anti-allergic reaction, anti-hypersensitivity, anti-inflammatory, anti-asthmatic or anti-allergy drug. More particular examples of a second drug include a hormone, a steroid, an anti-histamine, anti-leukotriene, anti-IgE, anti-α4 integrin, anti-β2 integrin, anti-CCR3 antagonist, β2 agonist or an anti-selectin.

Methods of the disclosure may be practiced prior to (i.e. prophylaxis), concurrently with or after evidence of the disorder, disease or condition beginning (e.g., one or more symptoms). Administering a composition prior to, concurrently with or immediately following development of a symptom may decrease, reduce, inhibit, suppress, limit or control the occurrence, frequency, severity, progression, or duration of one or more adverse symptoms, disorders, illnesses, pathologies, diseases, or complications caused by or associated with the food allergy, allergic reaction, hypersensitivity, inflammatory response or inflammation in the subject. In addition, administering a composition prior to, concurrently with or immediately following development of one or more symptoms may decrease, reduce, inhibit, suppress, limit, control or prevent damage to cells, tissues or organs that occurs, for example, due to one or more adverse symptoms, disorders, illnesses, pathologies, diseases, or complications caused by or associated with a food allergy, allergic reaction, hypersensitivity, inflammatory response or inflammation.

Compositions and the methods of the disclosure, such as treatment methods, can provide a detectable or measurable therapeutic benefit or improvement to a subject. A therapeutic benefit or improvement is any measurable or detectable, objective or subjective, transient, temporary, or longer-term benefit to the subject or improvement in the condition, disorder or disease, or one or more adverse symptoms, disorders, illnesses, pathologies, diseases, or complications caused by or associated with the food allergy, allergic reaction, hypersensitivity, inflammatory response or inflammation. Therapeutic benefits and improvements include, but are not limited to, decreasing, reducing, inhibiting, suppressing, limiting or controlling the occurrence, frequency, severity, progression, or duration of one or more symptoms or complications associated with disorders, illnesses, pathologies, diseases, or complications caused by or associated with the food allergy, allergic reaction, hypersensitivity, inflammatory response or inflammation. Compositions and methods of the disclosure therefore include providing a therapeutic benefit or improvement to a subject.

In the methods of the disclosure in which a therapeutic benefit or improvement is a desired outcome, a composition of the disclosure such as an HRF polypeptide or an antibody that binds to HRF, can be administered in a sufficient or effective amount to a subject in need thereof. An “amount sufficient” or “amount effective” refers to an amount that provides, in single or multiple doses, alone or in combination, with one or more other compositions (therapeutic agents such as a drug), treatments, protocols, or therapeutic regimens agents, a detectable response of any duration of time (long or short term), an expected desired outcome in or a benefit to a subject of any measurable or detectable degree or for any duration of time (e.g., for minutes, hours, days, months, years, or cured). For example, a sufficient amount of an HRF sequence, or an antibody or subsequence that binds to HRF, is considered as having a therapeutic effect if administration results in a decreased or reduced amount or frequency of immunotherapy being required for treatment of a one or more adverse symptoms, disorders, illnesses, pathologies, diseases, or complications caused by or associated with the food allergy, allergic reaction, hypersensitivity, inflammatory response or inflammation.

The doses or “sufficient amount” or “effective amount” for treatment (e.g., to provide a therapeutic benefit or improvement) typically are effective to ameliorate a disorder, disease or condition, or one, multiple or all adverse symptoms, consequences or complications of the disorder, disease or condition, one or more adverse symptoms, disorders, illnesses, pathologies, diseases, or complications, for example, caused by or associated with the food allergy, allergic reaction, hypersensitivity, inflammatory response or inflammation to a measurable extent, although decreasing, reducing, inhibiting, suppressing, limiting or controlling a progression or worsening of the disorder, disease or condition or a symptom, is a satisfactory outcome.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

An amount sufficient or an amount effective can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, status of the disorder, disease or condition treated or the side effects of treatment. In addition, an amount sufficient or an amount effective need not be sufficient or effective if given in single or multiple doses without a second composition (e.g., agent), treatment, protocol or therapeutic regimen, since additional doses, amounts or duration above and beyond such doses, or additional compositions (e.g., agents), treatments, protocols or therapeutic regimens may be included in order to be considered effective or sufficient in a given subject. Amounts considered sufficient also include amounts that result in a reduction of the use of another treatment, therapeutic regimen or protocol.

An amount sufficient or an amount effective need not be effective in each and every subject treated, prophylactically or therapeutically, nor a majority of treated subjects in a given group or population. An amount sufficient or an amount effective means sufficiency or effectiveness in a particular subject, not a group or the general population. As is typical for such methods, some subjects will exhibit a greater response, or less or no response to a treatment method.

Additional examples of a therapeutic benefit for one or more adverse symptoms, disorders, illnesses, pathologies, diseases, or complications caused by or associated with the food allergy, allergic reaction, hypersensitivity, inflammatory response or inflammation is an improvement in one or more such symptoms. For example, decreasing, reducing, inhibiting, suppressing, limiting, controlling or preventing further or reducing an allergic reaction, food allergy, lung or airway constriction or remodeling, tissue or organ infiltration or tissue destruction, or pancreas, thymus, kidney, liver, spleen, eye, epidermal (skin) or mucosal tissue, gut or bowel infiltration or tissue destruction or remodeling.

Particular non-limiting examples of therapeutic benefit or improvement for a pathogen include decreasing, reducing, inhibiting, suppressing, limiting, controlling or preventing occurrence, frequency, severity, progression, or duration of one or more symptoms or complications of a food allergy, allergic reaction, hypersensitivity, inflammatory response or inflammation. Additional particular non-limiting examples of therapeutic benefit or improvement include stabilizing the condition (i.e., decreasing, reducing, inhibiting, suppressing, limiting, controlling or preventing a worsening or progression of a symptom or complication associated with a food allergy, allergic reaction, hypersensitivity, inflammatory response or inflammation). Symptoms or complications associated with a food allergy, allergic reaction, hypersensitivity, inflammatory response or inflammation whose occurrence, frequency, severity, progression, or duration can be decreased, reduced, inhibited, suppressed, limited, controlled or prevented are known in the art. A therapeutic benefit can also include reducing susceptibility of a subject to one or more adverse symptoms, disorders, illnesses, pathologies, diseases, or complications caused by or associated with a food allergy, allergic reaction, hypersensitivity, inflammatory response or inflammation or hastening or accelerating recovery from one or more adverse symptoms, disorders, illnesses, pathologies, diseases, or complications caused by or associated with a food allergy, allergic reaction, hypersensitivity, inflammatory response or inflammation.

As is typical for treatment or therapeutic methods, some subjects will exhibit greater or less response to a given treatment, therapeutic regiment or protocol. Thus, appropriate amounts will depend upon the condition treated (e.g., the type or stage of the tumor), the therapeutic effect desired, as well as the individual subject (e.g., the bioavailability within the subject, gender, age, etc.).

The term “subject” refers to animals, typically mammalian animals, such as humans, non-human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of food allergy, allergic reaction, hypersensitivity, inflammatory response or inflammation, for studying in vivo a composition of the disclosure, for example, an HRF sequence or an antibody that binds to HRF.

Subjects appropriate for treatment include those having or at risk of having an undesirable or aberrant immune response, immune disorder or immune disease, those undergoing treatment for an undesirable or aberrant immune response, immune disorder or immune disease as well as those who are undergoing or have undergone treatment or therapy for an undesirable or aberrant immune response, immune disorder or immune disease, including subjects where the undesirable or aberrant immune response, immune disorder or immune disease is in remission. Specific non-limiting examples include subjects having or at risk of having one or more adverse symptoms, disorders, illnesses, pathologies, diseases, or complications caused by or associated with a food allergy, allergic reaction, hypersensitivity, inflammatory response or inflammation.

“At risk” subjects typically have risk factors associated with undesirable or aberrant immune response, immune disorder or immune disease, such as a food allergy, allergic reaction, hypersensitivity, inflammatory response or inflammation. Risk factors include gender, lifestyle (diet, smoking), occupation (medical and clinical personnel, agricultural and livestock workers), environmental factors (allergen exposure), family history (e.g., genetic predisposition), etc.

Compositions and methods of the disclosure may be contacted or provided in vitro, ex vivo or administered in vivo. Compositions can be administered to provide the intended effect as a single or multiple dosages, for example, in an effective or sufficient amount. Exemplary doses range from about 25-250, 250-500, 500-1000, 1000-2500 or 2500-5000, 5000-25,000, 5000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 5000-50,000 mg/kg, on consecutive days, alternating days or intermittently.

Single or multiple doses can be administered on the same or consecutive days, alternating days or intermittently. For example, a compound such as an HRF sequence or antibody that binds to HRF can be administered one, two, three, four or more times daily, on alternating days, bi-weekly, weekly, monthly, bi-monthly, or annually.

Compounds can be administered to a subject and methods may be practiced substantially contemporaneously with, or within about 1-60 minutes, hours, or days of the onset of an adverse symptom associated with a food allergy, allergic reaction, hypersensitivity, inflammatory response or inflammation.

Compounds can be administered and methods may be practiced via systemic, regional or local administration, by any route. For example, an HRF sequence or an antibody that binds to HRF may be administered systemically, regionally or locally, via ingestion, via inhalation, topically, intravenously, orally (e.g., ingestion or inhalation), intramuscularly, intraperitoneally, intradermally, subcutaneously, intracavity, intracranially, transdermally (topical), parenterally, e.g. transmucosally or rectally. Compositions and methods of the disclosure including pharmaceutical formulations can be administered via a (micro) encapsulated delivery system or packaged into an implant for administration.

Disclosure compositions and methods include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations can be contained in a tablet (coated or uncoated), capsule (hard or soft), microbead, emulsion, powder, granule, crystal, suspension, syrup or elixir.

Pharmaceutical compositions can be formulated to be compatible with a particular route of administration. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin, can prolong absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the disclosure (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The compositions used in accordance with the disclosure, including proteins (HRF sequences, antibodies), nucleic acid (e.g., inhibitory), treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The disclosure provides cell-free (e.g., in solution, in solid phase) and cell-based (e.g., in vitro or in vivo) methods of screening, detecting and identifying HRF. The methods can be performed in solution, in vitro using a biological material or sample, and in vivo, for example, using a fluid or lavage sample from an animal.

In accordance with the disclosure, there are provided methods of diagnosing a subject having or at risk of a food allergy. In one embodiment, a method includes measuring histamine releasing factor (HRF)/translationally controlled tumor protein (TCTP) in a sample from a subject, wherein an amount of HRF/TCTP in the sample greater than normal diagnoses the subject as having or at risk of a food allergy.

In one aspect, HRF measuring includes determining the amount of HRF/TCTP protein or nucleic acid encoding HRF/TCTP (RNA, cDNA) in the sample. In another aspect, HRF measuring includes contacting the sample with an agent or tag (e.g., a detectable agent or tag, such as an antibody, protein or nucleic acid that binds to HRF/TCTP protein or nucleic acid encoding HRF/TCTP) that binds to HRF/TCTP protein or nucleic acid encoding HRF/TCTP and ascertaining the amount of HRF/TCTP protein or nucleic acid encoding HRF/TCTP, or the amount of agent or tag (e.g., a detectable agent or tag, such as an antibody, protein or nucleic acid that binds to HRF/TCTP protein or nucleic acid encoding HRF/TCTP) bound to the HRF/TCTP protein or nucleic acid encoding HRF/TCTP.

The disclosure also provides cell-free (e.g., in solution, in solid phase) and cell-based (e.g., in vitro or in vivo) methods of diagnosing and monitoring progression of a subject having or at increased risk of having a food allergy, allergic reaction, hypersensitivity, inflammatory response, or inflammation, the location, presence or extent of a food allergy, allergic reaction, hypersensitivity, inflammatory response, or inflammation, as well as identifying a subject appropriated for treatment with an HRF sequence, or an antibody that binds to HRF, due to increased probability of responding to treatment. The methods can be performed in solution, in vitro using a biological material or sample, for example, a sample or biopsy of cells, tissue or organ. The methods can also be performed in vivo, for example, in an animal.

In one embodiment, a method includes contacting a biological material or sample (e.g., from a subject) with an HRF sequence, or an antibody that binds to HRF; and assaying for the presence of HRF. The binding to HRF can be used to ascertain the presence or amount of HRF, which can be correlated with increased risk of having a food allergy, allergic reaction, hypersensitivity, inflammatory response, or inflammation, thereby diagnosing the subject. The presence or amount of HRF can also identify a subject appropriate for an anti-HRF treatment, as such subjects will have a greater probability of favorably responding to treatment of a food allergy, allergic reaction, hypersensitivity, inflammatory response, or inflammation, for example, treatment with an HRF sequence (HRF polypeptide or inhibitory nucleic acid) or an anti-HRF antibody. In one aspect, a biological material or sample is obtained from a mammal (e.g., a human). Methods of monitoring progression of a food allergy, allergic reaction, hypersensitivity, inflammatory response, or inflammation can be performed at a regular or irregular intervals, for example, daily, bi-weekly, weekly, bi-monthly, monthly, quarterly, semi- or bi-annually, annually, etc., as appropriate.

Diagnostic methods can be performed on any subject, such as a mammal (e.g., human, primate). Such subjects can have or be at risk of having a condition or disorder associated with HRF activity, function, or expression as set forth herein. For example, a subject can have or be at risk of having a food allergy, allergic reaction, hypersensitivity, inflammatory response or inflammation.

The terms “assaying” and “measuring” and grammatical variations thereof are used interchangeably herein and refer to either qualitative or quantitative determinations, or both qualitative and quantitative determinations. When the terms are used in reference to binding or detection, any means of assessing the relative amount, affinity or specificity of binding is contemplated, including the various methods set forth herein and known in the art. For example, HRF binding can be assayed or measured by an ELISA assay, Western blot or immunoprecipitation assay, or by modulating an activity, function or expression of a native HRF. In another example, antibody binding can be assayed or measured by an ELISA assay, Western blot or immunoprecipitation assay.

The term “correlating” and grammatical variations thereof refers to a relationship or link between two or more entities. For example, as disclosed herein HRF is associated with, among other things, food allergy, allergic reaction, hypersensitivity, inflammatory response or inflammation. Thus, because of this relationship between HRF and a food allergy, allergic reaction, hypersensitivity, inflammatory response or inflammation they correlate with each other. Thus, correlating the presence or quantity of HRF can indicate susceptibility, or the presence and/or extent, or severity of a food allergy, allergic reaction, hypersensitivity, inflammatory response or inflammation in a subject, for example.

In accordance with the disclosure, there are provided methods of identifying an agent that modulates (e.g., reduces or inhibits) an allergy (e.g., a food allergy or an allergic reaction), hypersensitivity, inflammatory response or inflammation. In one embodiment, a method includes contacting histamine releasing factor (HRF)/translationally controlled tumor protein (TCTP) with a test compound in the presence of an immunoglobulin that binds to HRF/TCTP; and determining if the compound inhibits or reduces binding of HRF/TCTP to the immunoglobulin. A reduction or inhibition of binding identifies the test compound as an agent that reduces or inhibits a food allergy, allergic reaction, hypersensitivity, inflammatory response or inflammation. In particular aspects, an immunoglobulin is an IgE, IgG, IgA, IgM or IgD.

The disclosure provides kits including compositions of the disclosure (e.g., HRF polypeptides, antibodies that bind to HRF, nucleic acids encoding HRF sequences or hybridizing sequences, etc.), combination compositions and pharmaceutical formulations thereof, packaged into suitable packaging material. Kits can be used in various methods. For example, a kit can determine an anti-N19 antibody or an anti-HRF antibody that recognizes epitopes outside the N19 portion, since anti-N19 antibody is believed to inhibit HRF/Ig interactions, but the latter anti-HRF antibody might not have the same activity. If so, the ratio of anti-N-19 over anti-HRF (outside of N19) in blood or other body fluids would indicate a contribution of HRF/Ig interaction to the disease.

A kit typically includes a label or packaging insert including a description of the components or instructions for use in vitro, in vivo, or ex vivo, of the components therein. A kit can contain a collection of such components, e.g., HRF sequence, antibody that binds to HRF, alone, or in combination with another therapeutically useful composition (e.g., an immune modulatory drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the disclosure can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Exemplary instructions include, instructions for treating an undesirable or aberrant immune response, immune disorder, immune disease, such as a food allergy, allergic reaction, hypersensitivity, inflammatory response or inflammation. Kits of the disclosure therefore can additionally include labels or instructions for practicing any of the methods of the disclosure described herein including treatment, or diagnostic methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Disclosure kits can additionally include other components. Each component of the kit can be enclosed within an individual container and all of the various containers can be within a single package. Disclosure kits can be designed for cold storage. Disclosure kits can further be designed to contain host cells expressing peptides or antibodies of the disclosure, or that contain encoding nucleic acids. The cells in the kit can be maintained under appropriate storage conditions until the cells are ready to be used. For example, a kit including one or more cells can contain appropriate cell storage medium so that the cells can be thawed and grown.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the disclosure. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a ranges, such as 1-2, 5-10, 10-50, 50-100, 100-500, 100-1000, 500-1000, 1000-2000, 1000-5000, etc. In a further example, reference to a range of K_{D} 10⁻⁵ M to about K_{D} 10⁻¹³ M includes any numerical value or range within or encompassing such values, such as 1×10⁻⁵ M, 1×M10⁻⁶ M, 1×10⁻⁷M, 1×10⁻⁸M, etc.

As also used herein a series of range formats are used throughout this document. The use of a series of ranges includes combinations of the upper and lower ranges to provide a range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### Modes for Carrying Out the Disclosure

Also provided herein is a method for identifying a subject suffering from an allergy likely to respond to an allergen immunotherapy, comprising, or alternatively consisting essentially of, or yet further consisting of, contacting a sample isolated from the subject with an agent that detects an HRF-reactive Ig molecule, and detecting the amount of HRF-reactive Ig molecule in the sample. Non-liming examples of allergies for the method include extrinsic or intrinsic bronchial asthma; Allergic rhinitis; onchocercal dermatitis; atopic dermatitis; eczema; rash; allergic urticaria (e.g. hives); allergic conjunctivitis; drug reactions; Nodules, eosinophilia, rheumatism, dermatitis, and swelling (NERDS); eosophageal and a gastrointestinal allergy. A non-limiting examples of agents that detect an HRF-reactive Ig molecule include a modified HRF molecule (e.g., a modified HRF monomer, a modified HRF dimer, or a modified HRF multimer), in which the modified HRF molecule optionally comprises a C28A mutation, a C172A mutation, or a combination thereof, and an antibody that specifically bind to the HRF-reactive Ig molecule, that is or isn't detectably labeled. Labels for use in the method include a radioactive material, such as a radioisotope, a metal or a metal oxide, examples of such are provided herein. HRF-reactive Ig molecules that are detected include, for example, the antibody or subsequence isotype comprising, or alternatively consisting essentially of, or yet further consisting of, an IgM, IgG, IgA, IgD or IgE isotype.

In some embodiments, also disclosed herein is a method for identifying or assessing whether a subject is likely to respond to a therapy for treatment of an allergy, hypersensitivity, asthma, inflammatory response or inflammation, which comprises contacting a sample isolated from the subject with an agent that detects an HRF monomer, an HRF dimer, an HRF multimer, or an HRF-reactive immunoglobulin (Ig) molecule; and detecting an amount of the HRF monomer, the HRF dimer, the HRF multimer, or the HRF-reactive Ig molecule in the sample. In some cases, also provided herein is a method for diagnosing or determining the severity of a condition selected from an allergy, hypersensitivity, asthma, inflammatory response or inflammation in a subject, which comprises contacting a sample isolated from the subject with an agent that detects an HRF monomer, an HRF dimer, an HRF multimer, or an HRF-reactive immunoglobulin (Ig) molecule; and detecting an amount of the HRF monomer, the HRF dimer, the HRF multimer, or the HRF-reactive Ig molecule in the sample. In additional cases, further provided herein is a method of monitoring a therapy for treatment of an allergy, hypersensitivity, asthma, inflammatory response or inflammation in a subject in need thereof, comprising contacting a sample isolated from the subject with an agent that detects an HRF monomer, an HRF dimer, an HRF multimer, or an HRF-reactive immunoglobulin (Ig) molecule to determine the presence and amount of the HRF monomer, the HRF dimer, the HRF multimer, or the HRF-reactive Ig molecule in the sample. A non-limiting examples of agents that detect an HRF-reactive Ig molecule include a modified HRF molecule (e.g., a modified HRF monomer, a modified HRF dimer, or a modified HRF multimer), in which the modified HRF molecule optionally comprises a C28A mutation, a C172A mutation, or a combination thereof, and an antibody that specifically bind to the HRF-reactive Ig molecule, that is or isn't detectably labeled. Labels for use in the method include a radioactive material, such as a radioisotope, a metal or a metal oxide, examples of such are provided herein. HRF-reactive Ig molecules that are detected include, for example, the antibody or subsequence isotype comprising, or alternatively consisting essentially of, or yet further consisting of, an IgM, IgG, IgA, IgD or IgE isotype.

Non-limiting examples of methods to detect an HRF-reactive Ig molecule include ELISA, affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

The method can be performed on samples from a subject include a body fluid, a lavage sample, blood, plasma, a nasal fluid, tears or saliva, and biopsy of cells, tissue or organ.

For the purpose of this method, the subject is a mammal, for example a human patient, e.g., an adult, or juvenile or an infant, a simian, a murine, a canine, a leporid, such as a rabbit, livestock, sport animals, and pets.

Non-limiting examples of allergen immunotherapies include oral immunotherapy (OIT), sublingual immunotherapy (SLIT), epicutaneous immunotherapy (EPIT), and subcutaneous immunotherapy (SCIT).

As used herein, the term an “oral immunotherapy” or “OTT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OTT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OTT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

The method can further comprise, or alternatively consist essentially of, or yet further consist of isolating the sample from the subject.

As used herein, the term “likely to respond to an allergen therapy” intends a subject likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

In one aspect, the method can further comprise, or alternatively consist essentially of, or yet further consist of detecting a high level of HRF-reactive IgE to identify the subject as likely responsive to the immunotherapy (e.g., an allergy immunotherapy). For example, a high level of HRF-reactive IgE is a positive diagnosis for food allergy or indicative of food allergy severity. In an additional example, a high level of HRF-reactive IgE is a positive diagnosis for asthma or indicative of asthma severity. As used herein, the term “high levels of HRF-reactive IgE” refers to at least about 200 ng/ml, or alternatively at least about 225, or at least about 250, or at least about 300, or at least about 325, or at least about 350, or at least about 375, or at least about 400, or at least about 425, or at least about 450, all measured in ng per ml RE-1 IgE. RE-1 IgE is one of the two known human HRF-reactive IgEs. One can detect the HRF-reactive IgE using methods such as affinity purification, non-denaturing gel purification, HPLC or RP-HPLC, size exclusion, purification on protein A column, or any combination of these techniques, and reagents such as anti-IgE antibody.

In a further aspect, the method can further comprise, or alternatively consist essentially of, or yet further consist of detecting a low level of the HRF-reactive IgE to identify the subject as less likely to be responsive to the therapy. As used herein, the term “low levels of IgE” refers to no more than about 150 ng/ml, or alternative no more than about 125, or alternative no more than about 100, or alternative no more than about 75, or alternatively no more than about 50 ng per ml HE-1 IgE. HE-1 IgE is one of the two known human HRF-reactive IgEs. One can detect the HRF-reactive IgE using methods such as affinity purification, non-denaturing gel purification, HPLC or RP-HPLC, size exclusion, purification on protein A column, or any combination of these techniques, and reagents such as anti-IgE antibody. As used herein, the term a subject less likely to respond to an allergen therapy intends a subject less likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

In an additional aspect, a low level of HRF-reactive IgE during or after treatment identifies the subject as responsive to the therapy. For example, a reduction in levels of HRF reactive IgE during a therapy (e.g., an OTT therapy) is indicative of responsiveness.

In some cases, a high level of HRF-reactive IgE after treatment identifies the subject as not responsive to the therapy. For example, an increase in levels of HRF reactive IgE after a therapy (e.g., an OTT therapy) is indicative of non-responsiveness.

In some embodiments, a low level of HRF-reactive IgG is a positive diagnosis for asthma or indicative of asthma severity. As used herein, the term “low levels of HRF-reactive IgG” refers to no more than about 200 HG Unit mg/ml HRF-reactive IgG or an equivalent thereof as measured prior to the onset of an allergic inflammation.

In some cases, a high level of HRF-reactive IgG is a positive diagnosis for food allergy. As utilized herein, the term “high levels of HRF-reactive IgG” refers to at least about 201 HG Unit mg/ml HRF-reactive IgG or an equivalent thereof as measured prior to the onset of an allergic inflammation.

Also provided herein is a method for monitoring allergen immunotherapy in a subject, the method comprising, or alternatively consisting essentially of, or yet further consisting of, contacting a sample isolated from the subject with an agent that detects an HRF-reactive Ig molecule, and detecting the amount of HRF-reactive Ig molecule in the sample. Allergen immunotherapies that can be monitored include, for example, oral immunotherapy (OIT), sublingual immunotherapy (SLIT), epicutaneous immunotherapy (EPIT), and subcutaneous immunotherapy (SCIT). As used herein, the term an “oral immunotherapy” or “OTT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OIT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OIT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

Non-limiting examples of agents that detect an HRF-reactive Ig molecule include an antibody that specifically bind to the HRF-reactive Ig molecule, that is or isn't detectably labeled. One can detect the HRF-reactive IgE using methods such as affinity purification, non-denaturing gel purification, HPLC or RP-HPLC, size exclusion, purification on protein A column, or any combination of these techniques. For the purpose of this method, the subject is a mammal, for example a human patient, e.g., an adult, or juvenile or an infant. HRF-reactive Ig molecules include, for example, IgM, IgG, IgA, IgD or IgE isotype. The method can further comprise or alternatively consist essentially of, or yet further consist of isolating the sample from the subject.

For the purpose of this method, the term a subject likely to respond to an allergen therapy intends a subject likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

In one aspect, the method can further comprise, or alternatively consist essentially of, or yet further consist of detecting a low level of HRF-reactive IgE or a low level of HRF-reactive IgG indicating effectiveness of the allergen immunotherapy in treating the subject. As used herein, the term “low levels of IgE” refers to no more than about 150 ng/ml, or alternative no more than about 125, or alternative no more than about 100, or alternative no more than about 75, or alternatively no more than about 50 ng per ml RE-1 IgE. RE-1 IgE is one of the two known human HRF-reactive IgEs. Low levels of HRF-reactive IgG refer to no more than about 200 HG Unit mg/ml HRF-reactive IgG or an equivalent thereof as measured prior to the onset of allergic inflammation. One can detect the HRF-reactive IgE or HRF-reactive IgG using methods such as affinity purification, non-denaturing gel purification, HPLC or RP-HPLC, size exclusion, purification on protein A column, or any combination of these techniques, and reagents such as anti-IgE antibody. As used herein, the term a subject less likely to respond to an allergen therapy intends a subject less likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

An amount sufficient or an amount effective can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, status of the disorder, disease or condition treated or the side effects of treatment. In addition, an amount sufficient or an amount effective need not be sufficient or effective if given in single or multiple doses without a second composition (e.g., agent), treatment, protocol or therapeutic regimen, since additional doses, amounts or duration above and beyond such doses, or additional compositions (e.g., agents), treatments, protocols or therapeutic regimens may be included in order to be considered effective or sufficient in a given subject. Amounts considered sufficient also include amounts that result in a reduction of the use of another treatment, therapeutic regimen or protocol.

An amount sufficient or an amount effective need not be effective in each and every subject treated, prophylactically or therapeutically, nor a majority of treated subjects in a given group or population. An amount sufficient or an amount effective means sufficiency or effectiveness in a particular subject, not a group or the general population. As is typical for such methods, some subjects will exhibit a greater response, or less or no response to a treatment method.

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. Clinical indications of treatment can include in some aspect, a reduction in diarrhea is an indication of success of treatment of a food allergy. Hypothermia and reduced physical activity (or mobility) are typical signs of anaphylaxis (which can be seen in food allergy and other allergic diseases) and can serve as a marker of allergic severity or a treatment. Thus, indication of normal temperature of increased physical activity are indications of successful treatment. Sub-clinical evidence of cytokines (see, e.g., FIG. 24) IL-4, IL-5, and IL-13, IL-25, IL-33, TSLP, IL-1beta, IL-6 and TNF, can be measured as an indication of disease severity and treatment.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

In a further aspect, the method can further comprise, or alternatively consist essentially of, or yet further consist of detecting a high level of the HRF-reactive IgE indicating the allergen immunotherapy is not effective in treating the subject. As used herein, the term “high levels of HRF-reactive IgE” refers to at least about 200 ng/ml, or alternatively at least about 225, or at least about 250, or at least about 300, or at least about 325, or at least about 350, or at least about 375, or at least about 400, or at least about 425, or at least about 450, all measured in ng per ml RE-1 IgE. RE-1 IgE is one of the two known human HRF-reactive IgEs. Low levels of HRF-reactive IgG refer to no more than about 200 HG Unit mg/ml HRF-reactive IgG or an equivalent thereof as measured prior to the onset of allergic inflammation. One can detect the HRF-reactive IgE or HRF-reactive IgG using methods such as affinity purification, non-denaturing gel purification, HPLC or RP-HPLC, size exclusion, purification on protein A column, or any combination of these techniques, and reagents such as anti-IgE antibody. As used herein, the term a subject less likely to respond to an allergen therapy intends a subject less likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition. Exemplary oral immunotherapies (OIT) include for example subcutaneous immunotherapy (SIT), or sublingual immunotherapy (SLIT) as the allergen immunotherapy.

As used herein, the term an “oral immunotherapy” or “OTT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OIT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OIT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

As used herein, the term “assessing” in the context of assessing whether a subject is likely to respond to a therapy is used interchangeably with “identifying” and refers to the selection of a subject that can potentially benefit from a treatment therapy. In some instances, the subject may be suspected of having an allergy, hypersensitivity, asthma, inflammatory response or inflammation or has an allergy, hypersensitivity, asthma, inflammatory response or inflammation and can undergo an assessment process to determine whether the subject can benefit from a specific treatment plan.

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. Clinical indications of treatment can include in some aspect, a reduction in diarrhea is an indication of success of treatment of a food allergy. Hypothermia and reduced physical activity (or mobility) are typical signs of anaphylaxis (which can be seen in food allergy and other allergic diseases) and can serve as a marker of allergic severity or a treatment. Thus, indication of normal temperature of increased physical activity are indications of successful treatment. Sub-clinical evidence of cytokines (see, e.g., FIG. 24) IL-4, IL-5, and IL-13, IL-25, IL-33, TSLP, IL-1beta, IL-6 and TNF, can be measured as an indication of disease severity and treatment.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

In another aspect, the methods can further comprise, or alternatively consist essentially of, or yet further consist of, contacting the sample with an agent that detects an HRF/HRF-reactive Ig complex under conditions for the formation of an HRF/HRF-reactive Ig complex, and detecting the level of the complex in the sample. Non-limiting examples of samples from a subject include a body fluid or lavage sample, blood, plasma, nasal fluids, tears or saliva, and biopsy of cells, tissue or organ. Non-limiting examples of agents that detect an HRF-reactive Ig molecule include an antibody that specifically bind to the HRF-reactive Ig molecule, that is or isn't detectably labeled. HRF-reactive Ig molecules include, for example, the antibody or subsequence isotype comprising, or alternatively consisting essentially of, or yet further consisting of, an IgM, IgG, IgA, IgD or IgE isotype. Non-limiting examples of suitable techniques that detect an HRF/HRF-reactive Ig complex include ELISA, affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

In a further aspect, the methods can further comprise, or alternatively consist essentially of, or yet further consist of administering an effective amount of the allergen immunotherapy. Non-limiting examples of allergen immunotherapy include oral immunotherapy (OTT), sublingual immunotherapy (SLIT), epicutaneous immunotherapy (EPIT), and subcutaneous immunotherapy (SCIT), examples of which are provided herein.

As used herein, the term an “oral immunotherapy” or “OTT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OIT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OIT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

In one aspect, the methods can further comprise, or alternatively consist essentially of, or yet further consist of administering allergen immunotherapy in combination with an inhibitor of binding of HRF to an Ig molecule. Non-limiting examples of agents that inhibit binding of HRF to an Ig molecule include peptides and polypeptides, such as HRF sequences, HRF subsequences or fragments (e.g., a sequence that binds to an Ig, such as an IgE), antibodies and antibody subsequences (e.g., polyclonal or monoclonal and any of IgM, IgG, IgA, IgD or IgE isotypes) known to the skilled artisan and as set forth herein. Such sequences can be mammalian, humanized, human or chimeric.

Particular examples include a fragment of HRF/TCTP polypeptide that binds to an immunoglobulin, such as an IgE. An exemplary HRF sequence includes or consists of amino acids 1-19 or amino acids 79-142 of a mammalian HRH/TCTP sequence, for example, all or a portion of a MIIYRDLISHDEMFSDIYK (SEQ ID NO:1) sequence, or all or a portion of a QETSFTKEAYKKYIKDYMKSIKGKLEEQRPERVKPFMTGAAEQIKHILANFKNYQFFIGENMNP (SEQ ID NO:2) sequence. Particular non-limiting examples of HRF binding antibodies include commercial antibodies from Santa Cruz Biotechnology, Inc. (Santa Cruz, Calif.), as set forth in Table 2. Additional particular non-limiting examples of HRF binding antibodies include commercial antibodies from Assay Designs/Stressgen (Ann Arbor, Mich.), Proteintech Group (Chicago, Ill.), R and D Systems, Inc. (Minneapolis, Minn.), Sigma-Aldrich Corp. (St. Louis, Mo.), AbDSerotec/MorphoSys UK Ltd. (Oxford, UK), Strategic Diagnostics (SDIX) (Newark, Del.), Abcam (Cambridge, Mass.) and Novus Biologicals, LLC (Littleton, Colo.) as set forth in Table 3.

In a further aspect, the methods can further comprise, or alternatively consist essentially of, or yet further consist of administering an allergen immunotherapy in combination with an inhibitor, effective to treat the condition. Non-limiting examples of such include a peptide or polypeptide that inhibits the binding of an HRF monomer, an HRF dimer, or a HRF multimer, each with an HRF-reactive immunoglobulin (Ig). Non-limiting examples of allergen immunotherapy include for example oral immunotherapy (OTT), sublingual immunotherapy (SLIT), epicutaneous immunotherapy (EPIT), and subcutaneous immunotherapy (SCIT).

As used herein, the term an “oral immunotherapy” or “OTT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OTT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OTT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

In one aspect, the methods can further comprise, or alternatively consist essentially of, or yet further consist of administering the allergen immunotherapy prior to, or after the sample is isolated from the subject. The allergen immunotherapy can be administered by any suitable route and may be practiced via systemic, regional or local administration, by any route. For example, an HRF sequence or an antibody that binds to HRF may be administered systemically, regionally or locally, via ingestion, via inhalation, topically, intravenously, orally (e.g., ingestion or inhalation), intramuscularly, intraperitoneally, intradermally, subcutaneously, intracavity, intracranially, transdermally (topical), parenterally, e.g. transmucosally or rectally. Compositions and methods of the invention including pharmaceutical formulations can be administered via a (micro) encapsulated delivery system or packaged into an implant for administration. It will also be appreciated that the preferred route will vary with the condition and age of the recipient, and the disease being treated. Methods of determining the most effective means and dosage of administration are known to those of skill in the art and will vary with the composition used for therapy, the purpose of the therapy, and the subject being treated. Single or multiple administrations can be carried out with the dose level and pattern being selected by the treating physician. Suitable dosage and methods of administering the agents are known in the art.

Also provided herein is a method to monitor therapy for treatment of allergic reaction, hypersensitivity, asthma, inflammatory response or inflammation in a subject, comprising, or alternatively consisting essentially of, or yet further consisting of, detecting the level of HRF monomer, HRF dimer, HRF multimer, or HRF-reactive Ig molecule in a sample isolated from the subject, the method comprising, or alternatively consisting essentially of, or yet further consisting of, contacting the sample with an agent that detects HRF monomer, HRF dimer, HRF multimer, or HRF-reactive Ig molecule. Non-limiting examples of therapy or allergen immunotherapies include oral immunotherapy (OIT), sublingual immunotherapy (SLIT), epicutaneous immunotherapy (EPIT), and subcutaneous immunotherapy (SCIT).

As used herein, the term an “oral immunotherapy” or “OIT” comprises, or alternatively consists essentially of, or yet further consists of at least two phases of ingestion of the allergen: (1) Phase 1 comprising, or alternatively consisting essentially of, or yet further consisting of upscaling patient exposure to allergen until desensitization is reached. In some cases, OIT upscaling comprises an initial escalation step followed by gradual build-up of allergen until target dose is achieved. The initial escalation step comprises, or alternatively consists of, rapidly administering increasing concentrations of allergen, with at least more than one dose of allergen within a single day, beginning with a small dose that is rapidly increased, non-limiting examples include rush immunotherapy or cluster immunotherapy. The build-up stage comprises gradually increasing allergen dosage under observation until a target dose that achieves allergen desensitization is reached. In some cases, the initial escalation phase is rush immunotherapy, with at least more than one dose of allergen given rapidly during a single day beginning with a small dose that is rapidly increased. (2) Phase 2 comprises daily maintenance dosing of allergen at levels that sustain allergen desensitization.

In other exemplary embodiments, OIT comprises, or alternatively consists essentially of, or yet further consists of at least three phases of ingestion of the allergen: (1) phase 1 comprising, or alternatively consisting essentially of, or yet further consisting of an initial escalation phase with at least more than one dose of allergen within a single day, beginning with a small dose that is rapidly increased, non limiting examples of this method include rush immunotherapy or cluster immunotherapy; (2) phase 2 comprises build-up dosing gradually under observation until a target dose is reached; and (3) phase 3 comprises daily maintenance dosing.

Non-limiting example of sublingual immunotherapy (SLIT), comprises, or alternatively consists essentially of, or yet further consists of immunotherapy in which patients take gradually increased doses of allergen extract that are placed under the tongue and then spit or swallowed. Exemplary SLIT protocol comprises, or alternatively consists essentially of, or yet further consists of two phases 1) phase 1 escalation phase comprising, or alternatively consisting essentially of, or yet further consisting of building up dosing gradually under observation until a target dose is reached; and (2) phase 2 comprises maintenance dosing. In a further aspect, in SLIT immunotherapy, the allergen is delivered sublingually in a liquid form and then held under the tongue for at least a minute and swallowed. In another aspect, SLIT doses start with at least about 1-microgram levels of the allergenic protein and increase to about 10 mg by maintenance phase. In a representative SLIT study by Fleischer et al., 40 adolescents and adults, who were allergic to peanut, were treated with peanut SLIT or placebo, with a maximum dose of 1.4 mg for 44 weeks (45). After undergoing this 44 week SLIT treatment, the subjects were challenged with 5000 mg oral food challenge. 14 out of 20 subjects receiving peanut SLIT therapy were found to be less allergic compared with 3 out of 20 subjects in the placebo group (P<0.001). The responders were found to tolerate a median dose of 496 mg in the oral food challenge, a significant increase from 3.5 mg prior to the study. In the peanut SLIT group, 44, 63.1% were symptom free. Other research groups have carried out similar SLIT studies and obtained similarly successful results (46-48).

Non-limiting example of epicutaneous immunotherapy (EPIT) comprises, or alternatively consists essentially of, or yet further consists of immunotherapy with delivery of allergen to the skin through application of an allergen-containing patch designed to activate skin Langerhans cells, with subsequent migration to lymph nodes and downregulation of effector cell responses. A representative EPIT with human subjects included 54 children with severe peanut allergy (age, 5-17 years) who were all treated with the peanut patch containing 100 μg of peanut protein after 6 months of blinded therapy (53). Oral food challenges were conducted every six months. After 12-18 months, the children showed consistent and sustained desensitization, with up to 67% responders at 18 months reaching 1.1 to 2.5 g of peanut protein tolerance (approximately 3.3-8 peanuts).

Non-limiting example of “subcutaneous immunotherapy” (SCIT) comprises or alternatively consists essentially of, or yet further consists of treating a subject with small doses of allergens administered subcutaneously or by injection. In one aspect, the allergen dose comprises about 3 μg to about 23 μg allergen per injection (54).

Non-limiting examples of agents that detect an HRF-reactive Ig molecule include an antibody that specifically bind to the HRF-reactive Ig molecule, that is or isn't detectably labeled. Non-limiting examples of agents that detect an HRF monomer, an HRF dimers, or HRF multimers include an antibody that specifically bind to the HRF monomer, the HRF dimer, or HRF multimer molecule, that is or isn't detectably labeled. Non-limiting examples of samples from a subject include fluid or lavage sample, blood or plasma, body fluids, nasal fluids, tears or saliva, and biopsy of cells, tissue or organ. For the purpose of this method, the subject is a mammal, for example a human patient, e.g., an adult, or juvenile or an infant, a simian, a murine, a canine, a leporid, such as a rabbit, livestock, sport animals, and pets. HRF-reactive Ig molecules include, for example, the antibody or subsequence isotype comprising, or alternatively consisting essentially of, or yet further consisting of, an IgM, IgG, IgA, IgD or IgE isotype.

Also provided herein is a method of identifying a subject that will or is likely to respond to therapy for treatment of allergic reaction, hypersensitivity, asthma, inflammatory response or inflammation in, comprising, or alternatively consisting essentially of, or yet further consisting of detecting the level of HRF monomer, HRF dimer, HRF multimer, or HRF-reactive Ig molecule in a sample isolated from the subject, the method comprising, or alternatively consisting essentially of, or yet further consisting of, contacting the sample with an agent that detects HRF monomer, HRF dimer, HRF multimer, or HRF-reactive Ig molecule. One can detect the HRF-reactive IgE or HRF-reactive Ig or HRF monomer or HRF dimers or HRF multimers using methods such as affinity purification, non-denaturing gel purification, HPLC or RP-HPLC, size exclusion, purification on protein A column, or any combination of these techniques, For the purpose of this method, the subject is a mammal, for example a human patient, e.g., an adult, or juvenile or an infant, a simian, a murine, a canine, a leporid, such as a rabbit, livestock, sport animals, and pets.

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. Clinical indications of treatment can include in some aspect, a reduction in diarrhea is an indication of success of treatment of a food allergy. Hypothermia and reduced physical activity (or mobility) are typical signs of anaphylaxis (which can be seen in food allergy and other allergic diseases) and can serve as a marker of allergic severity or a treatment. Thus, indication of normal temperature of increased physical activity are indications of successful treatment. Sub-clinical evidence of cytokines (see, e.g., FIG. 24) IL-4, IL-5, and IL-13, IL-25, IL-33, TSLP, IL-1beta, IL-6 and TNF, can be measured as an indication of disease severity and treatment.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

As used herein, the term a subject likely to respond to an allergen therapy intends a subject likely to show a detectable improvement in condition. Non-limiting examples of a detectable improvement include a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Also provided herein is a method for diagnosing or determining the severity of a condition selected from the group of: allergic reaction, hypersensitivity, asthma, inflammatory response or inflammation/or severity of allergic reaction, or inflammation in a subject, comprising, or alternatively consisting essentially of, or yet further consisting of, detecting the level of HRF monomer, HRF dimer, HRF multimer, or HRF-reactive Ig molecule in a sample isolated from the subject, the method comprising, or alternatively consisting essentially of, or yet further consisting of contacting the sample with an agent that detects HRF monomer, HRF dimer, HRF multimer, or HRF-reactive Ig molecule. Non-limiting examples of agents that detect an HRF-reactive Ig molecule include an antibody that specifically bind to the HRF-reactive Ig molecule, that is or isn't detectably labeled. Non-limiting examples of agents that detect an HRF monomer, an HRF dimers, or HRF multimers include an antibody that specifically bind to the HRF monomer, the HRF dimer, or HRF multimer molecule, that is or isn't detectably labeled. One can detect the HRF-reactive IgE or HRF-reactive IgG using methods such as affinity purification, non-denaturing gel purification, HPLC or RP-HPLC, size exclusion, purification on protein A column, or any combination of these techniques, Non-limiting examples of samples from a subject include a body fluid or lavage sample, blood, plasma, body fluids, nasal fluids, tears or saliva, and biopsy of cells, tissue or organ. For the purpose of this method, the subject is a mammal, for example a human patient, e.g., an adult, or juvenile or an infant, a simian, a murine, a canine, a leporid, such as a rabbit, livestock, sport animals, and pets. HRF-reactive Ig molecules include, for example, the antibody or subsequence isotype comprising, or alternatively consisting essentially of, or yet further consisting of, an IgM, IgG, IgA, IgD or IgE isotype.

As used herein, the terms “treating,” “treatment” and the like mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. In one aspect, the term “treatment” excludes prophylaxis. Clinical indications of treatment can include in some aspect, a reduction in diarrhea is an indication of success of treatment of a food allergy. Hypothermia and reduced physical activity (or mobility) are typical signs of anaphylaxis (which can be seen in food allergy and other allergic diseases) and can serve as a marker of allergic severity or a treatment. Thus, indication of normal temperature of increased physical activity are indications of successful treatment. Sub-clinical evidence of cytokines (see, e.g., FIG. 24) IL-4, IL-5, and IL-13, IL-25, IL-33, TSLP, IL-1beta, IL-6 and TNF, can be measured as an indication of disease severity and treatment.

As used herein, to “treat” further includes systemic amelioration of the symptoms associated with the pathology and/or a delay in onset of symptoms. Clinical and sub-clinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disorder, disease or condition, such as one or more adverse symptoms, disorders, illnesses, pathologies, diseases, or complications caused by or associated with the food allergy, allergic reaction, hypersensitivity, inflammatory response or inflammation, or an improvement in an underlying cause or a consequence of the disorder, disease or condition, or a reversal of the disorder, disease or condition.

Treatment can therefore result in decreasing, reducing, inhibiting, suppressing, limiting, controlling or preventing a disorder, disease or condition, or an associated symptom or consequence, or underlying cause; decreasing, reducing, inhibiting, suppressing, limiting, controlling or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disorder, disease or symptom in the subject, such as one or more adverse symptoms, disorders, illnesses, pathologies, diseases, or complications caused by or associated with a food allergy, allergic reaction, hypersensitivity, inflammatory response or inflammation. Treatment methods affecting one or more underlying causes of the condition, disorder, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the food allergy, allergic reaction, hypersensitivity, inflammatory response or inflammation, over a short or long duration of time (hours, days, weeks, months, etc.).

Also provided herein is a method of identifying a subject that will or is likely to respond to therapy for treatment of allergic reaction, hypersensitivity, asthma, inflammatory response or inflammation in, comprising, or alternatively consisting essentially of, or yet further consisting of detecting the level of HRF monomer, HRF dimer, HRF multimer, or HRF-reactive Ig molecule in a sample isolated from the subject, the method comprising, or alternatively consisting essentially of, or yet further consisting of, contacting the sample with an agent that detects HRF monomer, HRF dimer, HRF multimer, or HRF-reactive Ig molecule. For the purpose of this method, the subject is a mammal, for example a human patient, e.g., an adult, or juvenile or an infant, a simian, a murine, a canine, a leporid, such as a rabbit, livestock, sport animals, and pets. Non-limiting examples of agents that detect an HRF-reactive Ig molecule include an antibody that specifically bind to the HRF-reactive Ig molecule, that is or isn't detectably labeled. Non-limiting examples of agents that detect an HRF monomer, an HRF dimers, or HRF multimers include an antibody that specifically bind to the HRF monomer, HRF dimer, or HRF multimer molecule, that is or isn't detectably labeled. One can detect the HRF-reactive IgE or HRF-reactive IgG using methods such as affinity purification, non-denaturing gel purification, HPLC or RP-HPLC, size exclusion, purification on protein A column, or any combination of these techniques, Non-limiting examples of samples from a subject include fluid or lavage sample, blood or plasma, body fluids, nasal fluids, tears or saliva, and biopsy of cells, tissue or organ. For the purpose of this method, the subject is a mammal, for example a human patient, e.g., an adult, or juvenile or an infant, a simian, a murine, a canine, a leporid, such as a rabbit, livestock, sport animals, and pets. HRF-reactive Ig molecules include, for example, the antibody or subsequence isotype comprising, or alternatively consisting essentially of, or yet further consisting of, an IgM, IgG, IgA, IgD or IgE isotype.

In one aspect, the methods can further comprise contacting the sample with an agent that detects an HRF/HRF-reactive Ig complex under conditions for the formation of an HRF/HRF-reactive Ig complex, and detecting the level of the complex in the sample. Non-limiting examples of agents that detect an HRF monomer, an HRF dimers, or HRF multimers include an antibody that specifically bind to the HRF monomer, HRF dimer, or HRF multimer molecule, that is or isn't detectably labeled. One can detect the HRF-reactive IgE or HRF-reactive IgG using methods such as affinity purification, non-denaturing gel purification, HPLC or RP-HPLC, size exclusion, purification on protein A column, or any combination of these techniques, Non-limiting examples of samples from a subject include fluid or lavage sample, blood or plasma, body fluids, nasal fluids, tears or saliva, and biopsy of cells, tissue or organ. HRF-reactive Ig molecules include, for example, the antibody or subsequence isotype comprising, or alternatively consisting essentially of, or yet further consisting of, an IgM, IgG, IgA, IgD or IgE isotype.

In one aspect, the methods can further comprise, or alternatively consist essentially of, or yet further consist of detecting high levels of HRF monomer, HRF dimer, HRF multimer, or HRF-reactive Ig molecule as a positive diagnosis for allergic reaction, hypersensitivity, inflammatory response or inflammation or its severity and low levels of HRF monomer, HRF dimer, HRF multimer, or HRF-reactive Ig molecule as a negative diagnosis for the allergic reaction, hypersensitivity, inflammatory response or inflammation or its severity. As used herein, the term “high levels of HRF-dimerization or HRF-oligomerization” refers to at least about 500 AU HRF-dimers or at least about 300 ng/g tissue HRF multimers or an equivalent of each thereof as measured prior to the onset of allergic inflammation. As used herein, the term “low levels of HRF-dimerization or HRF-oligomerization” refers to no more than about 501 AU HRF-dimers or no more than about 301 ng/g tissue HRF multimers or an equivalent of each thereof as measured prior to the onset of allergic inflammation. As used herein, the term “low levels of IgE” refers to no more than about 150 ng/ml, or alternative no more than about 125, or alternative no more than about 100, or alternative no more than about 75, or alternatively no more than about 50 ng per ml RE-1 IgE. RE-1 IgE is one of the two known human HRF-reactive IgEs. As used herein, the term “high levels of HRF-reactive IgE” refers to at least about 200 ng/ml, or alternatively at least about 225, or at least about 250, or at least about 300, or at least about 325, or at least about 350, or at least about 375, or at least about 400, or at least about 425, or at least about 450, all measured in ng per ml HE-1 IgE. RE-1 IgE is one of the two known human HRF-reactive IgEs. As used herein, the term “low levels of HRF-reactive IgG” refers to no more than about 200 HG Unit mg/ml HRF-reactive IgG or an equivalent thereof as measured prior to the onset of allergic inflammation. As used herein, the term “high levels of HRF-reactive IgG” refers to at least about 201 HG Unit mg/ml HRF-reactive IgG or an equivalent thereof as measured prior to the onset of allergic inflammation.

In another aspect, the methods can further comprise, or alternatively consist essentially of, or yet further consist of detecting low levels of HRF-reactive IgG molecule as a positive diagnosis for asthma or its severity. As used herein, the term “low levels of HRF-reactive IgG” refers to no more than about 200 HG Unit mg/ml HRF-reactive IgG or an equivalent thereof as measured prior to the onset of allergic inflammation.

In a further aspect, the methods can further comprise, or alternatively consist essentially of, or yet further consist of administering an effective amount of the therapy. In the methods of the invention in which a therapeutic benefit or improvement is a desired outcome, a composition of the invention such as an HRF polypeptide or an antibody that binds to HRF, can be administered in a sufficient or effective amount to a subject in need thereof. An “amount sufficient” or “amount effective” refers to an amount that provides, in single or multiple doses, alone or in combination, with one or more other compositions (therapeutic agents such as a drug), treatments, protocols, or therapeutic regimens agents, a detectable response of any duration of time (long or short term), an expected desired outcome in or a benefit to a subject of any measurable or detectable degree or for any duration of time (e.g., for minutes, hours, days, months, years, or cured). For example, a sufficient amount of an HRF sequence, or an antibody or subsequence that binds to HRF, is considered as having a therapeutic effect if administration results in a decreased or reduced amount or frequency of immunotherapy being required for treatment of a one or more adverse symptoms, disorders, illnesses, pathologies, diseases, or complications caused by or associated with the food allergy, allergic reaction, hypersensitivity, inflammatory response or inflammation. The doses or “sufficient amount” or “effective amount” for treatment (e.g., to provide a therapeutic benefit or improvement) typically are effective to ameliorate a disorder, disease or condition, or one, multiple or all adverse symptoms, consequences or complications of the disorder, disease or condition, one or more adverse symptoms, disorders, illnesses, pathologies, diseases, or complications, for example, caused by or associated with the food allergy, allergic reaction, hypersensitivity, inflammatory response or inflammation to a measurable extent, although decreasing, reducing, inhibiting, suppressing, limiting or controlling a progression or worsening of the disorder, disease or condition or a symptom, is a satisfactory outcome.

In one aspect, the methods can further comprise, or alternatively consist essentially of, or yet further consist of administering prior to, or after the sample is isolated from the subject. In one aspect, the methods can further comprise, or alternatively consist essentially of, or yet further consist of administering an inhibitor of HRF binding to an Ig molecule. In a particular aspect, the methods can further comprise, or alternatively consist essentially of, or yet further consist of administering an inhibitor of HRF binding to an Ig molecule, non-limiting examples of such include a peptide or polypeptide that inhibits the binding of HRF monomer, an HRF dimer, or a HRF multimer with an HRF-reactive Ig. Non-limiting examples of agents that inhibit binding of HRF to an Ig molecule include peptides and polypeptides, such as HRF sequences, HRF subsequences or fragments (e.g., a sequence that binds to an Ig, such as an IgE), antibodies and antibody subsequences (e.g., polyclonal or monoclonal and any of IgM, IgG, IgA, IgD or IgE isotypes) known to the skilled artisan and as set forth herein. Such sequences can be mammalian, humanized, human or chimeric. Particular examples include a fragment of HRF/TCTP polypeptide that binds to an immunoglobulin, such as an IgE. An exemplary HRF sequence includes or consists of amino acids 1-19 or amino acids 79-142 of a mammalian HRH/TCTP sequence, for example, all or a portion of a MIIYRDLISHDEMFSDIYK (SEQ ID NO:1) sequence, or all or a portion of a QETSFTKEAYKKYIKDYMKSIKGKLEEQRPERVKPFMTGAAEQIKHILANFKNYQFFIGENMNP (SEQ ID NO:2) sequence. Particular non-limiting examples of HRF binding antibodies include commercial antibodies from Santa Cruz Biotechnology, Inc. (Santa Cruz, Calif.), as set forth in Table 2. Additional particular non-limiting examples of HRF binding antibodies include commercial antibodies from Assay Designs/Stressgen (Ann Arbor, Mich.), Proteintech Group (Chicago, Ill.), R and D Systems, Inc. (Minneapolis, Minn.), Sigma-Aldrich Corp. (St. Louis, Mo.), AbDSerotec/MorphoSys UK Ltd. (Oxford, UK), Strategic Diagnostics (SDIX) (Newark, Del.), Abcam (Cambridge, Mass.) and Novus Biologicals, LLC (Littleton, Colo.) as set forth in Table 3.

In one aspect, the HRF-reactive Ig in the methods can further comprise, or alternatively consist essentially of, or yet further consist of HRF-reactive IgG or an HRF-reactive IgE. In one aspect, the sample to be selected from in the methods can further comprise, or alternatively consist essentially of, or yet further consist of a sample from body fluids such as blood, plasma, nasal fluids, tears or saliva. In a further aspect, sample comprises a biopsy of cells, tissue or organ. In a further aspect, the HRF in the methods can further comprise, or alternatively consist essentially of, or yet further consist of an HRF monomer, an HRF dimer, or an HRF multimer. In one aspect, the complex in the methods can further comprise, or alternatively consist essentially of, or yet further consist of a detectably labeled complex.

Also provided herein is a method for the treatment of a condition from the group of: allergic reaction, hypersensitivity, asthma, inflammatory response or inflammation in a subject in need thereof, comprising, or alternatively consisting essentially of, or yet further consisting of, administering an effective amount of an agent that interferes with the formation of HRF-Ig complexes to the subject, thereby treating the condition. In one aspect, the agent in the method for the treatment can further comprise, or alternatively consist essentially of, or yet further consist of an agent that binds an HRF-reactive immunoglobulin (Ig). In one aspect, the agent in the peptide or polypeptide of the method can further comprise, or alternatively consist essentially of, or yet further consist of a peptide or polypeptide that inhibits the binding of an HRF monomer, an HRF dimer, or a HRF multimer with an HRF-reactive immunoglobulin (Ig). In a further aspect, the agent in the method can further comprise, or alternatively consist essentially of, or yet further consist of an antibody or an antibody subsequence that inhibits the binding of HRF monomer, an HRF dimer, or an HRF multimer to an HRF-reactive Ig. In one particular aspect, the agent in the method can further comprise, or alternatively consist essentially of, or yet further consist of an antibody or an antibody subsequence that is human or humanized. In one aspect, the agent in the method can further comprise, or alternatively consist essentially of, or yet further consist of an antibody or an antibody subsequence that is monoclonal. In one aspect, the agent in the method can further comprise, or alternatively consist essentially of, or yet further consist of the antibody or antibody subsequence that competes for binding of an antibody set forth in Tables 2 or 3 for binding to the HRF to the HRF-reactive Ig. In one aspect, the agent in the method can further comprise, or alternatively consist essentially of, or yet further consist of the antibody or antibody subsequence capable of binding to an epitope of the HRF to which an antibody set forth in Tables 2 or 3 binds. In another aspect, the agent in the method can further comprise, or alternatively consist essentially of, or yet further consist of the antibody or antibody subsequence that has a binding affinity (Kd) for binding to HRF-Ig complex of about 10⁻⁵ to about 10⁻′³M. In one aspect, the agent in the method can further comprise, or alternatively consist essentially of, or yet further consist of the antibody or antibody subsequence that has a binding affinity (Kd) for binding to the HRF that binds to the HRF-reactive Ig within about 1-1000 of the binding affinity (Kd) of an antibody set forth in Tables 2 or 3. In one aspect, the agent in the method can further comprise, or alternatively consist essentially of, or yet further consist of the antibody or subsequence isotype comprising an IgM, IgG, IgA, IgD or IgE isotype. In another aspect, the agent in the method can further comprise, or alternatively consist essentially of, or yet further consist of the IgG or IgA isotype selected from IgG1, IgG2, IgG3, and IgG4; and IgA1 and IgA2. In one aspect, the agent in the method can further comprise, or alternatively consist essentially of, or yet further consist of the antibody subsequence selected from Fab, Fab′, F(ab′)2, Fv, Fd, single-chain Fv (scFv), disulfide-linked Fvs (sdFv), light chain variable region V_{L}, heavy chain variable region V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, (scFv)₂-Fc and IgG4PE.

In one aspect, the HRF in the method can further comprise, or alternatively consist essentially of, or yet further consist of the HRF/TCTP polypeptide, or a fragment of an HRF/TCTP polypeptide comprising, or alternatively consisting essentially of, or yet further consisting of a fusion or chimeric polypeptide. In a particular aspect, the HRF in the method can further comprise, or alternatively consist essentially of, or yet further consist of the fusion or chimeric polypeptide comprising, or alternatively consisting essentially of, or yet further consisting of an HRF polypeptide, or a fragment of an HRF polypeptide fused to a tag or an immunoglobulin sequence. In one aspect, the agent in the method can further comprise, or alternatively consist essentially of, or yet further consist of the immunoglobulin sequence comprising, or alternatively consisting essentially of, or yet further consisting of an IgG sequence. In another aspect, the HRF in the method can further comprise, or alternatively consist essentially of, or yet further consist of an HRF polypeptide, or a fragment of an HRF polypeptide that binds to an HRF reactive immunoglobulin. In a further aspect, the HRF in the method can further comprise, or alternatively consist essentially of, or yet further consist of a fragment of HRF polypeptide that binds to an HRF-reactive immunoglobulin comprises or consists of amino acids 1-19 or amino acids 79-142 of a mammalian HRF/TCTP sequence. In one aspect, the HRF in the method can further comprise, or alternatively consist essentially of, or yet further consist of the fragment of HRF/TCTP polypeptide that binds to an immunoglobulin comprises or consists of MIIYRDLISHDEMFSDIYK (SEQ ID NO:1) or QETSFTKEAYKKYIKDYMKSIKGKLEEQRPERVKPFMTGAAEQIKHILANFKNYQFFIGENMNP (SEQ ID NO:2) sequence. In another aspect, the HRF in the method can further comprise, or alternatively consist essentially of, or yet further consist of the HRF/TCTP polypeptide comprising, or alternatively consisting essentially of, or yet further consisting of a mammalian HRF/TCTP sequence.

In one aspect, the effective amount in the method can further comprise, or alternatively consist essentially of, or yet further consist of an amount sufficient to protect against allergic reaction, hypersensitivity, asthma, inflammatory response or inflammation/or severity of allergic reaction, inflammation, food allergy, hypersensitivity, inflammatory response, decrease, reduce, inhibit, suppress, limit or control susceptibility to the food allergy, allergic reaction or hypersensitivity, or decrease, reduce, inhibit, suppress, limit or control the food allergy, allergic reaction, hypersensitivity, inflammatory response or inflammation. In another aspect, the effective amount in the method can further comprise, or alternatively consist essentially of, or yet further consist of an amount sufficient to decrease, reduce, inhibit, suppress, limit, control or improve the probability, severity, frequency, or duration of one or more adverse symptoms, disorders, illnesses, pathologies, diseases, or complications caused by or associated with the allergic reaction, hypersensitivity, asthma, inflammatory response or inflammation/or severity of allergic reaction, or inflammation in the subject.

In one aspect, the effective amount in the method can further comprise, or alternatively consist essentially of, or yet further consist of an amount that reduces or inhibits progression, severity, frequency, duration or probability of an adverse symptom of the allergic reaction, hypersensitivity, asthma, inflammatory response or inflammation/or severity of allergic reaction, or inflammation in the subject. In one aspect, the adverse symptoms in the method can further comprise, or alternatively consist essentially of, or yet further consist of diarrhea, shortness of breath (dyspnea), wheezing, stridor, coughing, airway remodeling, rapid breathing (tachypnea), prolonged expiration, runny nose, rapid or increased heart rate (tachycardia), rhonchous lung, over-inflation of the chest or chest-tightness, decreased lung capacity, an acute asthmatic episode, lung, airway or respiratory mucosum inflammation, or lung, airway or respiratory mucosum tissue damage. In another aspect, the allergic reaction in the method can further comprise, or alternatively consist essentially of, or yet further consist of: extrinsic or intrinsic bronchial asthma; allergic rhinitis; onchocercal dermatitis; atopic dermatitis; eczema; rash; allergic urticaria (e.g. hives); allergic conjunctivitis; drug reactions; nodules, eosinophilia, rheumatism, dermatitis, and swelling (NERDS); eosophageal and a gastrointestinal allergy. In another aspect, wherein the hypersensitivity, inflammatory response or inflammation in the method comprises, or alternatively consists essentially of, or yet further consists of a respiratory disease or disorder.

In one aspect, the respiratory disease or disorder in the method can comprise, or alternatively consist essentially of, or yet further consist of a respiratory disease or disorder that affects the upper or lower respiratory tract. In one aspect, the respiratory disease or disorder in the method can comprise, or alternatively consist essentially of, or yet further consist of asthma, allergic asthma, bronchiolitis or pleuritis. In one aspect, the respiratory disease or disorder in the method can comprise, or alternatively consist essentially of, or yet further consist of: Airway Obstruction, Apnea, Asbestosis, Atelectasis, Berylliosis, Bronchiectasis, Bronchiolitis, Bronchiolitis Obliterans Organizing Pneumonia, Bronchitis, Bronchopulmonary Dysplasia, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Rhinoscleroma, Scimitar Syndrome, Severe Acute Respiratory Syndrome, Silicosis, Tracheal Stenosis, eosinophilic pleural effusions, Histiocytosis; chronic eosinophilic pneumonia; hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease (COPD); Interstitial Lung Disease; allergic rhinoconjunctivitis; allergic conjunctivitis and Topical eosinophilia. In another aspect, the allergic reaction in the method can further comprise, or alternatively consist essentially of, or yet further consist of, hypersensitivity, inflammatory response or inflammation that affects the gastrointestinal tract, the skin or the eye.

In one aspect, the method further comprises, or alternatively consists essentially of, or yet further consists of administering an effective amount of a second drug. In a particular aspect, the second drug in the method further comprises, or alternatively consists essentially of, or yet further consists of an anti-inflammatory, anti-asthmatic or anti-allergy drug. In one aspect, the second drug in the method further comprises, or alternatively consists essentially of, or yet further consists of a hormone, a steroid, an anti-histamine, anti-leukotriene, anti-IgE, anti-α4 integrin, anti-β2 integrin, anti-CCR3 antagonist, β2 agonist or anti-selectin. In one aspect, the method further comprises, or alternatively consists essentially of, or yet further consists of administering the therapy or the compound via ingestion, via inhalation, topically, or a combination thereof. In another aspect, the method comprises, or alternatively consists essentially of, or yet further consists of administering to the subject the compound one, two, three, four or more times daily, weekly, monthly, bi-monthly, or annually. In one aspect, the effective amount of the therapy or the compound in the method further comprises, or alternatively consists essentially of, or yet further consists of from about 0.00001 mg/kg to about 10,000 mg/kg, from about 0.0001 mg/kg to about 1000 mg/kg, from about 0.001 mg/kg to about 100 mg/kg, from about 0.01 mg/kg to about 10 mg/kg, or from about 0.1 mg/kg, about 1 mg/kg body weight. In another aspect, the method comprises, or alternatively consists essentially of, or yet further consists of administering the therapy to the subject substantially contemporaneously with, or within about 1-60 minutes, hours, or days of the onset of an adverse symptom associated with the food allergy, allergic reaction or hypersensitivity. In one aspect, the method can further comprise, or alternatively consist essentially of, or yet further consist of identifying an agent for use in the method, comprising, or alternatively consisting essentially of, or yet further consisting of: a) contacting an HRF-reactive Ig with a test agent in the presence of an HRF monomer, an HRF dimer, or an HRF multimer; and b) determining if the agent inhibits or reduces binding of the HRF-reactive Ig to the HRF monomer, the HRF dimer, or the HRF multimer, wherein a reduction or inhibition of binding identifies the agent as an agent that reduces or inhibits a food allergy, allergic reaction, hypersensitivity, inflammatory response or inflammation.

### EXAMPLES

These examples are provided for illustrative purposes only and not to limit the scope of the claims provided herein.

### Example 1

### Egg Allergy Patients and OIT

Patients (UMIN Clinical Trials Registry UMIN000003943, 43 patients, 37 males and 6 females, average 7.3±2.1 years) were confirmed to have hen's egg allergy by oral challenge test at the enrollment and received rush OIT that enabled 38 participants to eat about one egg (60 g) and 1 g of egg white powder per day without unbearable adverse effects such as refractory gastroenteritis and/or severe anaphylaxis (28). These patients were kept eating this amount of egg for 12 months. Then, after two weeks of egg avoidance, the patients took an oral challenge test, results of which were used to stratify patients into four groups, one without no (threshold, ≥1000 mg egg protein), mild (300-1000 mg), considerable (100-300 mg), and severe (<100 mg) reductions in threshold. HRF, HRF-reactive IgE and HRF-reactive IgG levels in plasma collected before, one week, and 12 months after OIT initiation were measured by ELISA. Due to sample shortage, some samples from one patient was not available. Therefore, 42 initial samples and 37 time-course samples were analyzed.

### Mice

BALB/c mice were bred in the animal facility of RIKEN IMS. Frozen zygotes of FceRIa^{-/-} mice were donated by Takashi Saito (RIKEN IMS) and bred at RIKEN IMS. FcgRIIB^{-/-} mice provided by Toshiyuki Takai (Tohoku University) were also bred at RIKEN IMS. Food allergy experiments with Enpp3^{-/-} mice were conducted at Osaka University. All genetically modified mice were backcrossed to BALB/c background for more than ten generations.

### HRF Inhibitors

GST and GST-N19 were purified using glutathione-agarose (Sigma-Aldrich). HRF-2CA-His₆ expressed by pET-24a(+) plasmid was purified using ProBond™ resin (Invitrogen). All recombinants were further purified by Sephacryl S-100 and dialyzed against PBS. N19 peptide was synthesized by Eurofins Genomics, Tokyo, Japan.

### IgE-Mediated Experimental Food Allergy and OIT

Mice were i.p. sensitized with OVA (50 μg/mouse) plus alum on days 0 and 14. From day 28, mice were i.g. challenged with OVA (25 or 50 mg) or PBS (control) three times a week. Before each challenge, mice were starved for 3 h, then i.g. pretreated with GST, GST-N19 (100 μg/mouse), synthetic N19 peptide, or HRF-2CA. The development of diarrhea was monitored for 90 min after OVA challenge. Diarrhea severity (30) was based on states of stool: solid (score 0), funicular (score 1), slurry (score 2), and watery (score 3).

In a classical tolerance model, five daily oral administrations of 1 mg OVA from day −11 to day −7 were performed before i.p. OVA/alum-sensitization on days 0 and 7 and followed by ig. OVA (30 mg) gavages on days 14, 21, 28 and 35. In another OIT model, mice were i.d. sensitized with OVA (750 μg) on days 0, 13 and 27 and followed by 9 oral OVA (60 mg) challenges from day 44 to day 60 to induce diarrhea in all mice. Then mice were i.g. treated with hourly increasing amounts of OVA on day 100 (0.1, 0.2, 0.5 mg), on day 101 (1, 2, 5 mg) and on day 102 (10, 20, 60 mg).

### Measurement of Parameters of Food Allergy

For histologic analysis, jejunal tissue was fixed in 10% formalin. Mast cells in jejunal sections were detected by chloroacetate esterase staining. IgE, IgG, IgG1, IgG2a, mMCP-1, HRF, HRF-reactive IgE, and HRF-reactive IgG 24 h after the last OVA challenge were measured by ELISA (see Supplemental Information). Cytokine mRNAs in jejunum were quantified by qRT-PCR.

### Preparation of Lamina Propria Cells

Twenty-four hours after the last OVA challenge, mice were sacrificed and small intestines (0.5-15 cm from stomach) were collected. The intestinal tissues were opened longitudinally and washed with HBSS. The washed tissues were incubated with 5 mM EDTA-HBSS for 30 min at 4° C., transferred to 30 ml HBSS in 50 ml tubes, and vigorously shaken to remove intestinal epithelial cells. The tissues were then washed with 10 ml of 10% FCS RPMI medium. Tissues were cut into tiny pieces and incubated with 2.4 mg/ml Collagenase D and 0.2 mg/ml DNase for 30 min at 37° C. Then cells were washed, resuspended in 44% Percoll solution, and slowly loaded on the top of 67% Percoll solution. The cells were centrifuged (1800 rpm, 20 min) and lymphocyte fractions were collected and used for detection of MMC9s (Lin⁻c-Kit⁺ST2⁺IL-17RB⁻Integrin β7^{lo}).

### Flow Cytometry to Quantify MMC9s

The cells were first incubated with 2.4G2 for 15 min at 4° C. and further incubated with Abs (PE-anti-07 integrin mAb (clone FIB504), PerCP/Cy5.5-anti-CD4 mAb (clone RM4-5), PerCP/Cy5.5-anti-CD8 mAb (clone 53-6.7), PerCP/Cy5.5-anti-CD11b mAb (clone M1/70), PerCP/Cy5.5-anti-CD11c mAb (clone N418), PerCP/Cy5.5-anti-B220 mAb (RA3-6B2), PerCP/Cy5.5-anti-Ly-6G/Ly-6C mAb (clone RB6-8C5), PerCP/Cy5.5-anti-CD335 mAb (clone 29A1.4), AlexaFluor647-anti-IL-17RB (clone 9B10), APC/Cy7-anti-CD117 (clone 2B8), Biotin-anti-ST2 (clone DIH9)) (Biolegend) for 30 min at 4° C. After washed, the cells were incubated with SA-BV421 (Biolegend) for 30 min at 4° C. MMC9 population was identified using FACS Canto. Data were analyzed using FlowJo software.

### Ex Vivo Mast Cell Activation Test (MCAT)

Twenty-four hours after the last OVA challenge, mice were sacrificed and small intestines (0.5-15 cm from stomach) were collected. The intestinal tissues were opened longitudinally and washed with PBS. Then, the tissues were incubated with 10 mM EDTA-PBS for 30 min at 37° C. After incubation, the tissues were transferred to 30 ml PBS in 50 ml tubes and vigorously shaken. After washed, the cells were resuspended in 40% Percoll solution and slowly loaded on the top of 75% Percoll solution. The cells were then centrifuged (2000 rpm, 20 min) and lymphocytes were collected. The cells were once centrifuged and resuspended in Tyrode's buffer. Abs (Biotin-anti-CD4 mAb (clone RM4-5), Biotin-anti-CD8 mAb (clone 53-6.7), Biotin-anti-B220 mAb (clone RA3-6B2), Biotin-anti-CD326 mAb (clone G8.8), PE-anti-CD117 (clone 2B8), AlexaFluor647-anti-LAMP-1 mAb (clone 1D4B)) (Biolegend) were added and cells were then stimulated with OVA, anti-IgE (clone RME-1, BioLegend) and recombinant dimeric murine HRF for 15 min at 37° C. After activation, cells were washed and incubated with FITC-streptavidin (Catalog #405202, Biolegend) for 30 min at 4° C. LAMP-1 expression was detected using FACS Calibur. Data were analyzed using FlowJo software.

### ELISA to Quantify HRF Dimer/Multimers

Anti-HRF mAb (clone 2A3, Abnova) was immobilized in a 96-well plate (Corning) at 4° C. overnight. The plate was blocked with 5% skim milk and 10% FCS. After washes, plates were incubated with 50 μl/well of a standard or sample at room temperature (RT) for 2 h on a rotating table. After washes, 1 h incubation with 50 μl of 1 μg/ml biotinylated mAb 2A3 was followed. Then, the plates were incubated with 50 μl of 1:1000 diluted avidin-HRP (BioLegend) at RT for 30 min. After extensive washings, add 50 μl/well of ECL Pro (PerkinElmer) to the plate and luminescence was measured in a luminescent microplate reader.

### Western Blot Analysis of HRF in Various Cells

The following cell lines were cultured in DMEM plus 10% FCS except for U-937 cells which were cultured in RPMI1640 plus 10% FCS: CMT-93 mouse and Caco-2 (with 20% FCS) human intestinal epithelial cell lines; NIH/3T3 mouse fibroblast line; Raw 264.7 mouse and U-937 human macrophage lines; BEAS-2B human bronchial epithelial cell line; 293T human embryonic kidney cell line (obtained from ATCC, Manassas, Va., except for NIH/3T3, which was provided by S. A. Aaronson, NIH). Fibroblast-like stromal cells were cultured from small intestines of BALB/c mice in DMEM/10% FBS. CD4⁺ T and B cells were purified from mouse spleens using BD IMag™ Mouse CD4 and B cells enrichment set-DM. Low-density bone-marrow cells were cultured in the presence of 100 ng/ml of stem cell factor (PeproTech) and 100 ng/ml of FLT-3 ligand (R&D Systems) for 4 days, and with 10 ng/mL of IL-5 (R&D Systems) thereafter for 10 days to obtain bone marrow-derived eosinophils (44). Bone marrow cells were cultured in the presence of 20 ng/ml of GM-CSF for 6 days and adherent cells were collected as bone marrow-derived macrophages. Non-adherent cells were further cultured with 5 ng/ml GM-CSF and the non-adherent cells were collected as bone marrow-derived dendritic cells. Bone marrow cells were cultured in IL-3-containing medium for 4-6 weeks to generate >95% c-Kit⁺FcεRI⁺ mast cells. All cells were stimulated with 100 ng/ml LPS, 20 ng/ml PMA plus 1 mM Ionomycin and 100 ng/ml IL-13 in 0.1% BSA RPMI1640 medium for 3 h or 6 h.

### Immunofluorescence Microscopy

Jejunum biopsy was fixed in 4% paraformaldehyde at 4° C., washed in 10-20% sucrose in phosphate buffered saline, and embedded in O.C.T. compound (Sakura Finetek, Japan). Cyrosections were made in 6 μm and blocked with 2% skim milk. Immunostaining was performed with anti-HRF (rabbit polyclonal FL-172), anti-GST (rabbit polyclonal Z-5) (Santa Cruz Biotechnology), anti-IgE (clone RME-1 from Biolegend and clone R35-72 from BD Biosciences), anti-mMCP1 (Catalog #AF5146, R&D Systems), anti-CD45 (clone 30-F11), anti-CD63 (clone NVG-2) (Biolegend), and anti-Siglec F (clone E50-2440, BD Biosciences) as primary antibodies, and anti-Rabbit Alexa Fluor 647 (Catalog #A21245), anti-Rat Alexa Fluor 568 (Catalog #A11077), and anti-Sheep Alexa Fluor 555 (Catalog #A11015) (Thermo Fisher Scientific) as secondary antibodies. Coverslips were mounted with Prolong Gold with DAPI (Thermo Fisher Scientific) and fluorescence was observed under Leica TCS SP2 confocal microscopy (Leica) and Nuance Multi spectral Imaging System (PerkinElmer).

### Statistics

Statistical analysis was performed using Prism6 software (GraphPad, San Diego, Calif.). Data are presented as mean±SEM in all figure parts in which error bars are shown. Groups were analyzed by two-tailed Student's t-test, paired t-test and two-way ANOVA, unless otherwise indicated. P values of <0.05 were considered statistically significant.

### Study Approval

Human study was performed according to The Declaration of Helsinki Principles. The study protocol was approved by the Ethical Review Board of Mie National Hospital (Mie, Japan) and RIKEN Yokohama Campus Ethics Committee (Yokohama, Japan). Written informed consent was obtained from each of the study participants and their guardians prior to participation.

Animal experiments were approved by the Animal Care and Use Committee of the La Jolla Institute for Allergy and Immunology (La Jolla, Calif.) and the RIKEN Yokohama Animal Experiments Committee (Yokohama, Japan), and Animal Experimentation Committee of Osaka University (Osaka, Japan).

### Enzyme-Linked Immunosolvent Assays (ELISAs)

ELISA kits for human total IgE, IgG and mMCP-1 were purchased from eBioscience. Mouse total IgE, IgG1 and IgG2b (not a) levels were similarly analyzed using the following antibodies to capture and to detect the antibodies: purified rat anti-mouse IgE (BD Biosciences, Cat 553413) and biotin-conjugated rat anti-mouse IgE (BD Biosciences, Cat 553419); purified rat anti-mouse IgG1 (BD Biosciences, Cat 553445) and biotin-conjugated rat anti-mouse IgG1 (BD Biosciences, Cat 553441); purified rat anti-mouse IgG2b (BD Biosciences, Cat 553396) and biotin-conjugated rat anti-mouse IgG2b (BD Biosciences, Cat 553393). 96-well ELISA plates were coated overnight with capturing antibodies (each at 1 μg/ml in 0.1 M carbonate buffer [pH 9.5]). The plates were washed and blocked with 10% FCS. Next, diluted plasma or sera were incubated in the coated plates, after which bound immunoglobulins were detected by incubation with biotinylated detection antibodies followed with HRP-conjugated streptavidin (BD Biosciences). Color was developed using TMB substrate (Biolegend), and absorbance at 450 nm was measured and corrected with absorbance at 570 nm.

HRF was measured using anti-TPT1/TCTP antibody (Novus Biologicals, Cat #H00007178-M06, clone 2A3) for capturing and anti-TPT1/TCTP antibody (self-biotinylated mAb, Novus Biologicals, Cat #H00007178-M03, clone 2C4) and streptavidin-β-Gal conjugate (Roche, Cat #11112481001) for detection. After incubation with streptavidin-β-Gal conjugate and washing, ELISA wells were incubated with 0.2 mM 4-Methylumbelliferyl-β-D-galactopyranoside (4-MU-Gal, Sigma-Aldrich, Cat #M1633) for 1 h at 37° C. Fluorescence was measured at excitation of 365 nm and emission of 445 nm. HRF-reactive IgE was measured using in-house ELISAs: ELISA wells were coated with 10 μg/ml recombinant human (or mouse) HRF-His6 in 0.1 M sodium carbonate buffer (pH 9.5) for overnight at room temperature (RT). After washings, the wells were blocked with ImmunoBlock (DS Pharma Biomedical, Japan, Cat #CTKN001) for 2 h. The wells were washed, and incubated with 1 μg/ml biotin anti-human IgE (anti-mouse IgE) for 1 h at RT. Then the wells were washed and incubated with streptavidin-β-Gal conjugate, followed washings and incubation with 0.2 mM 4-MU-Gal for 1 or 2 h. Fluorescence was measured at excitation of 365 nm and emission of 445 nm. HRF-reactive IgG was similarly analyzed except for the use of biotin anti-human IgGs or biotin anti-mouse IgGs instead of anti-human IgE. Biotin anti-human IgE, IgG1 and IgG4 antibodies were also purchased from BD Biosciences for HRF-reactive Ig ELISA (Cat 555858, 555869 and 555882, respectively).

### HRF-Reactive IgE Increase Index

HRF-reactive IgE increase ratio was calculated by the formula: (HRF-reactive IgE at 12 months—HRF-reactive IgE at 1 week) divided by (HRF-reactive IgE before OIT). Increase index was further calculated by the formula: 2 divided by {1+e^{-(HRF-reactive IgE increase ratio)}}−1. This index was compared between patients with no decrease in threshold and patients with severe decrease in threshold (FIG. 61I).

### HRF Promotes Intestinal Inflammation During the Elicitation Phase of Murine Food Allergy

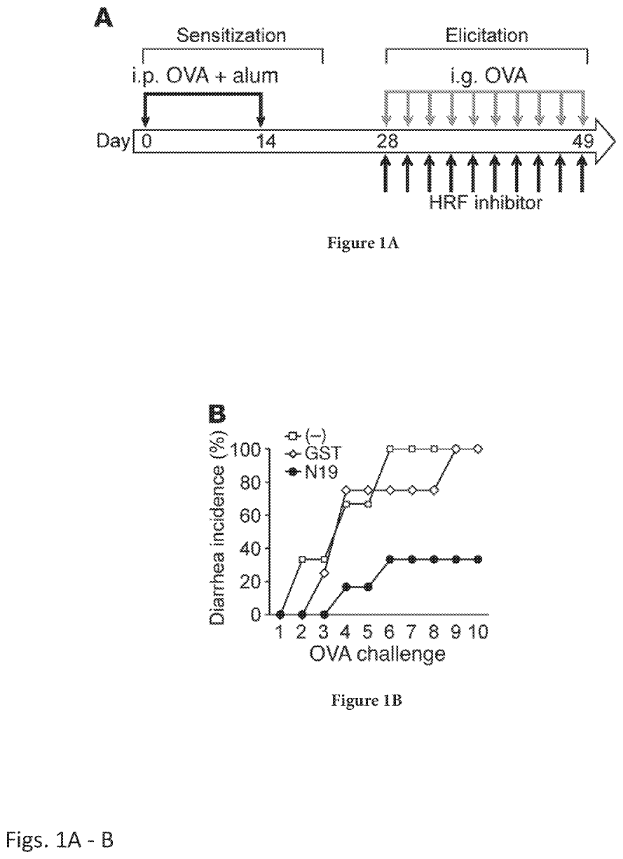

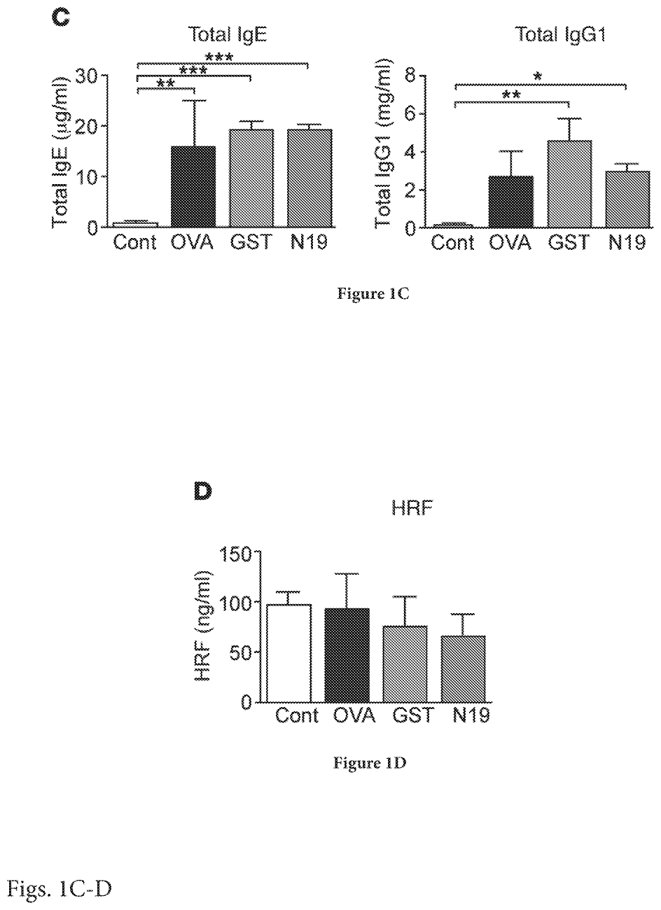

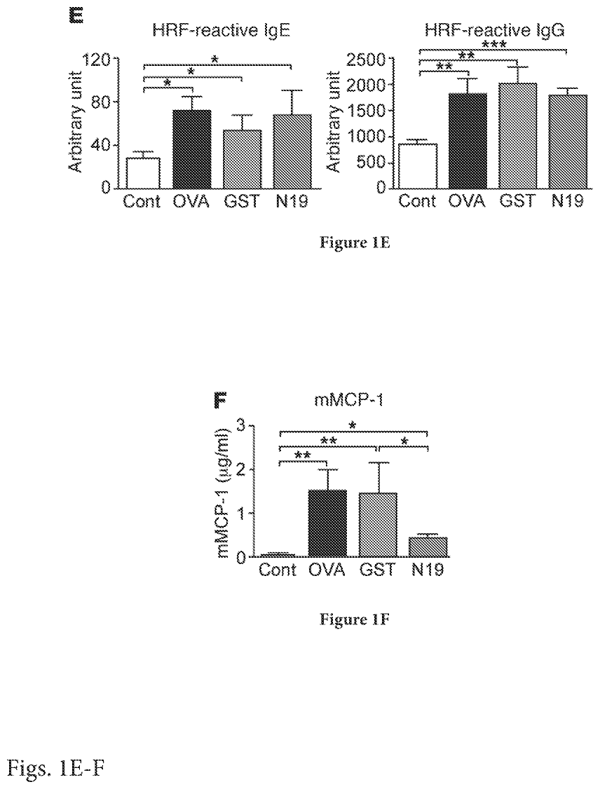

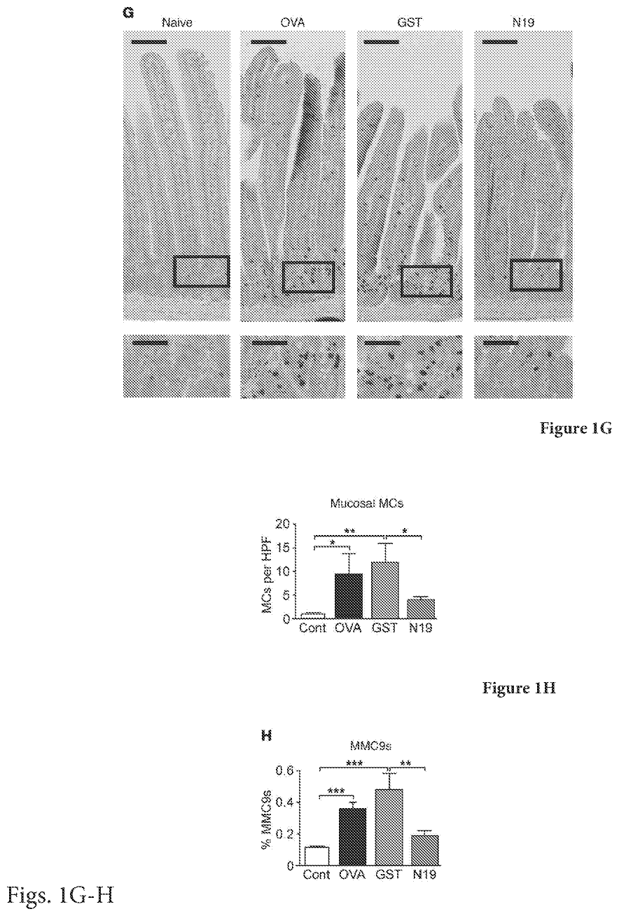

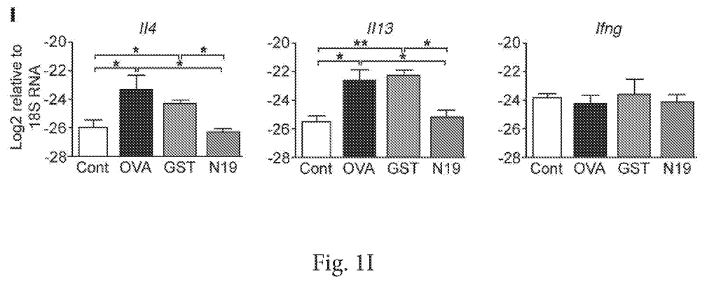

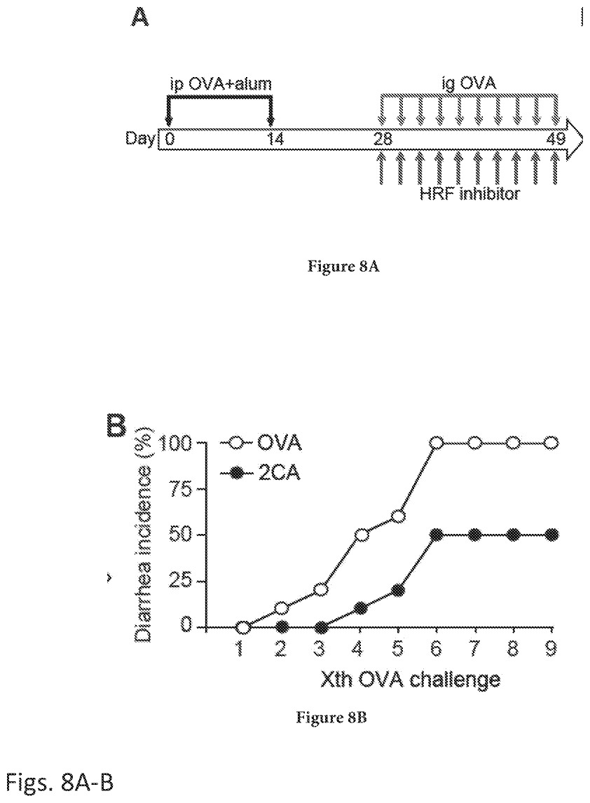

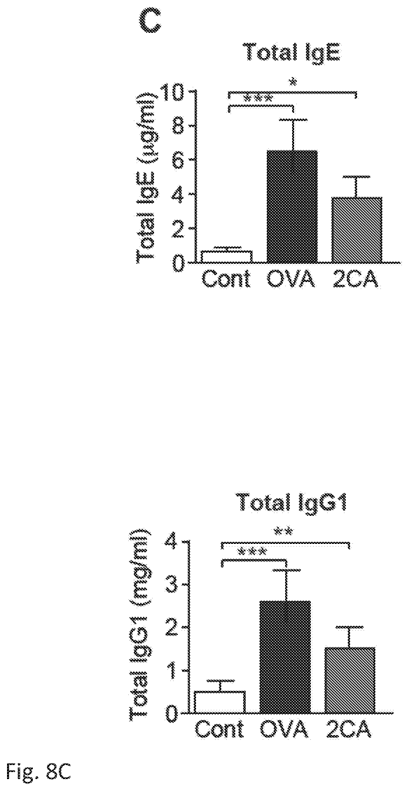

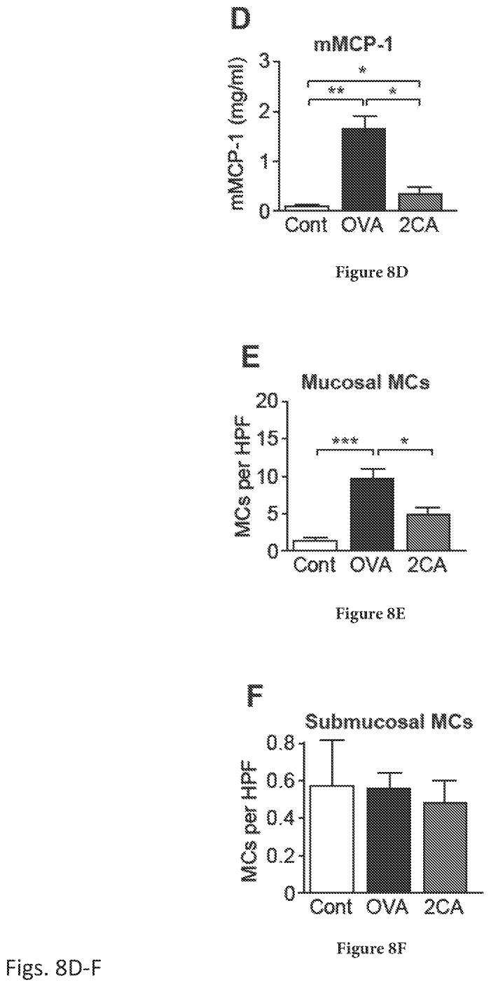

Mice immunized intraperitoneally (i.p.) with OVA in the presence of alum were repeatedly challenged with i.g. gavages of OVA, leading to the development of diarrhea and type 2 inflammation in the small intestine (9, 10). Initial diarrheal events occurred mostly from the 2′ to the 6^{th} challenge (FIG. 1A, FIG. 1B). As shown previously (9), serum levels of total IgE and IgG1 as well as OVA-specific IgE and IgG were increased in diarrheal mice, compared with non-sensitized, OVA-challenged control mice (FIG. 1C and data not shown). Interestingly, serum levels of HRF-reactive IgG and HRF-reactive IgE, but not HRF, were also increased (FIG. 1D, FIG. 1E). When sensitized mice were pretreated i.g. with an HRF inhibitor, GST-N19, before each OVA challenge, the onset of diarrhea was delayed and its incidence was reduced (FIG. 1A, FIG. 1B). Consistent with the established role of mast cells in this model (9), the increased serum level of the mast cell protease mMCP-1 was sustained for >8 h after OVA challenges and reduced by GST-N19 pretreatment (FIG. 1F and data not shown). Furthermore, HRF inhibitor reduced the number of mucosal mast cells found in jejunum (FIG. 1G). Flow cytometry confirmed increased IL-9-producing mucosal mast cells (MMC9s), which are precursors to granular mucosal mast cells and critical in this model (20), in food allergic mice, and HRF inhibitor reduced MMC9s in jejunum (FIG. 1H). A robust correlation was found between diarrhea occurrence and numbers of mucosal mast cells (Spearman's correlation r=0.82, p<0.0001), but numbers of submucosal mast cells were not increased by OVA challenges (FIG. 8 and data not shown). Transcriptome and qRT-PCR analyses of jejunum (FIG. 1I and data not shown) showed increased Th2 cytokine mRNAs (Il4 and Il13) in diarrheal mice, which was repressed in inhibitor-pretreated mice. By contrast, levels of total IgE, IgG1, IgG2a or HRF-reactive IgE and IgG were not reduced by HRF inhibitor (FIG. 1C, FIG. 1E and data not shown). I.g. administration of a synthetic N19 peptide or HRF-2CA, which is a monomeric mutant of mouse HRF (19), in place of GST-N19 exhibited similar inhibitory effects on diarrhea occurrence, intestinal inflammation, mucosal mast cells and serum mMCP-1 levels, but failed to affect levels of IgE, IgG1, or HRF-reactive IgE and IgG (FIG. 8).

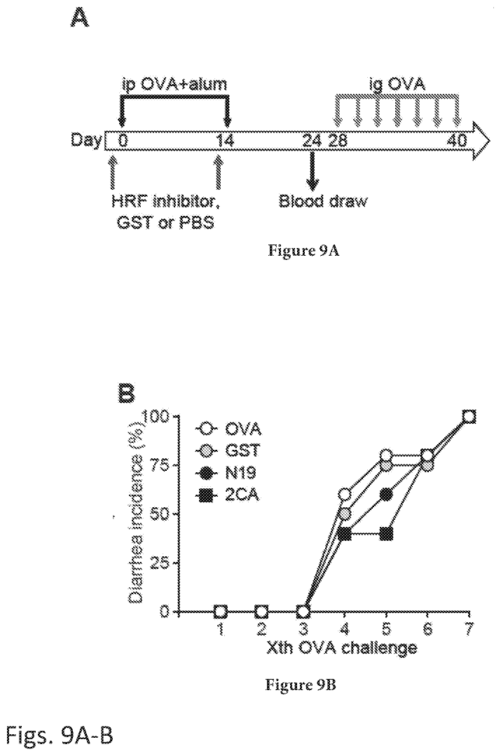

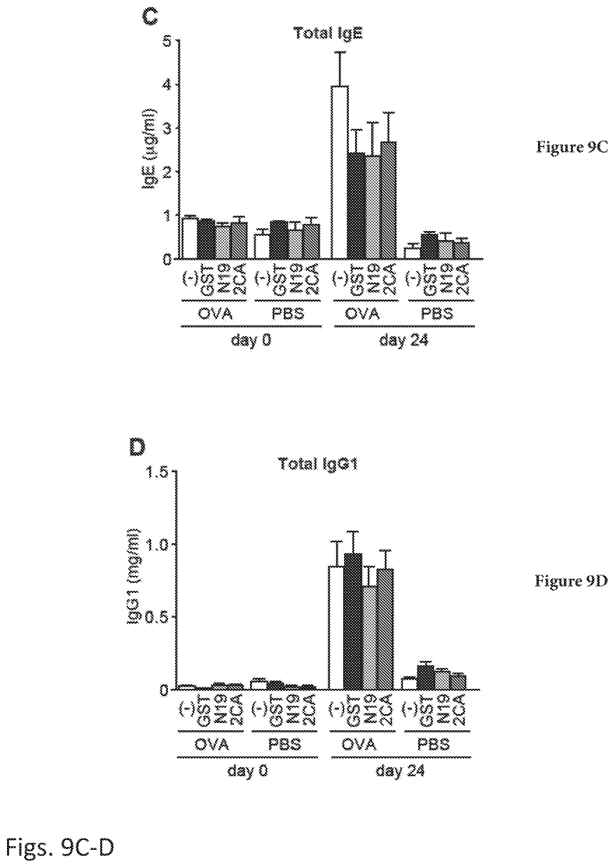

While allergen-induced increases in IgE and IgG levels were not changed in HRF inhibitor-treated vs. -nontreated mice, the efficacy of HRF inhibitors suggested that they suppress the allergen-triggered elicitation phase of food allergy by targeting mast cells. Whether HRF inhibitors affect the sensitization phase of food allergy was tested. Pretreatment with HRF inhibitor before sensitization with OVA plus alum did not affect diarrhea occurrence or levels of IgE or IgG1 (FIG. 9), indicating that HRF regulates the elicitation, but not sensitization, phase in this food allergy model.

### HRF Inhibitors Suppress Allergic Diarrhea Via FcεRI

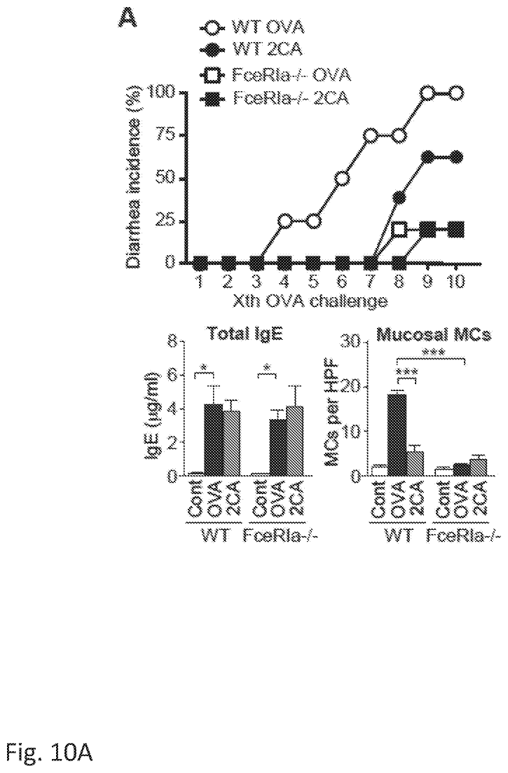

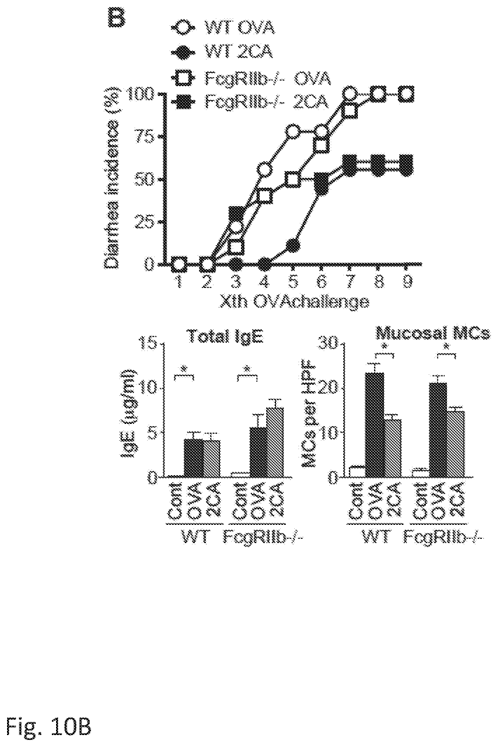

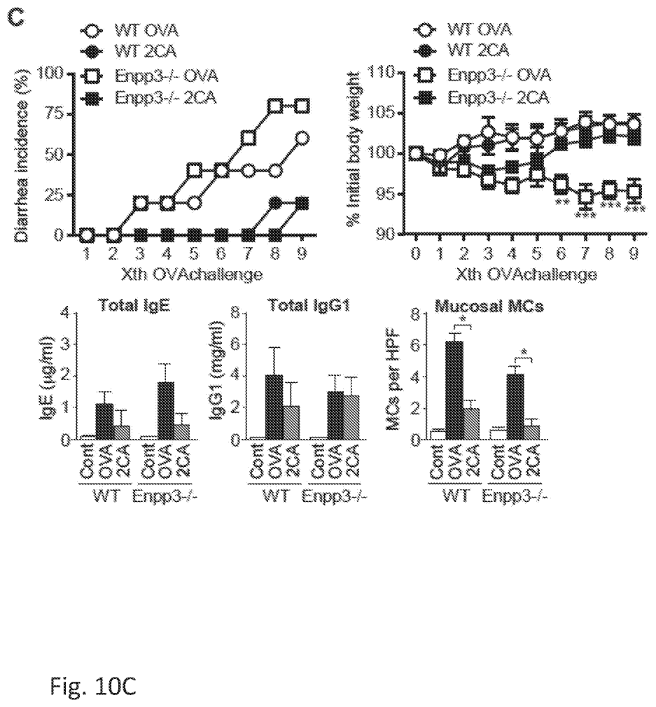

A crucial role for FcεRI in food allergy was previously demonstrated in this and other murine models (9, 21). Applicant confirmed it using FceRIa^{-/-} mice, as allergic diarrhea was severely delayed and suppressed in FceRIa^{-/-} mice. Accordingly, numbers of mucosal mast cells were low and, more importantly, effects of HRF inhibitor were not observed in FceRIa^{-/-} mice (FIG. 10A).

Since the IgG Fc receptor FcεRIIB inhibits FcεRI-mediated mast cell activation (22) and is critical for the efficacy of allergy immunotherapy (23), Applicant also tested the role of this receptor in food allergy using FcgRIIB^{-/-} mice. Allergic diarrhea was seen in FcgRIIB^{-/-} mice similar to WT mice, and HRF inhibitor suppressed allergic diarrhea in the mutant mice (FIG. 10B). Ectonucleotide pyrophosphatase-phosphodiesterase 3 (E-NPP3, also known as CD203c), also negatively regulates FcεRI-induced activation (24).

Indeed, Enpp3^{-/-} mice exhibited enhanced allergic diarrhea accompanied by weight loss, as compared with WT control. Again, HRF inhibitor suppressed allergic diarrhea and weight loss in Enpp3^{-/-} mice (FIG. 10C). These results collectively support the notion that HRF promotes allergic diarrhea and type 2 inflammation via FcεRI.

Mucosal Mast Cells from Allergic Mice are Activated by Allergen and HRF

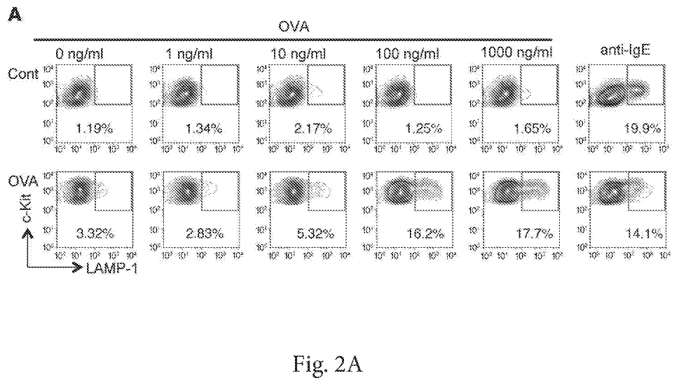

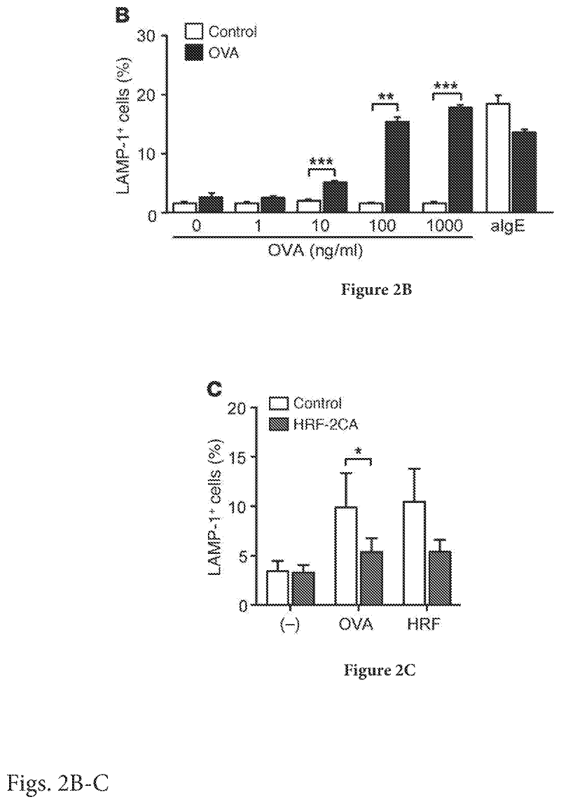

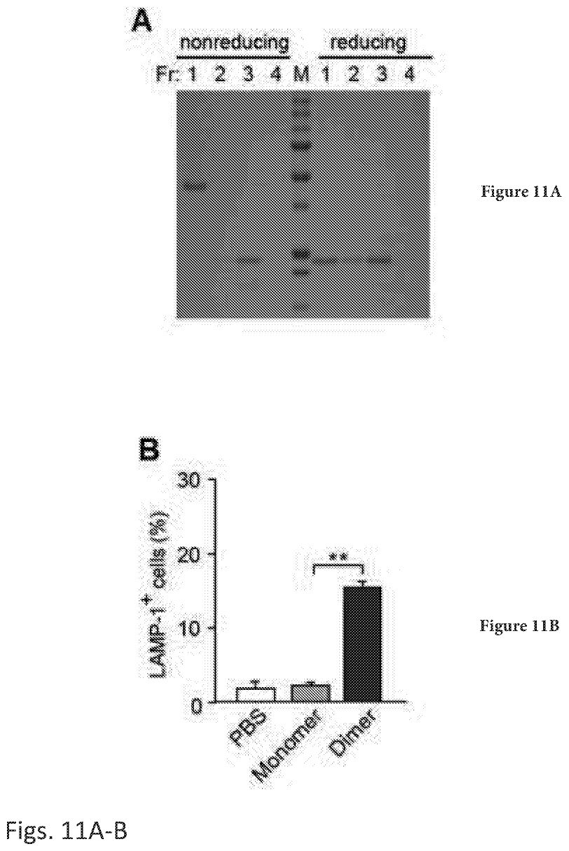

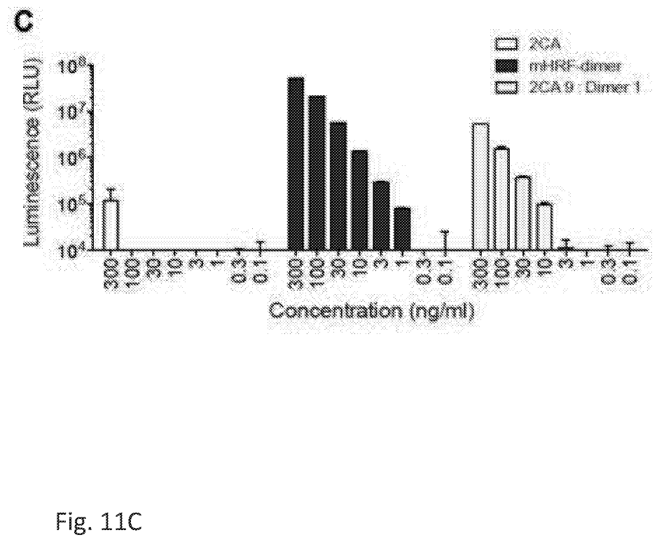

Reactivity of ex vivo mucosal mast cells in the small intestine was investigated in a new assay termed mast cell activation test (MCAT). When cell mixtures containing mucosal mast cells derived from non-sensitized mice were stimulated with OVA, few mast cells were activated (FIG. 2A, FIG. 2B). By contrast, 2.48±0.136% (mean±SEM) of c-Kit⁺ mucosal mast cells derived from allergic mice expressed an activation marker, lysosomal-associated membrane protein 1 (LAMP-1), on their cell surface without stimulation, and they were further activated by OVA in a dose-dependent manner (FIG. 2A, FIG. 2B). Since mucosal mast cells derived from both non-sensitized and allergic mice were similarly activated by anti-IgE antibody, FcεRI molecules in both mice should have been occupied by IgE in vivo. Differential reactivity of the cells to OVA indicated that OVA-specific IgE molecules occupied a substantial proportion of FcεRI on mast cells in allergic mice, whereas FcεRI in non-sensitized mice were occupied by nonspecific IgE. Recombinant dimeric, but not monomeric, HRF activated ex vivo mucosal mast cells (FIG. 11A, FIG. 11B). Moreover, HRF inhibitors (i.e., HRF-2CA and N19 peptide) significantly suppressed HRF-induced as well as OVA-induced activation of mucosal mast cells derived from allergic mice (FIG. 2C and data not shown). These ex vivo observations of OVA-induced mast cell activation likely reflect what happened in the small intestine of food allergic mice.

### HRF Dimer Increases in the Small Intestine of Allergic Mice

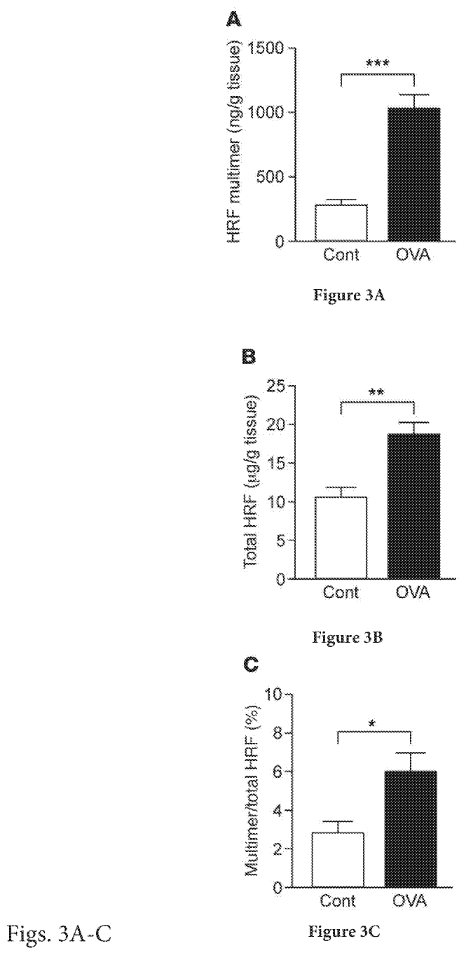

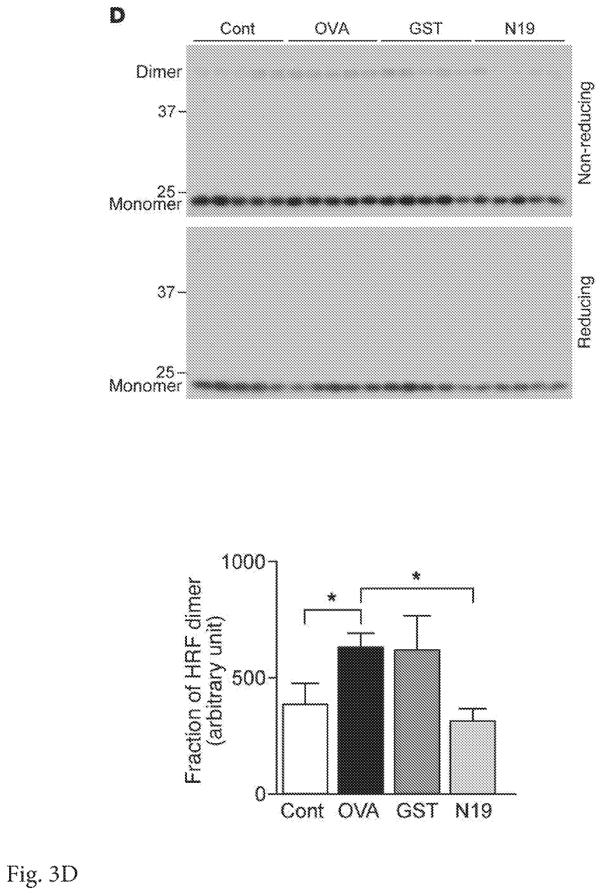

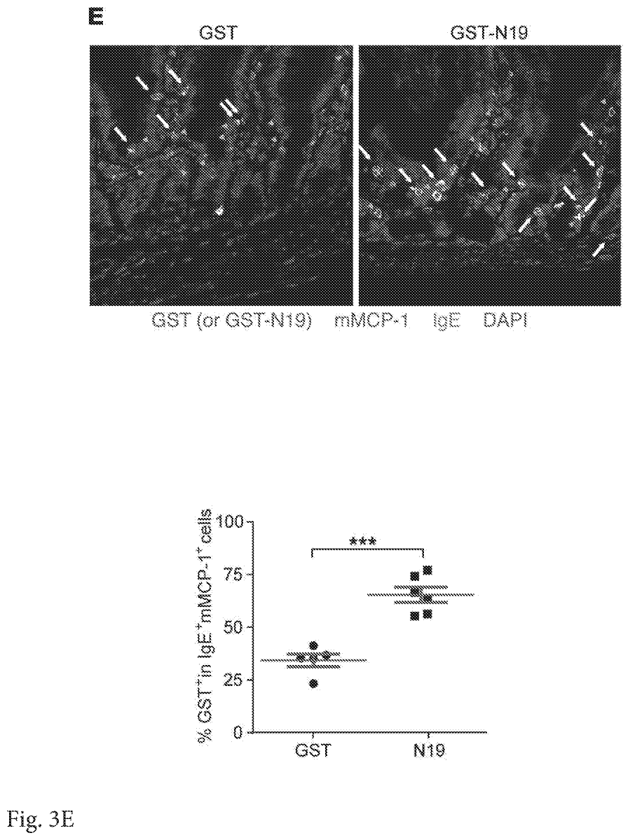

Disulfide-linked HRF dimers bind to certain IgE molecules, leading to cross-linking of IgE-bound FcεRI molecules and their consequent activation (FIG. 11A, FIG. 11B and ref (19)). Applicant thus quantified HRF multimers in inflammatory sites. Jejunal tissues were harvested 24 h after 10^{th} OVA challenge, homogenized, and extracted with PBS for a new ELISA to quantify HRF multimers (FIG. 11C). Three-fold more amounts of HRF multimers were found in allergic than in control mice (FIG. 3A), and the total amount of HRF and the ratio of HRF multimers to total HRF were also increased in allergic mice (FIG. 3B, FIG. 3C). Consistent with this, western blot analysis showed that the small intestine of allergic mice had significantly higher ratios of dimeric to monomeric HRF than those of control mice and the ratios were reduced by HRF inhibitor (FIG. 3D). The increased multimer-to-monomer ratio was likely due to oxidative stress under allergic conditions (25, 26). These results suggest that HRF dimer/multimers promote mast cell activation in the small intestine of allergic mice.

### HRF Inhibitors Target Mast Cells

Applicant next tested whether HRF inhibitors target mucosal mast cells. To this end, mice with allergic diarrhea were i.g. gavaged with GST-N19 or GST, and their location was identified 1 h after their gavage. Confocal microscopy showed that 71.4±0.64% of jejunal IgE⁺ cells were also positive for mMCP-1, while other mast cell markers such as mMCP-4 or mMCP-6 were positive for less than 20% of IgE⁺ cells (data not shown). It was found that about 65% of IgE⁺ mMCP-1⁺ mast cells were co-localized with GST-N19 whereas only 34% of IgE⁺ mMCP-1⁺ mast cells were co-localized with GST (FIG. 3E). Preferential targeting of His-tagged HRF-2CA to IgE⁺ mMCP-1⁺ mast cells was also observed using anti-His tag (64.0±2.69%) (data not shown). These results suggest that HRF inhibitors target mainly mucosal mast cells, although their inhibitory effects on basophils might be contributory.

### Intestinal Epithelial Cells, Fibroblasts and Various Immune Cells Secrete HRF

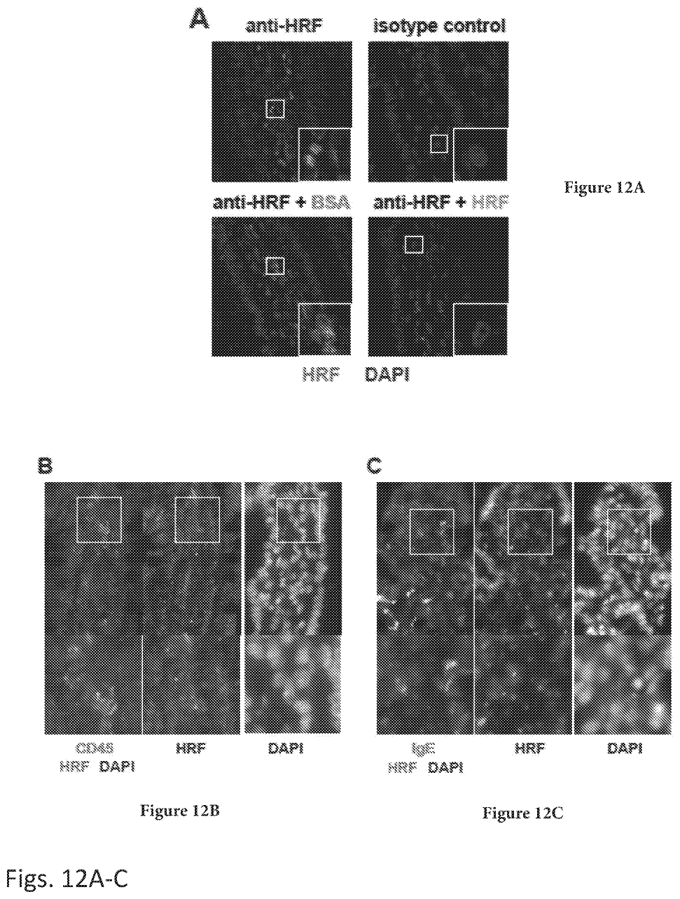

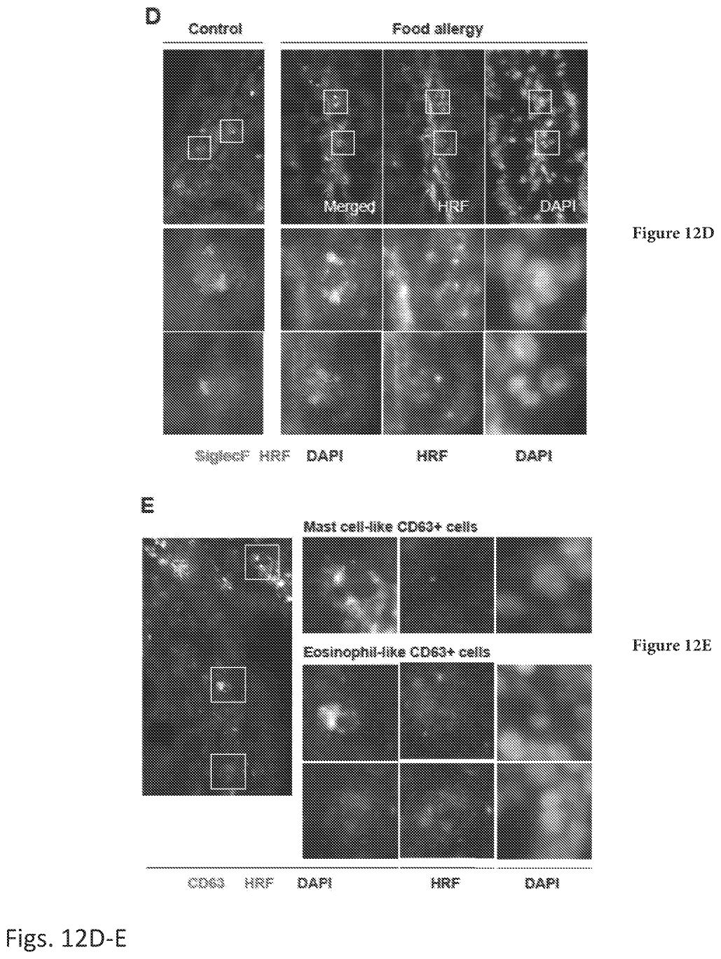

Immunofluorescence microscopy analysis was performed to localize HRF in jejunum, the most proximal intestinal segment where diarrhea began in this model of food allergy. HRF staining was detected both inside and outside of nuclei of intestinal epithelial cells, CD45⁺ hematopoietic cells and CD45⁻ stromal cells in lamina propria (FIG. 12A, FIG. 12B). Among hematopoietic cells, IgE⁺ cells were surrounded by HRF (FIG. 12C), and Siglec F⁺ cells with bi-lobed nuclei were co-stained with HRF (FIG. 12D). Moreover, CD63⁺ cells, most of which were reported to be eosinophils in the steady state lamina propria (27), were also HRF positive (FIG. 12E), suggesting that the eosinophils are HRF producers. Large cells with vesicular CD63 expression appeared after food allergy induction and were negative for intracellular HRF. These may be mast cells. By contrast, CD3⁺ and B220⁺ cells were barely positive for HRF (data not shown).

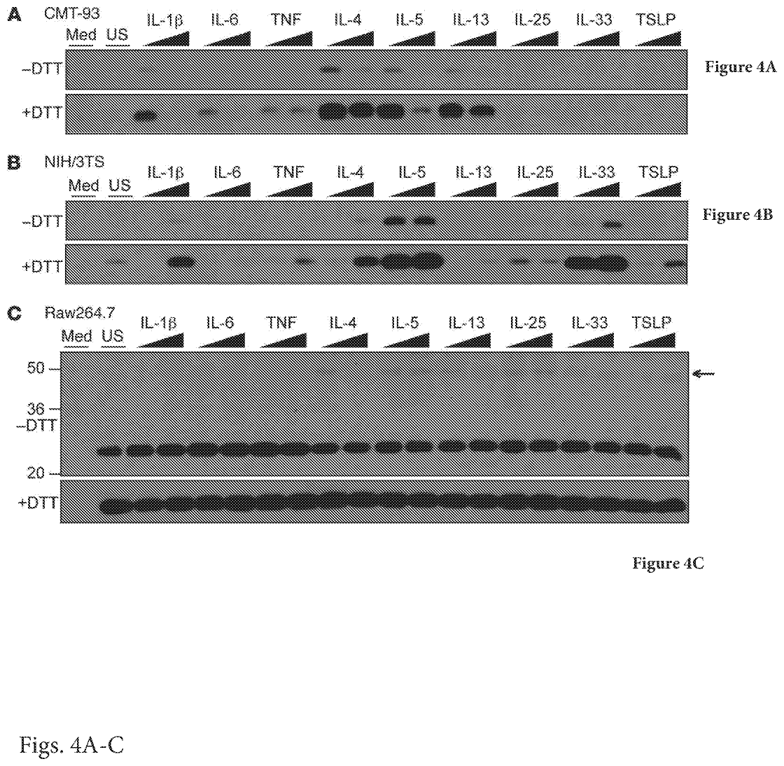

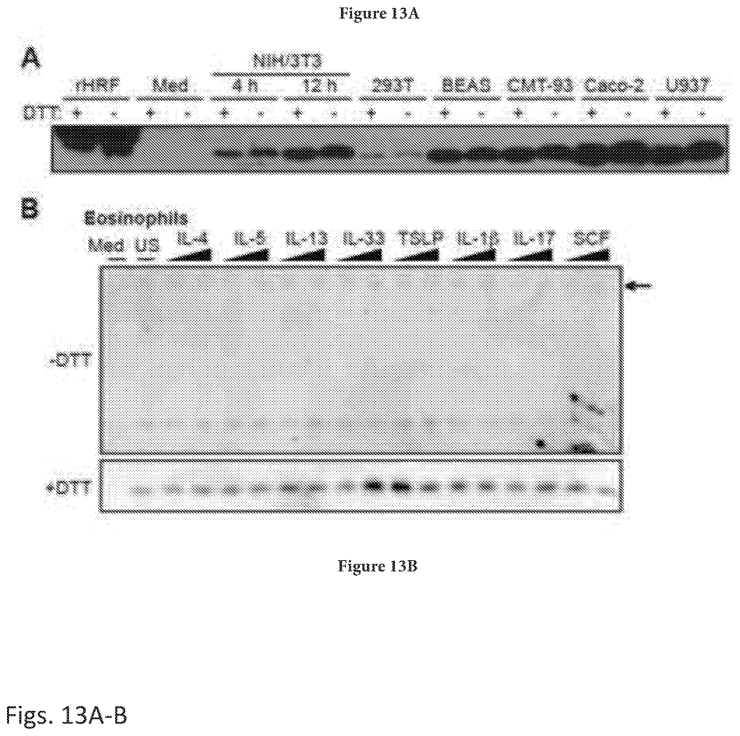

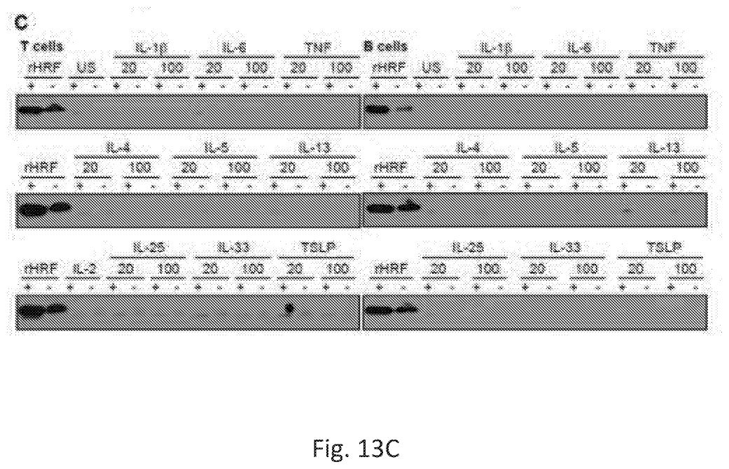

Applicant then investigated which cell types secrete HRF in in vitro cultures. Intestinal epithelial cells, fibroblasts, and various immune cells were tested. CMT-93 murine intestinal epithelial cells constitutively secreted a considerable amount of HRF monomer and stimulation with Th2 cytokines (IL-4, IL-5 and IL-13) and proinflammatory cytokines (IL-1β, IL-6 and TNF) strongly induced HRF secretion (FIG. 4A and FIG. 13A). By contrast, epithelial-derived Th2-promoting cytokines (TSLP, IL-25, and IL-33) failed to induce HRF secretion in CMT-93 cells. Fibroblasts including primary intestinal fibroblasts from newborn mice and NIH/3T3 cells also secreted HRF constitutively (FIG. 4B and data not shown) and stimulation with Th2 cytokines (IL-4 and IL-5), IL-1β, and epithelial-derived cytokines, particularly IL-33, enhanced HRF secretion. IL-9 rather suppressed HRF secretion (data not shown). Consistent with results shown by imaging analysis, bone marrow-derived eosinophils produced both HRF monomer and dimer constitutively (FIG. 13B). Stimulation with IL-33 and TSLP increased HRF multimer in eosinophils. Murine RAW267.4 and human U937 macrophage lines also secreted HRF constitutively (FIG. 4C and FIG. 13A). IL-4, IL-5, IL-13 and IL-25 increased HRF dimer in RAW267.4 cells, while IL-1β, IL-6 and TNF increased the secretion of HRF monomer (FIG. 4C). Both T and B cells secreted modest amounts of HRF, which were not affected significantly by the cytokines (FIG. 13C). By contrast, treatment of dendritic cells, bone-marrow-derived macrophages, and mast cells with various classes of stimulants increased the expression of intracellular HRF but failed to secrete significant amounts of HRF (data not shown). Thus, HRF could be secreted by various cell types present in the small intestine. Taking cell numbers into account, intestinal epithelial cells and fibroblasts seem to be the major cellular sources of secreted HRF, which dramatically increases in response to Th2 and proinflammatory cytokines.

### HRF Inhibitors Ameliorate the Severity of Food Allergy in a Therapeutic Context

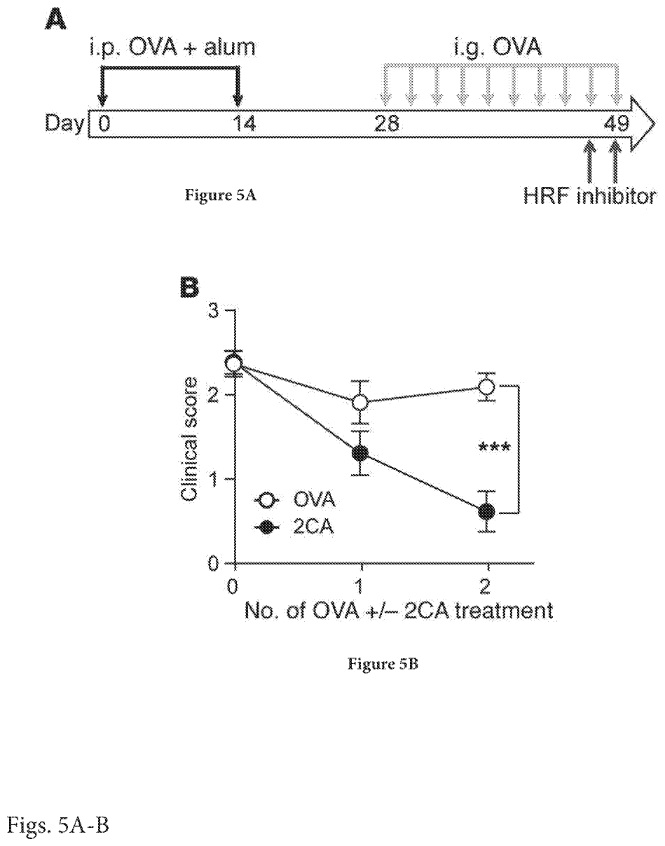

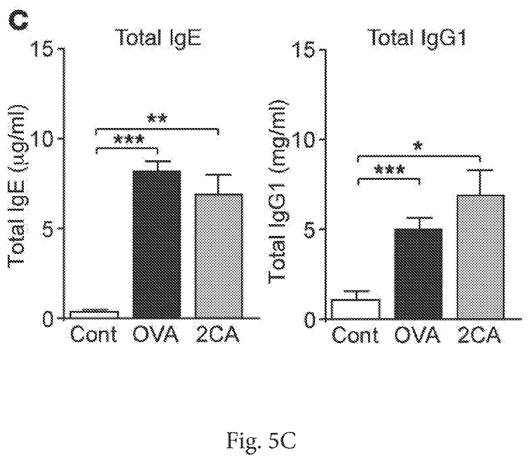

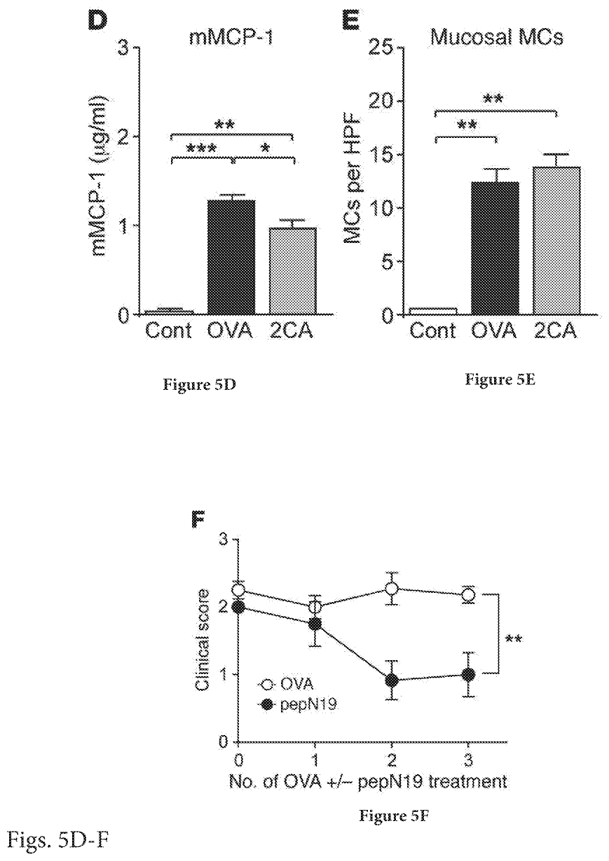

Applicant next tested whether HRF inhibitors modulate food allergy after diarrhea has begun. To this end, mice were divided into two cohorts after OVA-sensitized mice showed diarrhea by the 7th challenge of OVA. One cohort of mice was intravenously (i.v.) treated twice with PBS followed by OVA challenge, and the other were i.v. treated twice with 30 μg of HRF-2CA followed by OVA challenge (FIG. 5A). Clinical scores were significantly reduced by HRF-2CA (FIG. 5B). Therapeutic treatment of HRF inhibitor did not reduce total IgE, IgG1 (FIG. 5C) or mucosal mast cells (FIG. 5E). However, HRF inhibitors modestly but significantly reduced serum mMCP-1 levels (FIG. 5D), suggesting that the activation, but not the growth or differentiation state, of mucosal mast cells can be inhibited by HRF inhibitor after the allergic state is established. Similar therapeutic effects were observed by synthetic N19 peptide (FIG. 5F) or GST-N19 (data not shown). These results collectively demonstrate that therapeutic administration of HRF inhibitors reduces the severity of food allergy.

### HRF-Reactive Immunoglobulins During OIT in Human Food Allergy Patients

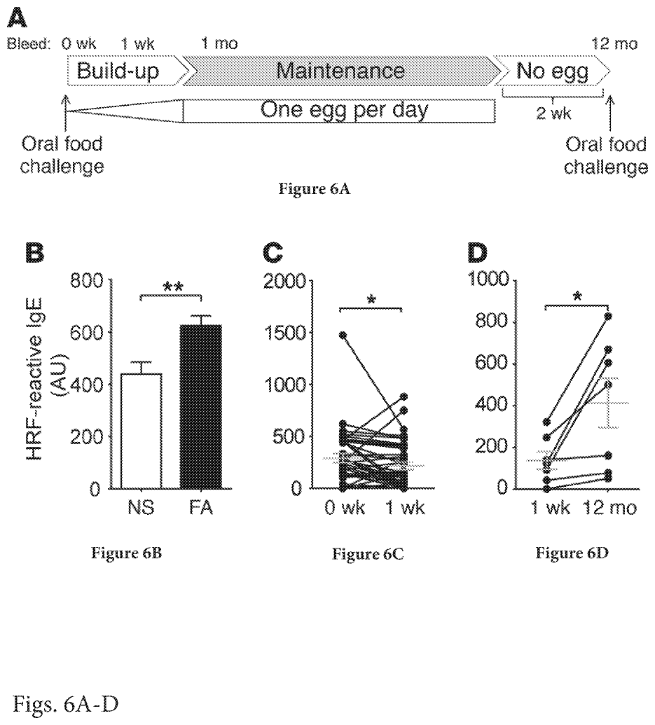

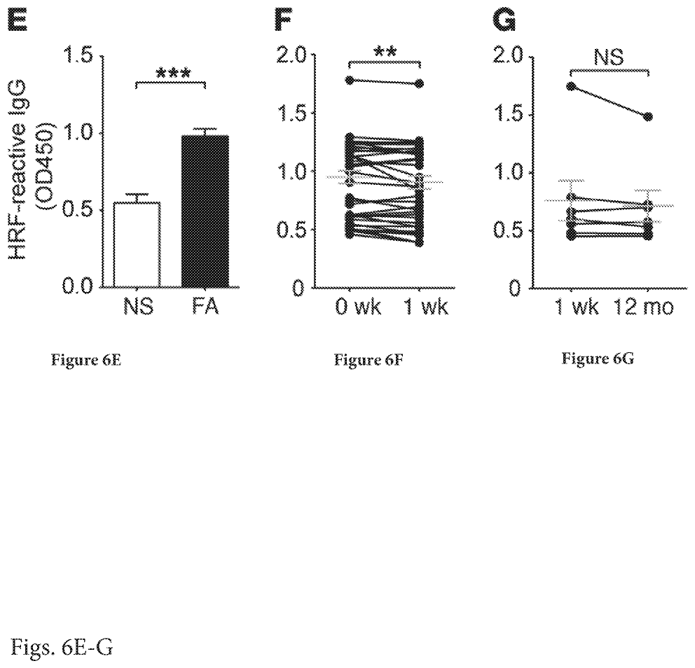

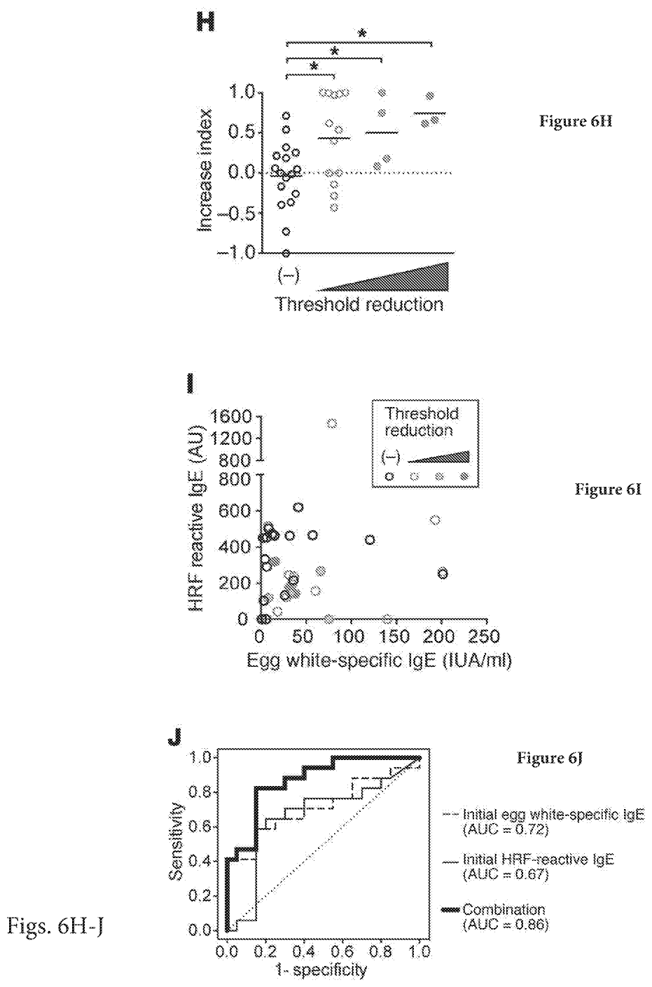

Applicant next investigated whether HRF is involved in human food allergy. Similar to mouse food allergy results, egg allergy patients had plasma levels of HRF similar to normal individuals (data not shown) and significantly higher levels of HRF-reactive IgE and IgG levels (FIG. 6B, FIG. 6E). Full time-course samples from patients who were treated with rush OIT (28) to attain a level of desensitization without showing unbearable adverse effects such as refractory gastroenteritis and/or severe anaphylaxis (FIG. 6A) were analyzed. Plasma levels of HRF were not changed during the OIT (data not shown). However, levels of HRF-reactive IgE/IgGs/IgG1/IgG4 were modestly reduced one week after the initiation of OIT (FIG. 6C, FIG. 6F and data not shown). Following two weeks of allergen avoidance after the end of the 12 month-maintenance dosing, patients exhibited a wide range of sensitivity to egg protein upon oral challenge. Interestingly, HRF-reactive IgE levels in the patients who exhibited a low threshold (sensitive to ≤300 mg of egg white protein) were significantly higher than their HRF-reactive IgE levels one week after the OIT initiation (FIG. 6D, FIG. 6H), whereas HRF-reactive IgE levels in those who exhibited higher thresholds (>300 mg of egg white protein) were not significantly different from their one-week values (data not shown). By contrast, HRF-reactive IgGs/IgG1/IgG4 levels after two weeks of allergen avoidance following the 12-month maintenance period were similar to the levels one week after the OIT initiation in all patients, regardless of their changes in threshold to egg protein (FIG. 6G and data not shown).

Consistent with earlier studies suggesting that the initial antigen-specific IgE level is a good predictive biomarker for the prognosis of OIT (29), low egg white-specific IgE level before OIT was predictive for sustained desensitization to egg (FIG. 6J). In addition, all patients with HRF-reactive IgE above a certain level (400 counts) before OIT initiation tolerated at least 1,000 mg of egg protein after 12 months of OIT (FIG. 6I). The HRF-reactive IgE level, which showed no correlation to egg white-specific IgE (Pearson's correlation r=0.12, p=0.47) or to total IgE (r=0.0071, p=0.97) (FIG. 6I and data not shown), was not only predictive for good prognosis by itself, but also synergistically predictive when combined with egg white-specific IgE, reaching a specificity of 82% and sensitivity of 85% (FIG. 6J). These results suggest that HRF-reactive IgE might serve as an independent predictive biomarker for OIT outcome.

### HRF-Reactive IgE Levels are Sustained Low in Murine OIT Models

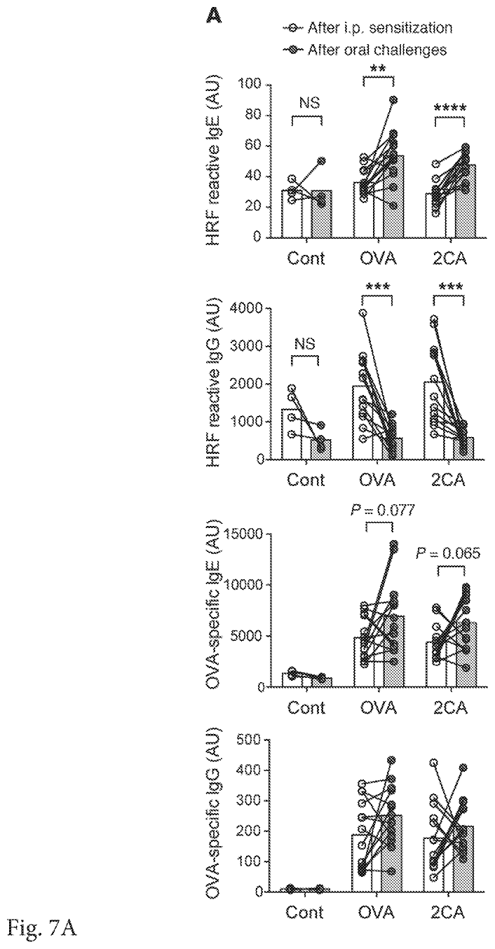

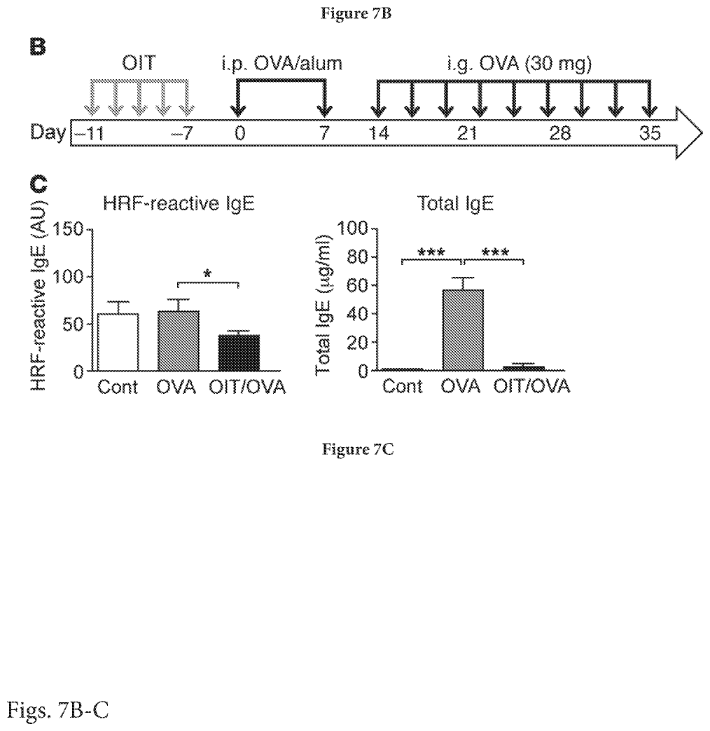

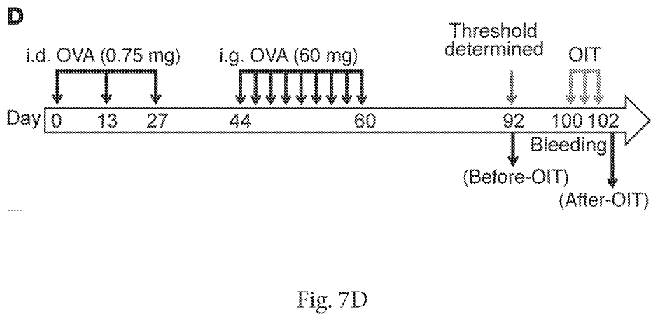

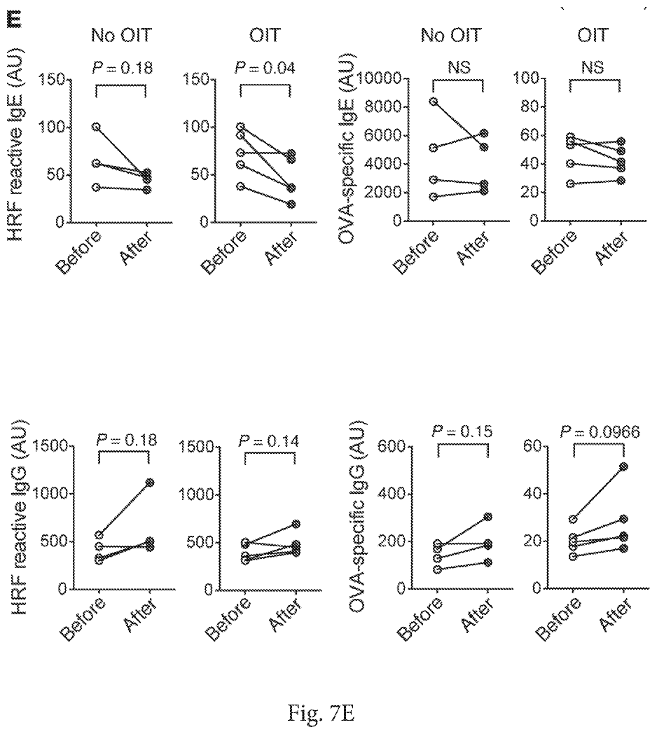

Applicant next investigated when HRF-reactive IgE levels increased during food allergy induction. Similar levels of HRF-reactive IgE were found before and after OVA challenges in non-sensitized mice and before OVA challenges in sensitized mice (FIG. 7A). Interestingly, HRF-reactive IgE levels increased by OVA challenges in OVA-sensitized mice, and HRF inhibitor (HRF-2CA) did not affect HRF-reactive IgE levels. By contrast, OVA-specific IgE levels increased before OVA challenges in OVA-sensitized mice and failed to significantly increase by OVA challenges. These results indicate that while anti-OVA IgE antibody was generated by sensitization, HRF-reactive IgE levels increased under the Th2 and inflammatory cytokines-rich environment during the elicitation phase, similar to jejunal HRF levels (FIG. 3A-FIG. 3D). By contrast, HRF-reactive IgG levels, which peaked by 9^{th} OVA challenge (FIG. 1E), were reduced after eleven OVA challenges in both non-sensitized and sensitized mice (FIG. 7A). The reduction of HRF-reactive IgG levels was not affected by HRF-2CA. However, OVA-specific IgG levels increased by sensitization and were not significantly altered by OVA challenges, similar to OVA-specific IgE levels. Thus, HRF-reactive IgG and HRF-reactive IgE levels were altered in different kinetics.

Applicant then tested the possibility provoked by the above human data that the level of HRF-reactive IgE is kept low in a desensitized state, using two murine models. In a classic tolerance model (30), five daily oral administrations of 1 mg OVA from day −11 to day −7 (OIT) prevented the OVA-induced development of allergic diarrhea in OVA-sensitized mice (FIG. 7B). By contrast, without oral pretreatment, all OVA-sensitized mice developed allergic diarrhea. HRF-reactive IgE levels were significantly reduced in tolerance-induced mice compared to non-tolerant and non-sensitized mice (FIG. 7C).

Finally, HRF-reactive IgE and HRF-reactive IgG were monitored in another desensitization model, which mimicked the human OIT more faithfully. OVA-sensitized mice were first rendered allergic by OVA challenges and then received hourly increasing amounts of OVA for 3 consecutive days (OIT) after a resting period (FIG. 7D). OIT made 80% of OVA-sensitized/OVA-challenged mice desensitized to the final gavage dose of 60 mg of OVA, whereas non-OIT treatment resulted in diarrhea reacting to 60 mg of OVA in 75% of OVA-sensitized/OVA-challenged mice. Similar to human OIT results, mouse OIT also lowered levels of HRF-reactive IgE whereas it did not change levels of OVA-specific IgE (FIG. 7E). Levels of HRF-reactive IgG and OVA-specific IgG were not changed by OIT (FIG. 7E). In non-sensitized mice, HRF-reactive IgE and HRF-reactive IgG levels were not changed by OIT and OVA-specific IgE and IgG levels were extremely low irrespective of OIT treatment (data not shown). These results, along with the human OIT data, indicate that the desensitized state induced by OIT is associated with low HRF-reactive IgE levels.

### Discussion

Little was known about the role of HRF in food allergy before the present study. This was partly because HRF knockout mice were embryonically lethal (31, 32). This study circumvented this obstacle by using HRF inhibitors that blocked HRF-IgE interactions, which clearly suppressed food allergy in mice when administered prophylactically and therapeutically.

This study does not exclude the possibility that HRF might play a role in the sensitization phase in human food allergy. Sensitization with allergen in the presence of alum skews the immune response to a Th2 type so strongly that potential involvement of HRF, which might operate in a more physiological sensitization condition, might not present itself under the employed condition. As the role of antigen-nonspecific IgE in sensitization was shown in contact sensitivity (34) and the IL-4Rα 709F model of food allergy (35) by Hans Oettgen and associates, further study is warranted on the role of HRF in the sensitization phase using a different model.

HRF dimer can exert its action via HRF-reactive IgE bound to FcεRI, leading to the activation of mast cells and basophils (19). HRF-reactive IgE levels increased during the elicitation phase, unlike antigen-specific IgE levels that increased by sensitization. Both antigen and HRF dimer can activate IgE-sensitized mast cells independently. Although the prevailing view in the field indicates that mast cell activation through FcεRI is due to receptor cross-linking with IgE and antigen (36), Applicant proposed that intestinal mast cells in food allergic mice are synergistically activated by OVA and HRF dimer (or possibly HRF oligomers). This synergistic mast cell activation by antigen and HRF could also be found in asthma, AD and other allergic diseases. Intestinal mast cells from non-sensitized mice were activated with anti-IgE, but not with OVA, indicating that a considerable proportion of the FcεRI molecules expressed on these mast cells were occupied by nonspecific IgE. By contrast, mast cells from food allergic mice were activated with OVA, indicating that a substantial proportion of the FcεRI molecules on these cells were occupied by OVA-specific IgE molecules. Since some of both OVA-specific and nonspecific IgE molecules should interact with HRF (19), activation of mast cells in food allergic mice could be triggered by both OVA and HRF dimer/multimers. Interestingly, successful OIT promoted reductions in HRF-reactive IgE without significantly affecting OVA-specific IgE levels. Therefore, reduced HRF-reactive IgE levels likely contributed to OIT-induced suppression of mast cell activation. Furthermore, this study suggests that pre-OIT levels of HRF-reactive IgE might be a useful biomarker to predict the outcome of OIT. Therefore, future study is warranted on how HRF-reactive IgE levels are regulated.

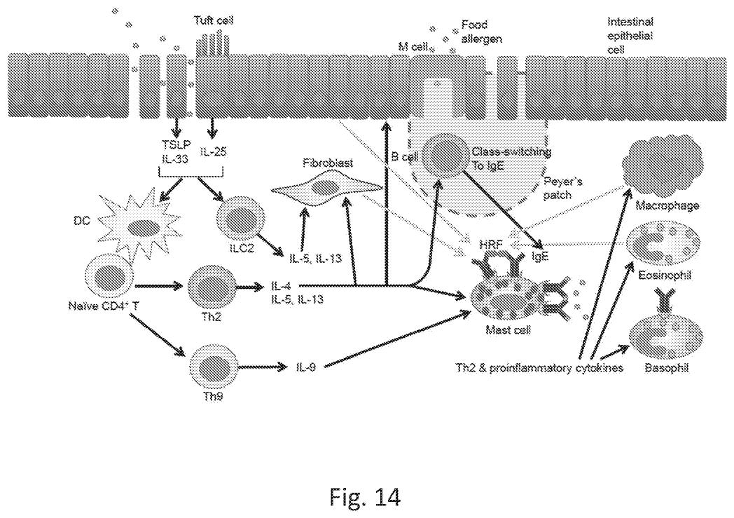

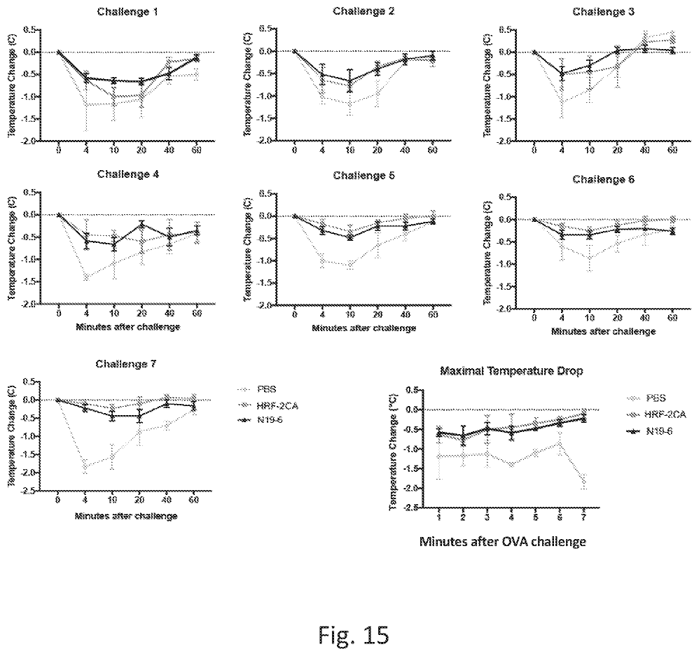

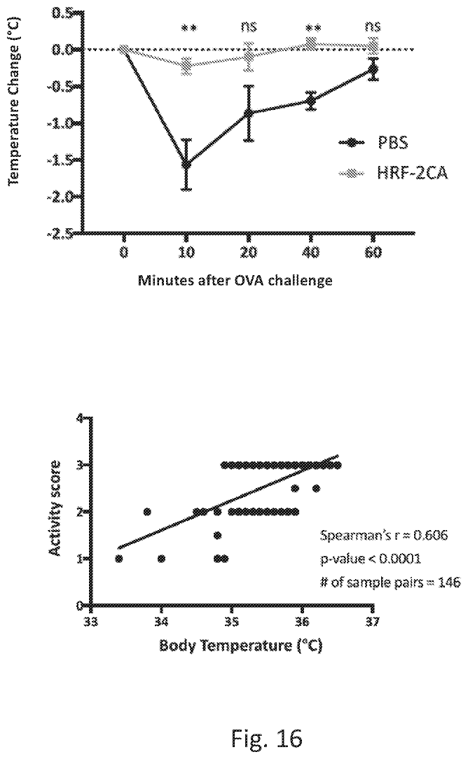

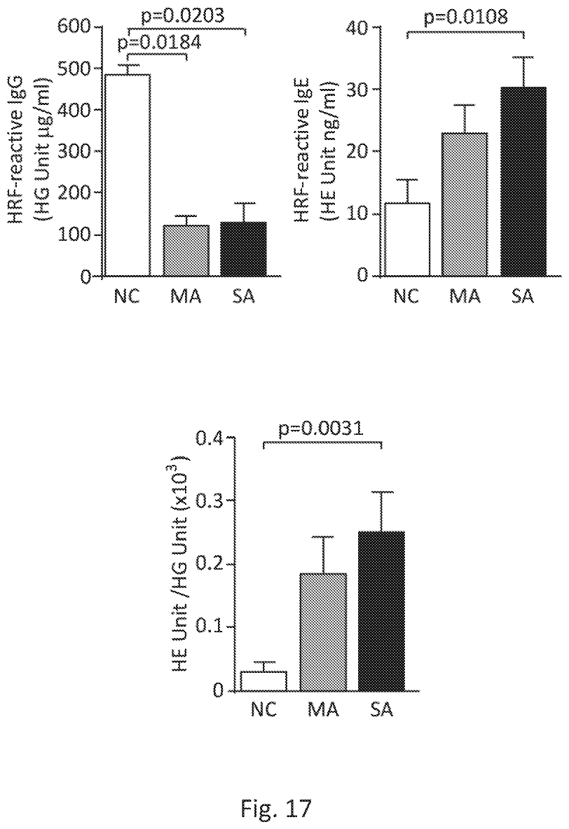

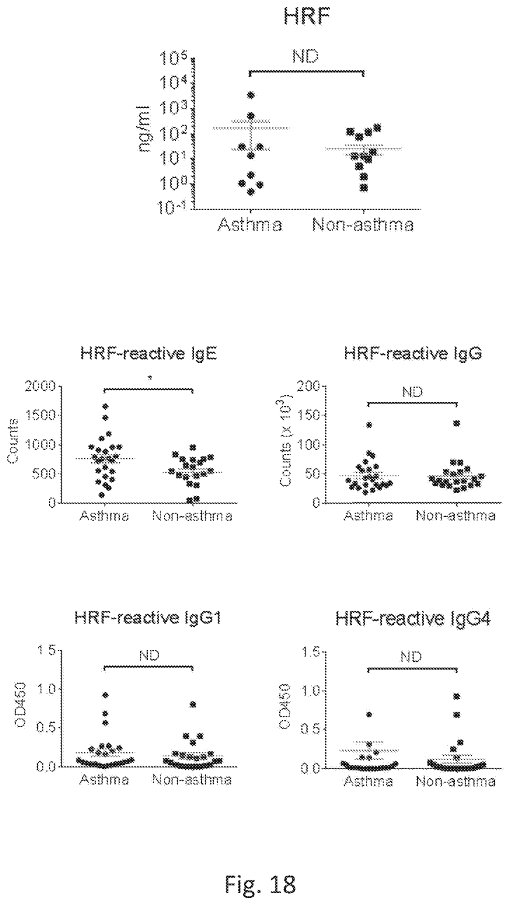

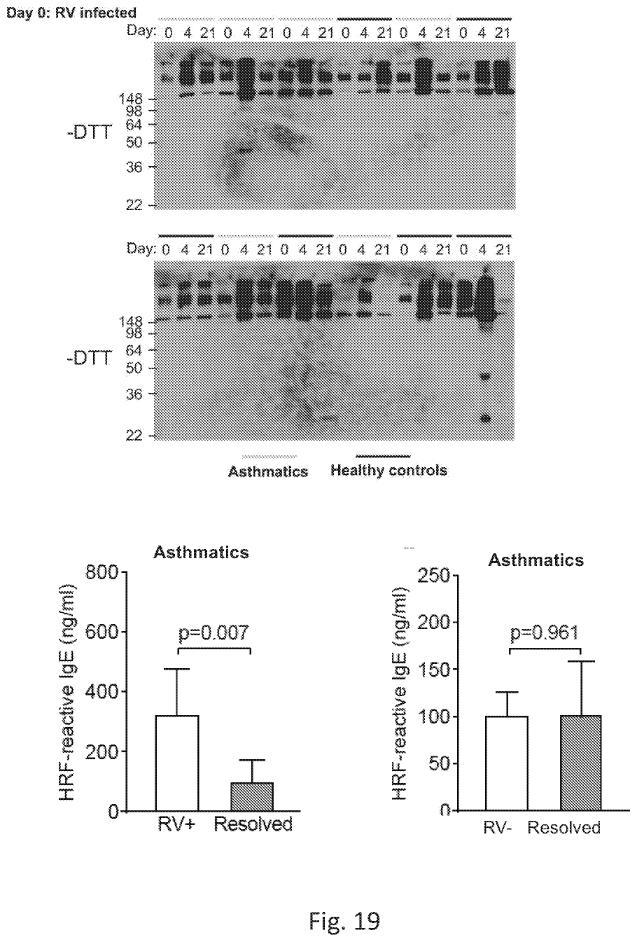

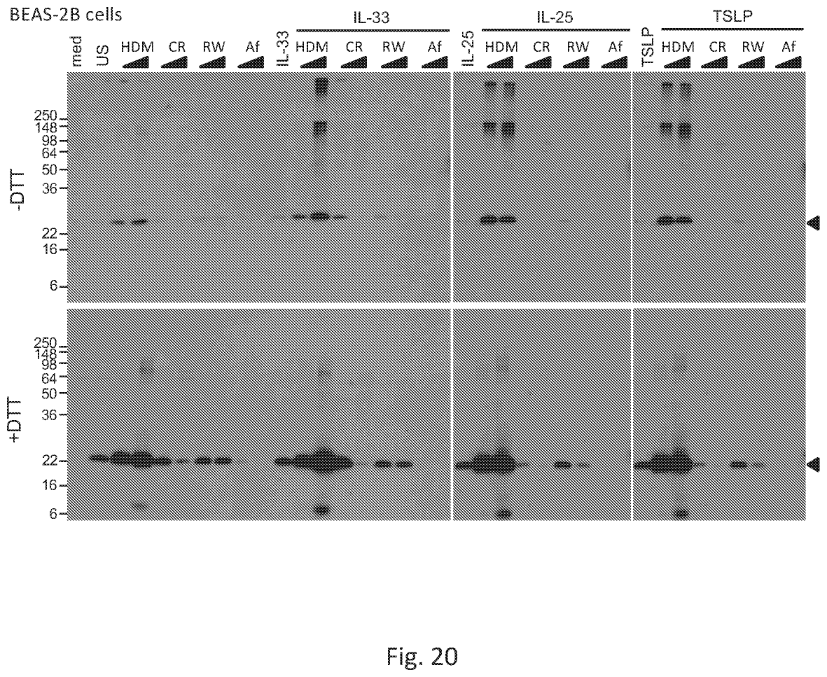

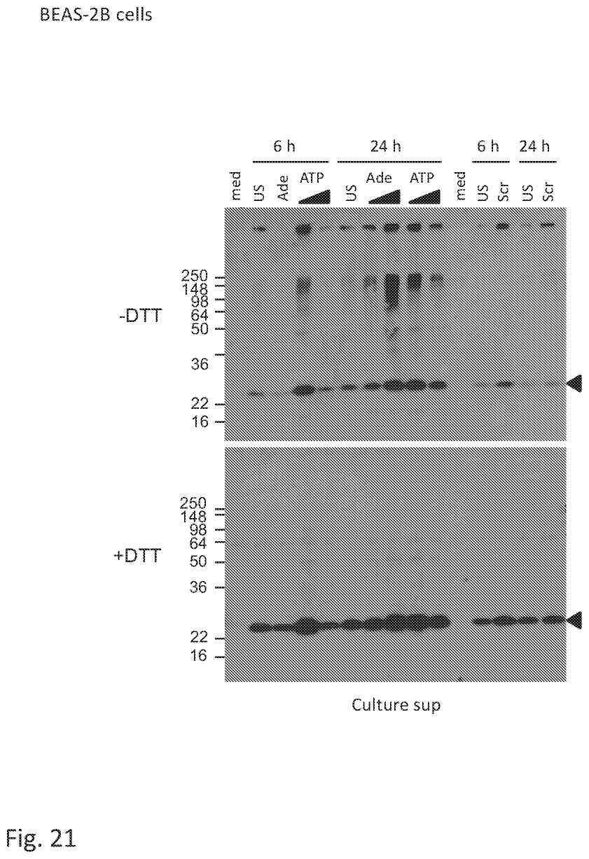

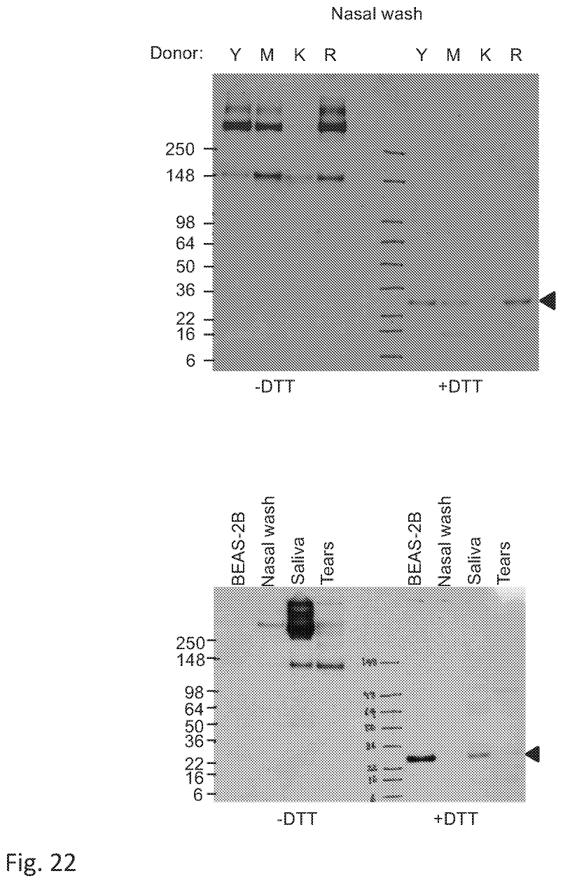

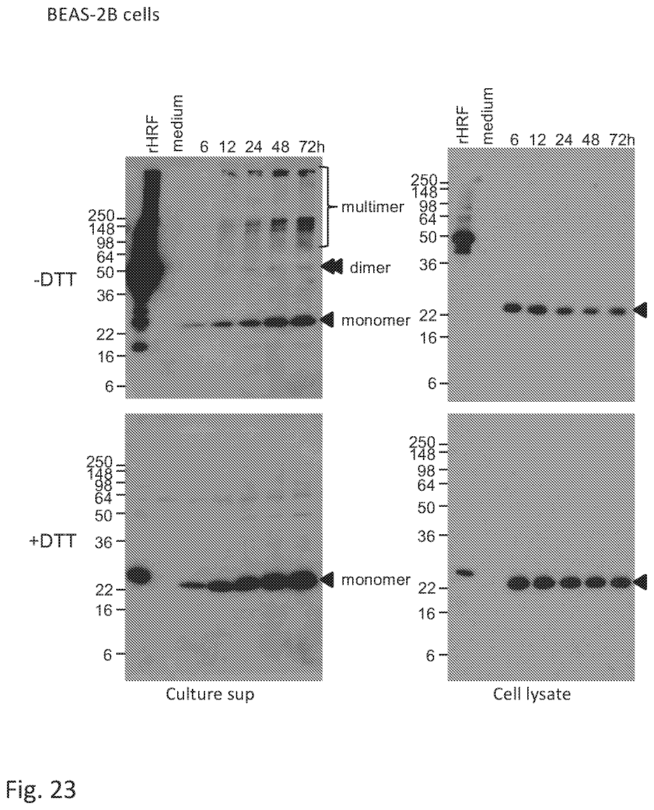

Without being bound by theory, it may be that HRF amplifies Th2 inflammation after initial inflammation is triggered by allergen-mediated activation of ILC2s and Th2 cells (FIG. 14). These cytokine-regulated highly orchestrated events lead to IgE-dependent mast cell (and basophil) activation via FcεRI.

Clinical studies are underway to investigate the efficacy of immunotherapy in food allergy. Allergen-specific sublingual, oral, and epicutaneous immunotherapies were reported to successfully treat some patients (3, 4). This reported results with egg allergy patients suggest the pathological role for HRF in human egg allergy: first, HRF-reactive IgE and HRF-reactive IgG were higher in egg allergy patients than normal controls, and OIT caused their reduction. Second, high HRF-reactive IgE levels before OIT initiation, similar to low levels of egg white-specific IgE, were predictive of OIT success. Although the good correlation of high initial HRF-reactive IgE levels with good outcomes of OIT might be counterintuitive, the disease of such patients might be highly dependent on the HRF-mediated allergic inflammation. Third, HRF-reactive IgE levels became higher only with patients who exhibited high sensitivity to egg protein after OIT. Two murine OIT models also supported this last point by showing that successful OIT keeps HRF-reactive IgE levels low. These results may encourage patients with high levels of HRF-reactive IgE and low levels of egg white-specific IgE to go through a long process of OIT with a high prospect of success. HRF-reactive IgE can serve as a biomarker for successful immunotherapy of patients with egg allergy or other food allergies.

In conclusion, this study demonstrates that HRF plays a type 2 inflammation-amplifying role during the elicitation phase of food allergy in a well-characterized murine model. HRF dimer/multimers increased in their local concentration relative to HRF monomer in the small intestine. HRF dimer could enhance IgE-mediated activation of mucosal mast cells in the small intestine of allergic mice. As further evidence for HRF/IgE-mediated mast cell activation via FcεRI, blockade of HRF-IgE interactions inhibits antigen- or HRF dimer-induced mast cell activation in vivo and ex vivo in WT, but not FcεRI-deficient, mice. Therefore, this study identifies HRF as a prophylactic and therapeutic target in food allergy and implies HRF-reactive IgE as a biomarker for the prognosis of OIT.

### Example 2

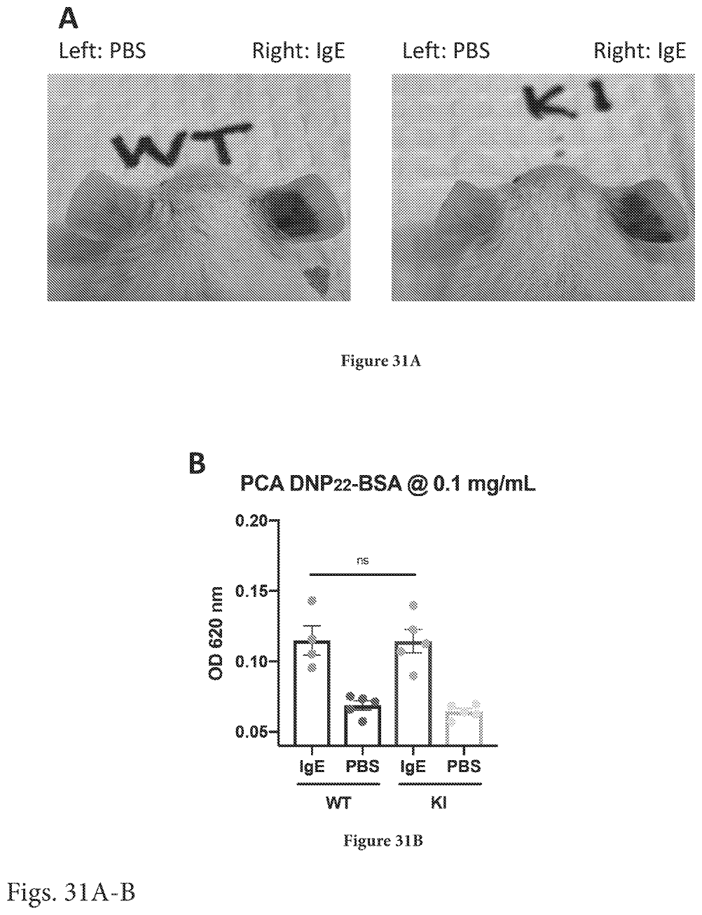

FIGS. 31A-31B illustrates passive cutaneous anaphylaxis (PCA) experiments showing significant differences between wild-type (WT) and HRF-C172A knock-in (KI) mice when stimulated with DNP₂-BSA, but not DNP₂₂-BSA. Mice were intradermally injected with anti-DNP IgE (right ear) or PBS (left ear). One day later, mice were challenged with intravenous injection of DNP₂₂-BSA (FIG. 31A) or DNP₂-BSA (FIG. 31B) (both at 0.1 mg/ml) in 1% Evans' blue dye. After 30 min, mice were sacrificed, and ears were cut and weighed, then digested overnight. Evans' blue dye was measured by spectrophotometer.

Results: Challenge with high-valency antigen (DNP₂₂-BSA) did not show significant differences in anaphylactic reactions between WT and HRF-C172A KI mice. However, anaphylactic responses induced with low-valency antigen (DNP₂-BSA) were weaker in HRF-C172A KI mice compared with WT mice. This result indicates that HRF dimer and oligomers are required for full activation of mast cells induced by IgE and low-valency antigen, but not for that by IgE and high-valency antigen.

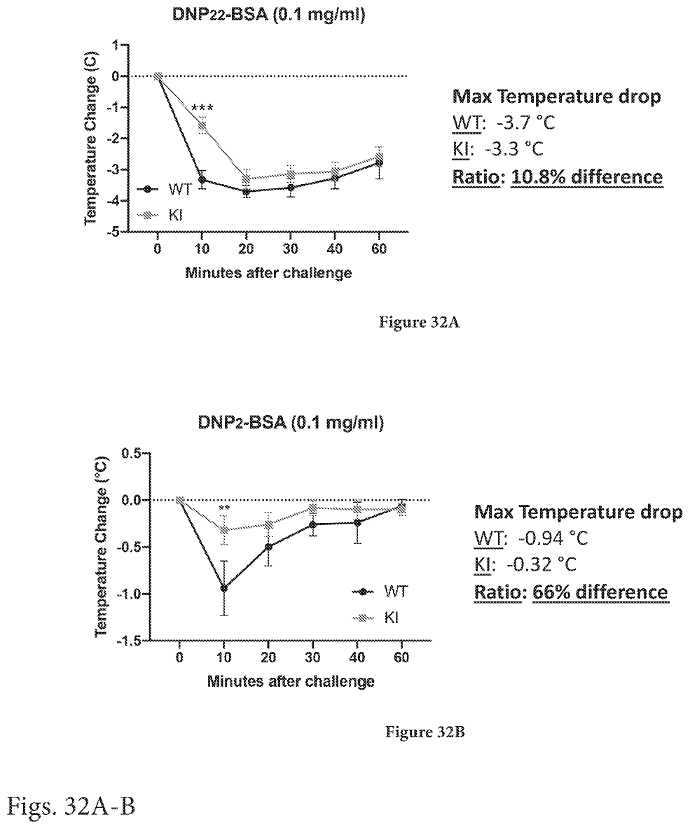

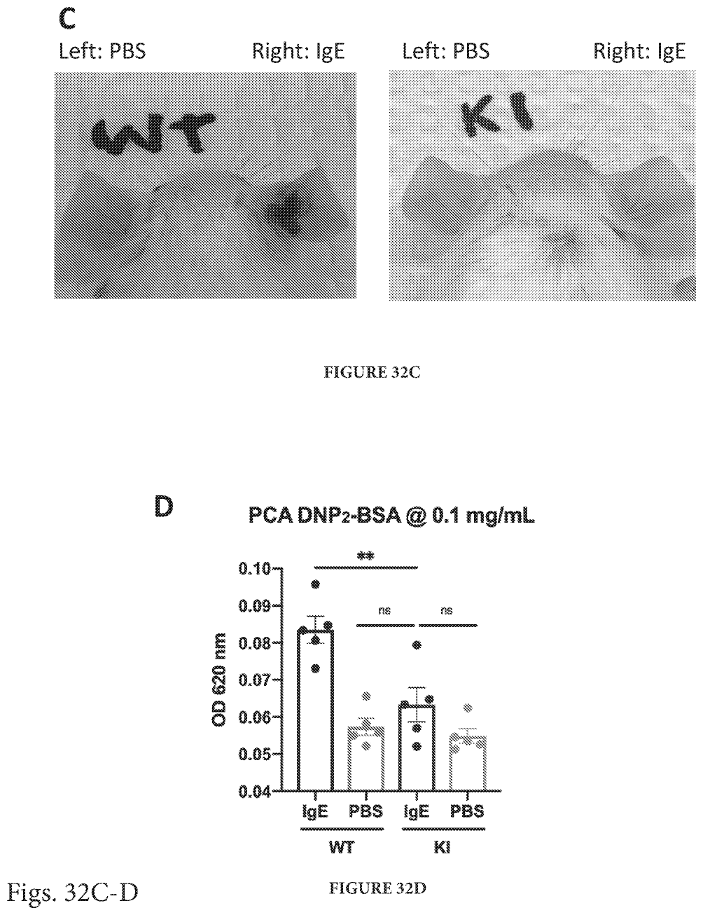

FIGS. 32A-32B shows passive systemic anaphylaxis (PSA) is less pronounced in HRF-C172A KI mice when antigen valency is low. Mice were intraperitoneally injected with anti-DNP IgE. One day later, mice were challenged with intravenous injection of DNP₂₂-BSA (FIG. 32A) or DNP₂-BSA (FIG. 32B) (0.1 mg/ml). Body temperature was monitored by an infrared thermometer for 60 min.

Results: Similar to PCA results, differences between in HRF-C172A KI and WT mice in anaphylactic responses induced with low-valency antigen (DNP₂-BSA) were larger, than when PSA was induced with high-valency antigen (DNP₂₂-BSA).

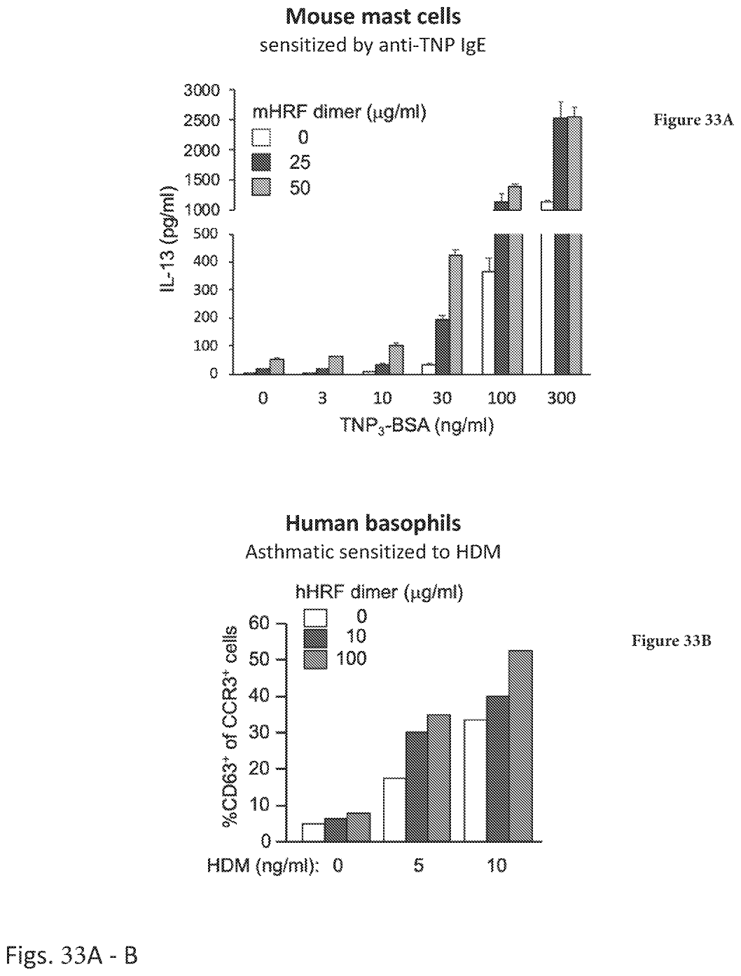

FIGS. 33A-33B show HRF dimers enhance IgE/antigen-induced activation of mast cells and basophils. FIG. 33A shows that mouse bone marrow-derived mast cells (BMMCs) were sensitized overnight with anti-TNP IgE C38-2, and then stimulated with indicated concentrations of TNP3-BSA together with mouse HRF (mHRF) dimer for 6 h. IL-13 in culture supernatants was quantified by ELISA. 50% effective concentrations (EC₅₀) of antigen were reduced by HRF dimer: 156 ng/ml for 0 μg/ml mHRF dimer; 124 ng/ml for 25 μg/ml mHRF dimer; 92 ng/ml for 50 μg/ml mHRF dimer. FIG. 33B shows heparinized blood from a house dust mite (HDM)-sensitized asthmatic was incubated with human HRF (hHRF) dimer (10 μg/ml in c) at 37° C. for 30 min. Then, HDM allergen (Dermatophagoides farinae at 10 ng/ml) was added to the cells together with FITC-anti-CD63 and APC-anti-CCR3 for 15 min. Expression of CD63 (activation marker) and CCR3 (basophil marker) was analyzed by flow cytometry. HRF monomers did not affect IgE/antigen-induced activation of mast cells or basophils (not shown).

Summary on the Role of HRF Dimer/Oligomers in Allergic Reactions:

In vitro mast cell and basophil stimulation through high-affinity IgE receptors (FcεRI) showed that HRF dimer/oligomers lower the threshold or EC₅₀ of IgE/antigen-induced activation. Consistent with these results, in vivo experiments (PCA, PSA, and ovalbumin-induced food allergy) using HRF-C172A KI mice demonstrated the role of HRF dimer/oligomers in amplifying IgE/antigen-induced allergic reactions, particularly when antigen's valency is low, which is considered to be the case in most allergic diseases.

### EQUIVALENTS

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

### REFERENCES

1. Kagan R S. Food allergy: an overview. Environ Health Perspect. 2003; 111(2):223-5.
2. Boyce J A, Assa'ad A, Burks A W, Jones S M, Sampson H A, Wood R A, et al. Guidelines for the diagnosis and management of food allergy in the United States: summary of the NIAID-sponsored expert panel report. Nutrition research. 2011; 31(1):61-75.
3. Jones S M, Burks A W, and Dupont C. State of the art on food allergen immunotherapy: oral, sublingual, and epicutaneous. The Journal of allergy and clinical immunology. 2014; 133(2):318-23.
4. Wood R A. Food allergen immunotherapy: Current status and prospects for the future. The Journal of allergy and clinical immunology. 2016; 137(4):973-82.
5. Akdis C A, and Akdis M. Mechanisms of allergen-specific immunotherapy and immune tolerance to allergens. The World Allergy Organization journal. 2015; 8(1):17.
6. Yu W, Freeland D M, and Nadeau K C. Food allergy: immune mechanisms, diagnosis and immunotherapy. Nature reviews Immunology. 2016; 16(12):751-65.
7. Helm R M. Food allergy animal models: an overview. Annals of the New York Academy of Sciences. 2002; 964:139-50.
8. Van Gramberg J L, de Veer M J, O'Hehir R E, Meeusen E N, and Bischof R J. Use of animal models to investigate major allergens associated with food allergy. Journal of allergy. 2013; 2013:635695.
9. Brandt E B, Strait R T, Hershko D, Wang Q, Muntel E E, Scribner T A, et al. Mast cells are required for experimental oral allergen-induced diarrhea. The Journal of clinical investigation. 2003; 112(11):1666-77.
10. Forbes E E, Groschwitz K, Abonia J P, Brandt E B, Cohen E, Blanchard C, et al. IL-9- and mast cell-mediated intestinal permeability predisposes to oral antigen hypersensitivity. The Journal of experimental medicine. 2008; 205(4):897-913.
11. Finkelman F D. Anaphylaxis: lessons from mouse models. The Journal of allergy and clinical immunology. 2007; 120(3):506-15; quiz 16-7.
12. Kawakami T, and Blank U. From IgE to Omalizumab. Journal of immunology. 2016; 197(11):4187-92.
13. Schneider L C, Rachid R, LeBovidge J, Blood E, Mittal M, and Umetsu D T. A pilot study of omalizumab to facilitate rapid oral desensitization in high-risk peanut-allergic patients. The Journal of allergy and clinical immunology. 2013; 132(6):1368-74.
14. Bommer U A. Cellular function and regulation of the translationally controlled tumor protein TCTP. The Open Allergy Journal. 2012; 5:19-32.
15. Kawakami T, Kashiwakura J, and Kawakami Y. Histamine-releasing factor and immunoglobulins in asthma and allergy. Allergy, Asthma & Immunology Research. 2013; 6(n1).
16. MacDonald S M, Rafnar T, Langdon J, and Lichtenstein L M. Molecular identification of an IgE-dependent histamine-releasing factor. Science. 1995; 269(5224):688-90.
17. Warner J A, Pienkowski M M, Plaut M, Norman P S, and Lichtenstein L M. Identification of histamine releasing factor(s) in the late phase of cutaneous IgE-mediated reactions. Journal of immunology. 1986; 136(7):2583-7.
18. MacDonald S M, Lichtenstein L M, Proud D, Plaut M, Naclerio R M, MacGlashan D W, et al. Studies of IgE-dependent histamine releasing factors: heterogeneity of IgE. Journal of immunology. 1987; 139(2):506-12.
19. Kashiwakura J, Ando T, Matsumoto K, Kimura M, Kitaura J, Matho M H, et al. Histamine-releasing factor has a proinflammatory role in mouse models of asthma and allergy. The Journal of clinical investigation. 2012; 122(1):218-28.
20. Chen C Y, Lee J B, Liu B, Ohta S, Wang P Y, Kartashov A V, et al. Induction of Interleukin-9-Producing Mucosal Mast Cells Promotes Susceptibility to IgE-Mediated Experimental Food Allergy. Immunity. 2015; 43(4):788-802.
21. Noti M, Kim B S, Siracusa M C, Rak G D, Kubo M, Moghaddam A E, et al. Exposure to food allergens through inflamed skin promotes intestinal food allergy through the thymic stromal lymphopoietin-basophil axis. The Journal of allergy and clinical immunology. 2014; 133(5):1390-9, 9 e1-6.
22. Daeron M, Malbec O, Latour S, Arock M, and Fridman W H. Regulation of high-affinity IgE receptor-mediated mast cell activation by murine low-affinity IgG receptors. The Journal of clinical investigation. 1995; 95(2):577-85.
23. Uermosi C, Beerli R R, Bauer M, Manolova V, Dietmeier K, Buser R B, et al. Mechanisms of allergen-specific desensitization. The Journal of allergy and clinical immunology. 2010; 126(2):375-83.
24. Tsai S H, Kinoshita M, Kusu T, Kayama H, Okumura R, Ikeda K, et al. The ectoenzyme E-NPP3 negatively regulates ATP-dependent chronic allergic responses by basophils and mast cells. Immunity. 2015; 42(2):279-93.
25. Bowler R P, and Crapo J D. Oxidative stress in allergic respiratory diseases. J Allergy Clin Immun. 2002; 110(3):349-56.
26. Kelly F J, and Sandstrom T. Air pollution, oxidative stress, and allergic response. Lancet. 2004; 363(9403):95-6.
27. Garcia N V, Umemoto E, Saito Y, Yamasaki M, Hata E, Matozaki T, et al. SIRP alpha/CD172a Regulates Eosinophil Homeostasis. Journal of immunology. 2011; 187(5):2268-77.
28. Itoh-Nagato N, Inoue Y, Nagao M, Fujisawa T, Shimojo N, Iwata T, et al. Desensitization to a whole egg by rush oral immunotherapy improves the quality of life of guardians: A multicenter, randomized, parallel-group, delayed-start design study. Allergology international: official journal of the Japanese Society of Allergology. 2017.
29. Sato S, Yanagida N, Ohtani K, Koike Y, and Ebisawa M. A review of biomarkers for predicting clinical reactivity to foods with a focus on specific immunoglobulin E antibodies. Current opinion in allergy and clinical immunology. 2015; 15(3):250-8.
30. Yamashita H, Takahashi K, Tanaka H, Nagai H, and Inagaki N. Overcoming food allergy through acquired tolerance conferred by transfer of Tregs in a murine model. Allergy. 2012; 67(2):201-9.
31. Chen S H, Wu P S, Chou C H, Yan Y T, Liu H, Weng S Y, et al. A knockout mouse approach reveals that TCTP functions as an essential factor for cell proliferation and survival in a tissue- or cell type-specific manner. Mol Biol Cell. 2007; 18(7):2525-32.
32. Susini L, Besse S, Duflaut D, Lespagnol A, Beekman C, Fiucci G, et al. TCTP protects from apoptotic cell death by antagonizing bax function. Cell death and differentiation. 2008; 15(8):1211-20.
33. Sampson H A, Broadbent K R, and Bernhisel-Broadbent J. Spontaneous release of histamine from basophils and histamine-releasing factor in patients with atopic dermatitis and food hypersensitivity. The New England journal of medicine. 1989; 321(4):228-32.
34. Bryce P J, Miller M L, Miyajima I, Tsai M, Galli S J, and Oettgen H C. Immune Sensitization in the Skin Is Enhanced by Antigen-Independent Effects of IgE. Immunity. 2004; 20(4):381-92.
35. Burton O T, Noval Rivas M, Zhou J S, Logsdon S L, Darling A R, Koleoglou K J, et al. Immunoglobulin E signal inhibition during allergen ingestion leads to reversal of established food allergy and induction of regulatory T cells. Immunity. 2014; 41(1):141-51.
36. Gould H J, Sutton B J, Beavil A J, Beavil R L, McCloskey N, Coker H A, et al. The biology of IGE and the basis of allergic disease. Annual review of immunology. 2003; 21:579-628.
37. Cayrol C, and Girard J P. IL-33: an alarmin cytokine with crucial roles in innate immunity, inflammation and allergy. Current opinion in immunology. 2014; 31:31-7.
38. Divekar R, and Kita H. Recent advances in epithelium-derived cytokines (IL-33, IL-25, and thymic stromal lymphopoietin) and allergic inflammation. Current opinion in allergy and clinical immunology. 2015; 15(1):98-103.
39. Licona-Limon P, Kim L K, Palm N W, and Flavell R A. T(H)2, allergy and group 2 innate lymphoid cells. Nature immunology. 2013; 14(6):536-42.
40. Martinez-Gonzalez I, Steer C A, and Takei F. Lung ILC2s link innate and adaptive responses in allergic inflammation. Trends in immunology. 2015; 36(3):189-95.
41. Muto T, Fukuoka A, Kabashima K, Ziegler S F, Nakanishi K, Matsushita K, et al. The role of basophils and proallergic cytokines, TSLP and IL-33, in cutaneously sensitized food allergy. International immunology. 2014; 26(10):539-49.
42. Han H, Thelen T D, Comeau M R, and Ziegler S F. Thymic stromal lymphopoietin-mediated epicutaneous inflammation promotes acute diarrhea and anaphylaxis. The Journal of clinical investigation. 2014; 124(12):5442-52.
43. Lee J B, Chen C Y, Liu B, Mugge L, Angkasekwinai P, Facchinetti V, et al. IL-25 and CD4(+) TH2 cells enhance type 2 innate lymphoid cell-derived IL-13 production, which promotes IgE-mediated experimental food allergy. The Journal of allergy and clinical immunology. 2016; 137(4):1216-25 e5.
44. Doyle A D, Jacobsen E A, Ochkur S I, Willetts L, Shim K, Neely J, et al. Homologous recombination into the eosinophil peroxidase locus generates a strain of mice expressing Cre recombinase exclusively in eosinophils. Journal of leukocyte biology. 2013; 94(1):17-24.
45. Fleischer D M, Burks A W, Vickery B P, Scurlock A M, Wood R A, Jones S M, et al. Sublingual immunotherapy for peanut allergy: a randomized, double-blind, placebo-controlled multicenter trial. J Allergy Clin Immunol 2013; 131:119-27, e1-7. 73.
46. Kim E H, Bird J A, Kulis M, Laubach S, Pons L, Shreffler W, et al. Sublingual immunotherapy for peanut allergy: clinical and immunologic evidence of desensitization. J Allergy Clin Immunol 2011; 127:640-6.e1.
47. Burks A W, Wood R A, Jones S M, Sicherer S H, Fleischer D M, Scurlock A M, et al. Sublingual immunotherapy for peanut allergy: Long-term follow-up of a randomized multicenter trial. J Allergy Clin Immunol 2015; 135:1240-8, e1-3. 74.
48. Chin S J, Vickery B P, Kulis M D, Kim E H, Varshney P, Steele P, et al. Sublingual versus oral immunotherapy for peanut-allergic children: a retrospective comparison. J Allergy Clin Immunol 2013; 132:476-8.e2
49. Dioszeghy V, Mondoulet L, Dhelft V, Ligouis M, Puteaux E, Dupont C, et al. The regulatory T cells induction by epicutaneous immunotherapy is sustained and mediates long-term protection from eosinophilic disorders in peanut-sensitized mice. Clin Exp Aller 2014; 44:867-81.
50. Mondoulet L, Dioszeghy V, Thebault C, Benhamou P H, Dupont C. Epicutaneous immunotherapy for food allergy as a novel pathway for oral tolerance induction. Immunotherapy 2015; 7:1293-305.
51. Mondoulet L, Dioszeghy V, Ligouis M, Dhelft V, Dupont C, Benhamou P H. Epicutaneous immunotherapy on intact skin using a new delivery system in a murine model of allergy. Clin Exp Allergy 2010; 40:659-67.
52. Dupont C, Kalach N, Soulaines P, Legoue-Morillon S, Piloquet H, Benhamou P H. Cow's milk epicutaneous immunotherapy in children: a pilot trial of safety, acceptability, and impact on allergic reactivity. J Allergy Clin Immunol 2010; 125:1165-7.
53. Dupont C, Bourrier T, deBlay F, Guenard-Bibault L, Sauvage C, Cousin M-O, et al. Peanut epicutaneous immunotherapy (EPIT) in peanut-allergic children: 18 months treatment in the ARACHILD Study [abstract]. J Allergy Clin Immunol 2014; 133(Suppl):AB102
54. Abramson M J, Puy R M, Weiner J M. Injection allergen immunotherapy for asthma. Cochrane Database Syst Rev. 2010; 8:CD001186.

1-36. (canceled)

37. A pharmaceutical composition comprising an agent that modulates the formation of HRF-Ig complexes to treat an allergy (optionally, a food allergy or an allergic reaction), hypersensitivity, asthma, inflammatory response or inflammation in a subject in need thereof.

38-40. (canceled)

41. A pharmaceutical composition according to claim 37, wherein the agent comprises a HRF molecule comprising a modification at amino acid residue C28, C172, or a combination thereof, wherein amino acid residues C28 and C172 correspond to positions 28 and 172 of SEQ ID NO: 4, or an equivalent thereof, wherein the equivalent comprises at least 70% sequence identity to SEQ ID NO: 4 while retaining the amino acid substitution(s) at C28 and/or C172 according to SEQ ID NO: 4.

42. A pharmaceutical composition according to claim 41, wherein the modification is a mutation, optionally from cysteine to alanine at position 28, 172, or a combination thereof.

43. (canceled)

44. (canceled)

45. A pharmaceutical composition according to claim 41, wherein the HRF molecule comprises a HRF monomer comprising at least 70% sequence identity to SEQ ID NO: 4 while still comprising the amino acid substitution(s) at C28 and/or Cl 72 according to SEQ ID NO: 4.

46. A pharmaceutical composition according to claim 45, wherein the HRF monomer comprises at least 80%, 85%, 90%, 95%, 96%, 97%, or 98% sequence identity to SEQ ID NO: 4 while retaining the amino acid substitution(s) at C28 and/or Cl 72 according to SEQ ID NO: 4.

47-61. (canceled)

62. A pharmaceutical composition according to claim 37, wherein the effective amount is:
an amount sufficient to protect against allergic reaction, hypersensitivity, asthma, inflammatory response or inflammation/or severity of allergic reaction, inflammation, food allergy, hypersensitivity, inflammatory response, decrease, reduce, inhibit, suppress, limit or control susceptibility to the food allergy, allergic reaction or hypersensitivity, or decrease, reduce, inhibit, suppress, limit or control the food allergy, allergic reaction, hypersensitivity, inflammatory response or inflammation;
an amount sufficient to decrease, reduce, inhibit, suppress, limit, control or improve the probability, severity, frequency, or duration of one or more adverse symptoms, disorders, illnesses, pathologies, diseases, or complications caused by or associated with the allergic reaction, hypersensitivity, asthma, inflammatory response or inflammation/or severity of allergic reaction, or inflammation in the subject; or
an amount that reduces or inhibits progression, severity, frequency, duration or probability of an adverse symptom of the allergic reaction, hypersensitivity, asthma, inflammatory response or inflammation/or severity of allergic reaction, or inflammation in the subject.

63. A pharmaceutical composition according to claim 62, wherein the adverse symptom comprises diarrhea, shortness of breath (dyspnea), wheezing, stridor, coughing, airway remodeling, rapid breathing (tachypnea), prolonged expiration, runny nose, rapid or increased heart rate (tachycardia), rhonchous lung, over-inflation of the chest or chest-tightness, decreased lung capacity, an acute asthmatic episode, lung, airway or respiratory mucosum inflammation, or lung, airway or respiratory mucosum tissue damage.

64. A pharmaceutical composition according to claim 37, wherein the allergic reaction is selected from: Extrinsic or intrinsic bronchial asthma; Allergic rhinitis; Onchocercal dermatitis; Atopic dermatitis; eczema; rash; allergic urticaria (e.g. hives); allergic conjunctivitis; Drug reactions; Nodules, eosinophilia, rheumatism, dermatitis, and swelling (NERDS); Eosophageal and a gastrointestinal allergy.

65. A pharmaceutical composition according to claim 62, wherein the hypersensitivity, inflammatory response or inflammation comprises a respiratory disease or disorder.

66-75. (canceled)

76. A pharmaceutical composition according to claim 37, wherein the effective amount of the therapy or the compound is from about 0.00001 mg/kg to about 10,000 mg/kg, from about 0.0001 mg/kg to about 1000 mg/kg, from about 0.001 mg/kg to about 100 mg/kg, from about 0.01 mg/kg to about 10 mg/kg, or from about 0.1 mg/kg, about 1 mg/kg body weight.

77-79. (canceled)

80. A method for the treatment of a condition from the group of: allergy (optionally, a food allergy or an allergic reaction), hypersensitivity, asthma, inflammatory response or inflammation in a subject in need thereof, comprising administering an effective amount of an agent that interferes with the formation of HRF-Ig complexes or an agent that interferes with the formation of an HRF dimer or HRF multimer to the subject, thereby treating the condition.

81-83. (canceled)

84. The method of claim 80, wherein the agent comprises a HRF molecule comprising a modification at amino acid residue C28, Cl 72, or a combination thereof, wherein amino acid residues C28 and Cl 72 correspond to positions 28 and 172 of SEQ ID NO: 4, or an equivalent thereof, wherein the equivalent comprises at least 70% sequence identity to SEQ ID NO: 4 while retaining the amino acid substitution(s) at C28 and/or Cl 72 according to SEQ ID NO: 4.

85. The method of claim 84, wherein the modification is a mutation, optionally from cysteine to alanine at position 28, 172, or a combination thereof.

86. (canceled)

87. (canceled)

88. The method of claim 84, wherein the HRF molecule comprises a HRF monomer comprising at least 70% sequence identity to SEQ ID NO: 4 while still comprising the amino acid substitution(s) at C28 and/or Cl 72 according to SEQ ID NO: 4.

89. The method of claim 88, wherein the HRF monomer comprises at least 80%, 85%, 90%, 95%, 96%, 97%, or 98% sequence identity to SEQ ID NO: 4, while still comprising the amino acid substitution(s) at C28 and/or Cl 72 according to SEQ ID NO: 4.

90. (canceled)

91. The method of claim 80, wherein the method further comprises administering an effective amount of a second drug.

92-100. (canceled)


SEQUENCE LISTING (13 sequences)

SEQ ID NO: 1 - protein, 19 aa, Unknown
  Description of Unknown HRF sequence
MIIYRDLISHDEMFSDIYK

SEQ ID NO: 2 - protein, 64 aa, Unknown
  Description of Unknown HRF sequence
QETSFTKEAYKKYIKDYMKSIKGKLEEQRPERVKPFMTGAAEQIKHILANFKNYQFFIGENMNP

SEQ ID NO: 3 - protein, 172 aa, Unknown
  Description of Unknown HRF sequence
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 4 - protein, 172 aa, Homo sapiens; identical to SEQ ID NO: 3

SEQ ID NO: 5 - protein, 172 aa, Mus musculus
MIIYRDLISHDELFSDIYKIREIADGLCLEVEGKMVSRTEGAIDDSLIGGNASAEGPEGEGTESTVVTGVDIVMNHHLQETSFTKEAYKKYIKDYMKSLKGKLEEQKPERVKPFMTGAAEQIKHILANFNNYQFFIGENMNPDGMVALLDYREDGVTPFMIFFKDGLEMEKC

SEQ ID NO: 6 - protein, 172 aa, Rattus norvegicus; identical to SEQ ID NO: 5

SEQ ID NO: 7 - protein, 172 aa, Oryctolagus cuniculus
MIIYRDLISHDEMFSDIYKIREIAGGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFYIGENMNPDGMVALLDYREDGVTPFMIFFKDGLEMEKC

SEQ ID NO: 8 - protein, 172 aa, Cavia porcellus
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGANMNPDGMVALLDYREDGVTPFMIFFKDGLEMEKC

SEQ ID NO: 9 - protein, 172 aa, Pan troglodytes; identical to SEQ ID NO: 3

SEQ ID NO: 10 - protein, 171 aa, Xenopus laevis
MIIYRDLISHDEMFSDIYKIREIADGLCLEVEGKVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 11 - protein, 172 aa, Canis lupus familiaris; identical to SEQ ID NO: 3

SEQ ID NO: 12 - protein, 172 aa, Sus scrofa; identical to SEQ ID NO: 3

SEQ ID NO: 13 - protein, 172 aa, Bos taurus
MIIYRDLISHDEMFSDIYKIREVADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC